(19) United States
(12) Patent Application Publication
(10) Pub. No.: US 2009/0023753 A1
(43) Pub. Date: Jan. 22, 2009

(54) 1,3-Thiazole-5-Carboxamides Useful as Cancer Chemotherapeutic Agents

(75) Inventors: Chih-Yuan Chuang, San Mateo, CA (US); Philip Wickens, Wallingford, CT (US); Zhengiu Hong, Milford, CT (US); Catherine Brennan, Durham, NC (US); Julie A. Dixon, Bethany, CT (US); Harold C.E. Kluender, Trumbull, CT (US); Charles Kreiman, Watertown, MA (US); Ellalahewage Kumarasinghe, Hamden, CT (US)

Correspondence Address: Bayer Health Care LLC, 400 Morgan Lane, West Haven, CT, 06516, US

(73) Assignee: Bayer HealthCare AG, Leverkusen (DE)

(21) Appl. No.: 11/885,104

(22) PCT Filed: Feb. 24, 2006
(86) PCT No.: PCT/US06/06436

§ 371 Date: Aug. 24, 2007

Related U.S. Application Data
(60) Provisional application No. 60/658,985, filed on Mar. 4, 2005.

(51) Int. Cl. A61K 31/496; A61P 35/00; C07D 417/12; A61K 31/4535; C07D 403/06

(52) U.S. Cl. 514/256; 544/333; 546/270.7; 514/342

(57) ABSTRACT

This invention relates to novel 1,3-thiazole-5-carboxamide compounds, pharmaceutical compositions containing such compounds, and the use of those compounds or compositions as cancer chemotherapeutic agents.

This invention relates to novel 1,3-thiazole-5-carboxamide compounds, pharmaceutical compositions containing such compounds, and the use of those compounds or compositions as cancer chemotherapeutic agents.

Many disease conditions are known to be associated with deregulated angiogenesis. Among these are retinopathies; chronic inflammatory disorders including arthritis; arteriosclerosis; atherosclerosis; macular degeneration; and neoplastic diseases such as cancer. In recent years, much work has been carried out to find inhibitors of angiogenesis, in hopes of developing treatments for such disorders.

WO 2004/063330 discloses (2-carboxamido)(3-amino)thiophene compounds for the treatment of cancer.

U.S. Pat. No. 6,448,277 (Novartis) discloses and claims certain benzamide derivatives for inhibition of VEGF receptor tyrosine kinase, tumor growth, and VEGF-dependent cell proliferation.

Published PCT application WO 02/066470 (Amgen) broadly discloses heterocycles containing amido and amino substituent groups, for prophylaxis and treatment of angiogenesis-mediated diseases. Published PCT application WO 2004/005279 (Amgen) discloses certain substituted anthranilic amide derivatives for the prophylaxis and treatment of angiogenesis-mediated diseases. Published PCT application WO 2004/007458 (Amgen) relates to substituted 2-alkylamine nicotinic amide derivatives and their uses in treatment of cancer and other disorders.

Published PCT application WO 00/27819 (Schering) discloses certain anthranilic acid amides for treatment of diseases that are triggered by angiogenesis. Published PCT application WO 02/090352 (Schering) relates to selective anthranilamide pyridine amides as inhibitors of VEGFR-2 and VEGFR-3. Published PCT application WO 01/81311 (Schering) relates to substituted benzoic acid amides and use thereof for the inhibition of angiogenesis.

Anthranilamides as angiogenesis inhibitors have been discussed in a series of research papers by scientists at Novartis and Schering. See Manley, et al., J. Med. Chem., 45, 5687-5693 (2002); Furet, et al., Bioorganic & Medicinal Chemistry Letters, 13, 2967-2971 (2003); Manley, et al., Cell. Mol. Biol. Lett., 8, 532-533 (2003); and Manley, et al., Biochimica et Biophysica Acta, 1697, 17-27 (2004).

EP-B-832 061 discloses benzamide derivatives and their use as vasopressin antagonists.

The present invention relates to a compound of formula (I)

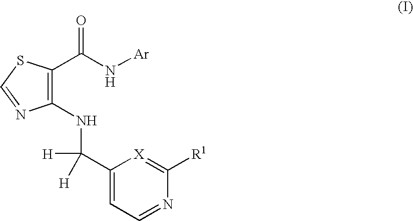

wherein
Ar is selected from the group consisting of

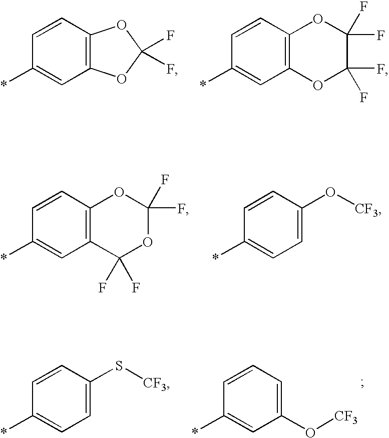

### X is CH or N;

R¹ is selected from the group consisting of

H,

halogen,

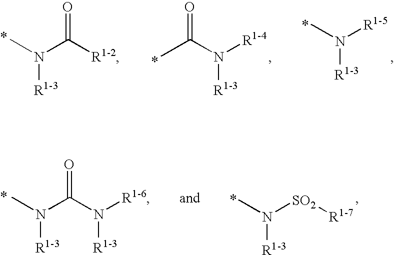

wherein
R¹⁻² is selected from the group consisting of
   H,
   (C₁-C₄)alkyl,
      wherein said (C₁-C₄)alkyl can be substituted with 0, 1, or 2 groups independently selected from
      hydroxy,
      (C₁-C₄)alkylamino,
      (C₁-C₄)acyloxy,
      (C₁-C₄)alkoxy, and
      (C₂-C₄)alkoxy substituted with 0, 1 or 2 (C₁-C₄)alkoxy groups,
   5- or 6-membered heteroaryl,
   and
   phenyl substituted with 0, 1, or 2 groups independently selected from the group consisting of (C₁-C₄)alkyl, halo, nitro, (C₁-C₄)alkoxy and cyano,
   and
   wherein said (C₁-C₄)alkyl is independently optionally substituted with F up to the perfluoro level;
R¹⁻³ is H or (C₁-C₄)alkyl;
R¹⁻⁴, R¹⁻⁵ and R¹⁻⁶ are selected from the group consisting of
   H,
   indan-5-yl,
   phenyl substituted with 0, 1, or 2 groups independently selected from the group consisting of (C₁-C₄)alkyl, halo, nitro, (C₁-C₄)alkoxy and cyano,
   5- or 6-membered heteroaryl substituted with 0, 1 or 2 groups selected from the group consisting of
      cyano,
      halo,
      nitro,
      (C₁-C₄)alkyl,
         wherein said (C₁-C₄)alkyl is optionally substituted with 0, 1, or 2 groups selected from
         (C₁-C₄)alkylamino,
         (C₁-C₄)acyloxy,
      (C₁-C₄)alkoxy,
      and
      (C₂-C₄)alkoxy substituted with up to 0, 1 or 2 (C₁-C₄)alkoxy groups,
   (C₃-C₆)cycloalkyl substituted with 0, 1 or 2 groups selected from (C₁-C₄)alkyl, (C₁-C₄)alkoxy, cyano, and halo,
   and
   (C₁-C₆)alkyl,
   wherein said (C₁-C₆)alkyl is independently substituted with 0 or 1 group selected from the group consisting of
      NH₂,
      (C₁-C₄)alkoxy,
      (C₂-C₄)alkoxy independently substituted with 0, 1, 2 or 3 (C₁-C₄)alkoxy and OH groups,
         and
         independently optionally substituted with fluorine up to the perfluoro level,
      carboxyl,
      (C₁-C₄)alkoxycarbonyl
      (C₁-C₄)alkylamino,
      aminocarbonyl,
      (C₁-C₄)alkylsulfonyl,
      phenyl substituted with 0, 1, or 2 groups independently selected from the group consisting of (C₁-C₄)alkyl, halo, nitro, (C₁-C₄)alkoxy and cyano,
      5- or 6-membered heteroaryl independently substituted with 0, 1, 2 or 3 groups selected from the group consisting of (C₁-C₄)alkyl, (C₁-C₄)alkoxy, cyano, halo, and nitro
      and
      heterocyclyl independently substituted with 0, 1, 2 or 3 groups selected from the group consisting of (C₁-C₄)alkyl, (C₁-C₄)alkoxy, cyano, and halo,
   and
   wherein said (C₁-C₆)alkyl is independently substituted with 0, 1 or 2 OH or halo groups,
   and
   wherein said (C₁-C₆)alkyl is independently optionally substituted with F up to the perfluoro level;
and
R¹⁻³ and R¹⁻⁴, R¹⁻³ and R¹⁻⁵, and R¹⁻³ and R¹⁻⁶, when attached to the same nitrogen atom, may form, together with the N atom to which they are attached, a 5- or 6-membered saturated heterocyclic ring selected from pyrrolidinyl, morpholinyl, thiomorpholinyl and piperizinyl optionally substituted on N with (C₁-C₄)alkyl,
R¹⁻⁷ is independently selected from the group consisting of
   (C₁-C₄)alkyl,
      wherein said (C₁-C₄)alkyl is substituted with 0, 1 or 2 groups selected from the group consisting of
         (C₁-C₄)alkylamino,
         (C₁-C₄)acyloxy,
         (C₁-C₄)alkoxy,
         and
         (C₂-C₄)alkoxy substituted with 0, 1 or 2 (C₁-C₄)alkoxy groups;
            or a pharmaceutically acceptable salt thereof.

The invention also relates to pharmaceutical compositions which comprise a compound of Formula (I) as defined above plus a pharmaceutically acceptable carrier.

In addition, the invention relates to a method of treating cancer comprising administering to a subject in need thereof an effective amount of a compound of Formula (I) as defined above.

Pharmaceutically acceptable salts of the compounds (I) include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, malic acid, citric acid, fumaric acid, maleic acid and benzoic acid.

Pharmaceutically acceptable salts of the compounds (I) also include salts of customary bases, such as for example and preferably alkali metal salts (for example sodium and potassium salts, alkaline earth metal salts (for example calcium and magnesium salts) and ammonium salts derived from ammonia or organic amines having 1 to 16 carbon atoms, such as illustratively and preferably ethylamine, diethylamine, triethylamine, ethyldiiso-propylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates for the purposes of the invention are those forms of the compounds that coordinate with solvent molecules to form a complex in the solid or liquid state. Hydrates are a specific form of solvates, where the coordination is with water.

For the purposes of the present invention, the substituents have the following meanings, unless otherwise specified:

The terms “(C₁-C₄)alkyl” and “(C₁-C₆)alkyl” mean a linear or branched saturated carbon group having from about 1 to about 4 C atoms or from about 1 to about 6 C atoms, respectively. Such groups include but are not limited to methyl, ethyl, n-propyl, isopropyl, n-butyl, isobutyl, sec-butyl, tert-butyl, and the like.

The term “(C₃-C₆)cycloalkyl” means a saturated carbocyclic ring group having from about 3 to about 6 C atoms. Such groups include but are not limited to cyclopropyl, cyclobutyl, cyclopentyl, cyclohexyl and the like.

The term “(C₁-C₄)alkoxy” means a linear or branched saturated carbon group having from about 1 to about 4 C atoms, said carbon group being attached to an O atom. The O atom is the point of attachment of the alkoxy substituent to the rest of the molecule. Such groups include but are not limited to methoxy, ethoxy, n-propoxy, isopropoxy, and the like.

The term “(C₁-C₄)alkylamino” means an amino group having from one or two (independently selected) (C₁-C₄)alkyl substituents, illustratively representing methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino, N-n-hexyl-N-methylamino and the like.

The term “(C₁-C₄)alkylsulfonyl” means a sulfonyl group having a (C₁-C₄)alkyl substituent, illustratively representing methylsulfonyl, ethyl sulfonyl, isopropylsulfonyl, t-butylsulfonyl, and the like.

The term “(C₁-C₄)alkoxycarbonyl” means a (C₁-C₄)alkoxygroup bound to the C atom of a carbonyl group [—C(O)—] said group being bound to the rest of the molecule, illustratively representing methoxycarbonyl, ethoxycarbonyl, n-propoxycarbonyol, i-propoxycarbonyl, t-butoxycarbonyl, and the like

The term “(C₁-C₄)acyloxy” means a (C₁-C₄)group bound to the C atom of a carboxyl group [—C(O)O—] said group being bound by the oxygen atom to the rest of the molecule illustratively representing formyloxy, acetyloxy (acetoxy), propanoyloxy, butanoyloxy, t-butanoyloxy and the like

The term “5- or 6-membered heteroaryl” means, respectively,
(1) an aromatic ring made of 5 atoms and having 1, 2, 3 or 4 heteroatom(s) each selected independently from O, N, and S, the rest being C atoms, with the proviso that there can be no more than 1 O or S atom in the heteroaryl. This heteroaryl is attached to the core molecule at any available position and is optionally substituted at any available position with the recited substituents. Such groups include pyrrole, furan, thiophene, imidazole, pyrazole, thiazole, oxazole, isoxazole, isothiazole, triazole, oxadiazole, thiadiazole, and tetrazole in all their possible isomeric forms;
or
(2) an aromatic ring made of 6 atoms, 1, 2, or 3 of which are N atoms, the rest being C, where the heterocycle is attached to the core molecule at any available C atom and is optionally substituted at any available C atom with the recited substituents. Such groups include pyridine, pyrimidine, pyridazine and triazine in all their possible isomeric forms.

The term “heterocyclyl” means a 5- or 6-membered saturated or partially saturated heterocyclic ring containing 1-2 heteroatoms selected from O, S or N, the remaining atoms being made up of C atoms, with the proviso that when there are 2 O atoms they must be nonadjacent. This heterocycle is attached to the core molecule at any available C or N atom and is optionally substituted at any available C or N atom with the recited substituents. Such groups include pyrrolidine, tetrahydrofuryl, tetrahydrothienyl, piperidinyl, tetrahydropyranyl, tetrahydrothiopyrano, piperizinyl, imidazolinyl, pyrazolinyl, morpholinyl, thiomorpholinyl and the like in all their possible isomeric forms.

The terms “halogen” and “halo” mean Cl, Br, F and I, where Cl, Br and F are preferred.

A * symbol next to a bond denotes the point of attachment in the molecule.

The compounds of this invention may contain one or more asymmetric centers, depending upon the location and nature of the various substituents desired. Asymmetric carbon atoms may be present in the (R) or (S) configuration. It is intended that all possible stereoisomers (including enantiomers and diastereomers) are included within the scope of the present invention. Preferred compounds are those with the absolute configuration of the compound of this invention which exhibits the more desirable biological activity. Separated, pure or partially purified stereoisomers or racemic mixtures of the compounds of this invention are also included within the scope of the present invention. The purification of said isomers and the separation of said stereoisomeric mixtures can be accomplished by standard techniques known in the art.

In another embodiment, the invention relates to a compound of Formula (I)

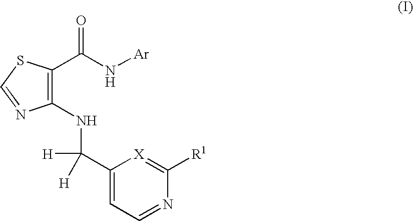

wherein
Ar is selected from the group consisting of

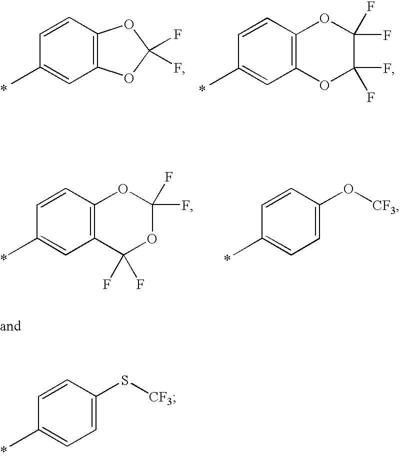

### X is CH;

R¹ is selected from the group consisting of

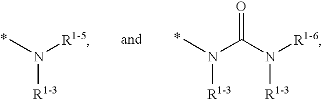

wherein
R¹⁻³ is H or (C₁-C₄)alkyl,
R¹⁻⁵ and R¹⁻⁶ are selected from the group consisting of
   H,
   indan-5-yl,
   phenyl substituted with 0, 1, or 2 groups independently selected from the group consisting of (C₁-C₄)alkyl, halo, nitro, (C₁-C₄)alkoxy and cyano,
   5- or 6-membered heteroaryl substituted with 0, 1 or 2 groups selected from the group consisting of
      cyano,
      halo,
      nitro,
      (C₁-C₄)alkyl,
         wherein said (C₁-C₄)alkyl is optionally substituted with 0, 1, or 2 groups selected from
         (C₁-C₄)alkylamino,
         (C₁-C₄)acyloxy,
      (C₁-C₄)alkoxy,
      and
      (C₂-C₄)alkoxy substituted with up to 0, 1 or 2 (C₁-C₄)alkoxy groups;
   (C₃-C₆)cycloalkyl substituted with 0, 1 or 2 groups selected from the group consisting of (C₁-C₄)alkyl, (C₁-C₄)alkoxy, cyano, and halo;
   and
   (C₁-C₆)alkyl,
   wherein said (C₁-C₆)alkyl is independently substituted with 0 or 1 group selected from the group consisting of
      NH₂,
      (C₁-C₄)alkoxy,
      (C₂-C₄)alkoxy independently substituted with 0, 1, 2 or 3 (C₁-C₄)alkoxy and OH groups,
         and
         independently optionally substituted with fluorine up to the perfluoro level,
      carboxyl,
      (C₁-C₄)alkoxycarbonyl
      (C₁-C₄)alkylamino,
      aminocarbonyl,
      (C₁-C₄)alkylsulfonyl,
      phenyl substituted with 0, 1, or 2 groups independently selected from the group consisting of (C₁-C₄)alkyl, halo, nitro, (C₁-C₄)alkoxy and cyano,
      5- or 6-membered heteroaryl independently substituted with 0, 1, 2 or 3 groups selected from the group consisting of (C₁-C₄)alkyl, (C₁-C₄)alkoxy, cyano, halo, and nitro
      and
      heterocyclyl is independently substituted with 0, 1, 2 or 3 groups selected from the group consisting of (C₁-C₄)alkyl, (C₁-C₄)alkoxy, cyano, and halo,
   and
   wherein said (C₁-C₆)alkyl is independently substituted with 0, 1 or 2 OH or halo groups,
   and
   wherein said (C₁-C₆)alkyl is independently optionally substituted with F up to the perfluoro level;
and
R¹⁻³ and R¹⁻⁵, and R¹⁻³ and R¹⁻⁶, when attached to the same nitrogen atom, may form, together with the N atom to which they are attached, a 5- or 6-membered saturated heterocyclic ring selected from pyrrolidinyl, morpholinyl, thiomorpholinyl and piperizinyl optionally substituted on N with (C₁-C₄)alkyl;
   or a pharmaceutically acceptable salt thereof.

In another embodiment, the invention relates to a compound of Formula (I)

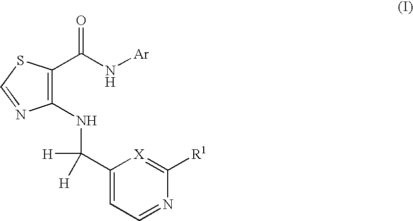

wherein

### Ar is

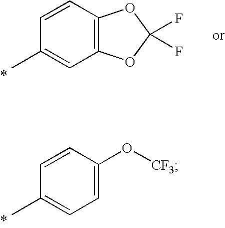

### X is CH;

R¹ is selected from

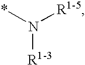

and

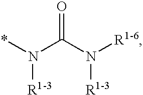

wherein
R¹⁻³ is H,
R¹⁻⁵ is (C₁-C₆)alkyl,
   wherein said (C₁-C₆)alkyl is independently substituted with 0 or 1 group selected from
      (C₁-C₄)alkoxy,
      (C₂-C₄)alkoxy independently substituted with 0, 1, or 2 (C₁-C₄)alkoxy and OH groups,
         and
         independently optionally substituted with fluorine up to the perfluoro level,
      and
      wherein said (C₁-C₆)alkyl is independently substituted with 0, 1 or 2 OH or halo groups,
      and
      wherein said (C₁-C₆)alkyl is independently optionally substituted with F up to the perfluoro level;
R¹⁻⁶ is selected from the group
   H,
   and
   (C₁-C₆)alkyl,
      wherein said (C₁-C₆)alkyl is independently substituted with 0 or 1 group selected from
         (C₁-C₄)alkoxy,
         (C₂-C₄)alkoxy independently substituted with 0, 1, 2 or 3 (C₁-C₄)alkoxy and OH groups,
         and
         independently optionally substituted with fluorine up to the perfluoro level,
   and
   wherein said (C₁-C₆)alkyl is independently substituted with 0, 1 or 2 OH or halo groups,
   and
   wherein said (C₁-C₆)alkyl is independently optionally substituted with F up to the perfluoro level;
      or a pharmaceutically acceptable salt thereof.

In another embodiment, the invention relates to a compound of Formula (I)

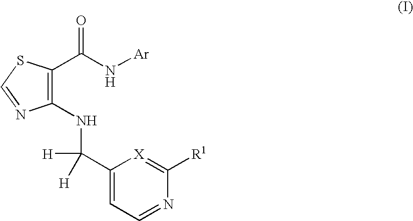

wherein

### Ar is

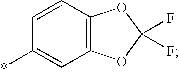

### X is CH;

R¹ is selected from

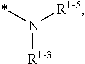

and

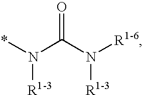

wherein
R¹⁻³ is H,
R¹⁻⁵ is methyl, ethyl, n-propyl, isopropyl, n-butyl, isobutyl, sec-butyl or tert-butyl,
   and
   wherein said methyl, ethyl, n-propyl, isopropyl, n-butyl, isobutyl, sec-butyl or tert-butyl is independently substituted with 0, 1 or 2 OH, chloro or fluoro,
   and
   wherein said methyl, ethyl, n-propyl, isopropyl, n-butyl, isobutyl, sec-butyl or tert-butyl is independently optionally substituted with F up to the perfluoro level;
R¹⁻⁶ is selected from the group
   H,
   and
   methyl, ethyl, n-propyl, isopropyl, n-butyl, isobutyl, sec-butyl or tert-butyl,
   and
   wherein said methyl, ethyl, n-propyl, isopropyl, n-butyl, isobutyl, sec-butyl or tert-butyl, is independently substituted with 0, 1 or 2 OH, chloro or fluoro,
   and
   wherein said methyl, ethyl, n-propyl, isopropyl, n-butyl, isobutyl, sec-butyl or tert-butyl, is independently optionally substituted with F up to the perfluoro level;
      or a pharmaceutically acceptable salt thereof.

### General Methods of Preparation

Compounds of Formula (I) may be prepared by synthetic procedures known to those skilled in the art or by methods analogous thereto. These methods are summarized below in Reaction Scheme 1, where

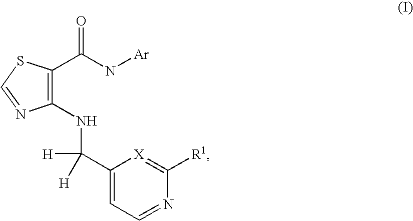

wherein Ar is
Ar is selected from

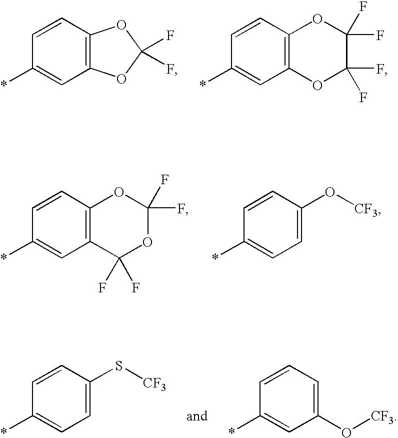

Unless otherwise specifically defined, R¹ and X have the same meanings as defined hereinabove.

The compounds of Formula (I) are generally prepared utilizing the compounds of Formula (III), (IV), (V) and (VIII) as starting materials.

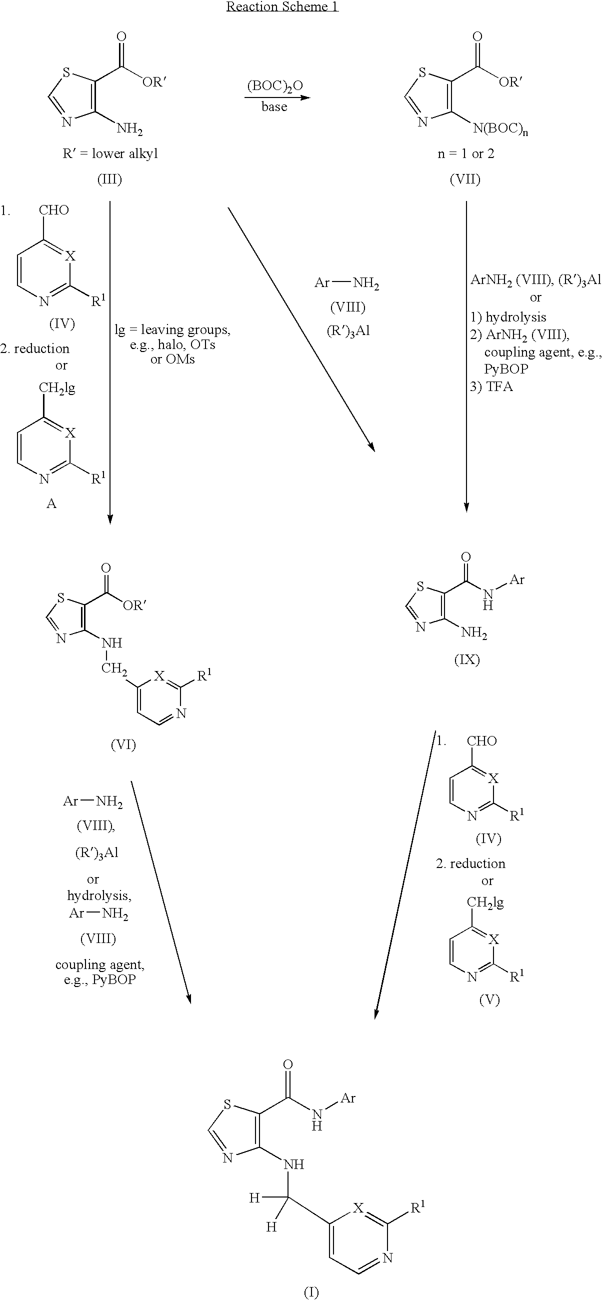

As illustrated in Reaction Scheme 1, two general synthetic routes can be used to prepare the compounds of Formula (I).

In one route, the amino group of the compound of Formula (III) is subjected to either reductive amination using a pyridine or pyrimidine carboxaldehyde of Formula (IV) and a reducing agent, such as sodium triacetoxyborohydride, or to direct N-alkylation using a pyridine or pyrimidine methyl halide, tosylate or mesylate of Formula (V) and a optional base such as pyridine or K₂CO₃, or a catalyst such as sodium iodide The product formed, Formula (VI), is then allowed to react with an aromatic amine of Formula (VIII) in the presence of a coupling agent such as (R′)₃Al (where R′=lower alkyl) giving the compound of Formula (I), or alternatively, the ester of Formula (VI) is hydrolyzed to the acid which is then coupled to the amine (VIII) using a coupling agent such as PyBOP.

In the second route, the compound of Formula (III) is converted to the aminoamide of Formula (IX) either directly by reaction with an aromatic amine of Formula (VIII) as described above, or by first protecting the amino function, e.g., as a BOC derivative (VII), and subsequent coupling with (VIII), either directly with (R′)₃Al, or via hydrolysis, and then coupling in the presence of PyBOP, followed by deprotection. The Formula (IX) compound is then converted to the Formula (I) compound using either the reductive amination method or direct N-alkylation as described above for preparation of (VI)

Starting materials of Formulae (IV), (V) and (VIII) are commercially available (e.g., Lanxess, Germany) or may be prepared by standard means well known in the art, or as described in Reaction Schemes 2-9. The preparation of starting material of Formula (III), where R″ is methyl, is described below in step 1 of Intermediate A.

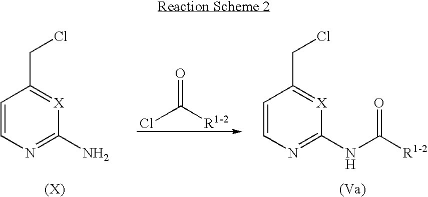

Compounds of Formula (Va),

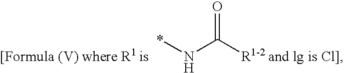

and 1 g is Cl], may be prepared as shown in Reaction Scheme 2 by reaction of an acid chloride with a chloromethyl heteroarylamine of Formula (X), generally in the presence of a base such as triethylamine.

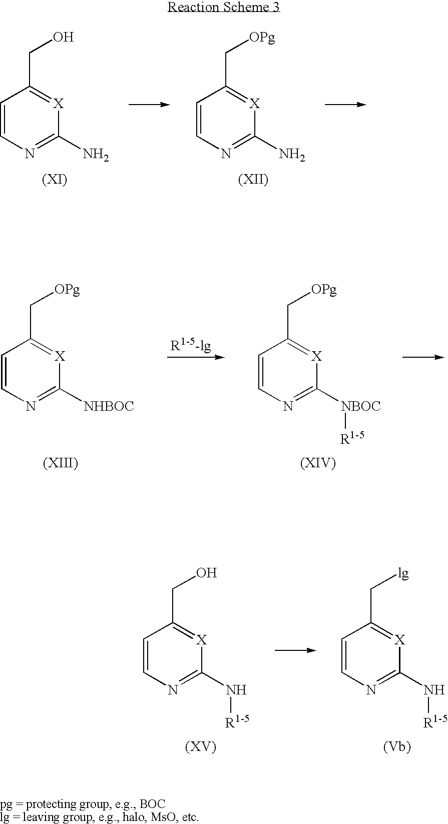

Compounds of Formula (Vb)

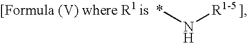

can be prepared as shown in Reaction Scheme 3 from hydroxymethylheteroaryl amines of Formula (XI). Protection of the alcohol and conversion to the BOC-derivative of Formula (XIII) is followed by N-alkylation to give the intermediate of Formula (XIV). Deprotection of the alcohol and amine, followed by conversion of the hydroxy group to a leaving group, (for example, using SOCl₂, when 1 g is Cl) gives the intermediate of Formula (Vb).

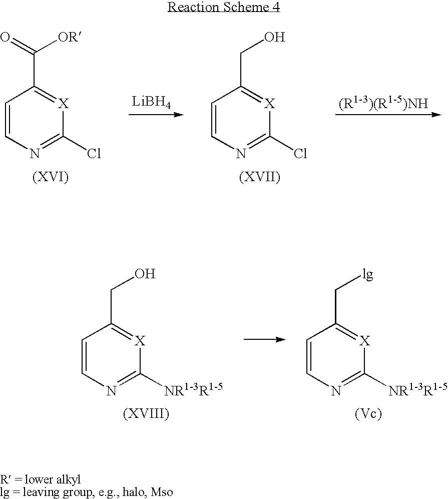

Compounds of Formula (Vc)

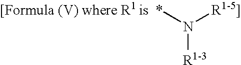

can be prepared by the route illustrated in Reaction Scheme 4. The chloroheteroarylcarboxylic acid derivative of Formula (XVI) is reduced to the chloroheteroaryl alcohol of Formula (XVII) with a standard reagent such as lithium borohydride. Reaction of the chloro compound with an amine of Formula (R¹⁻³)(R¹⁻⁵)NH gives the intermediate alcohol of Formula (XVIII). Conversion of this alcohol to a leaving group, e.g. mesylate, completes the synthesis of the compound of Formula (Vc)

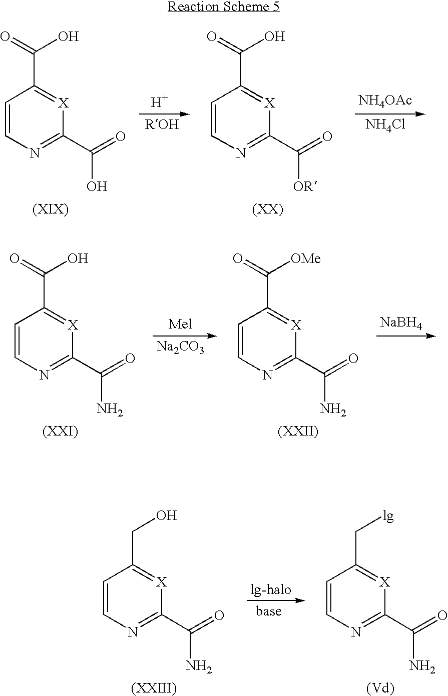

Compounds of Formula (Vd)

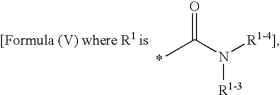

can be prepared as shown in Reaction Scheme 5 from the dicarboxylic acid of Formula (XIX) by conversion through the half acid ester (XX) to the acid amide of Formula (XXI). Esterification of (XXI) provides (XXII) which can be reduced with sodium borohydride to the alcohol (XXIII) and then converted to the Formula (Vd) compound, using for example MsCl and a base such as triethylamine.

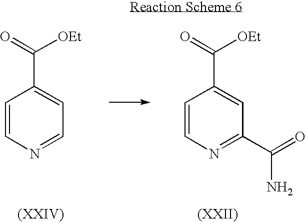

An alternate method of preparing the pyridine amide ester of Formula (XXII) is via the Minisci reaction shown in Reaction Scheme 6 in which the pyridine carboxylic acid ester is stirred in formamide with cooling to 10° C. in the presence of an equivalent of concentrated H₂SO₄, FeSO₄ and H₂O₂.

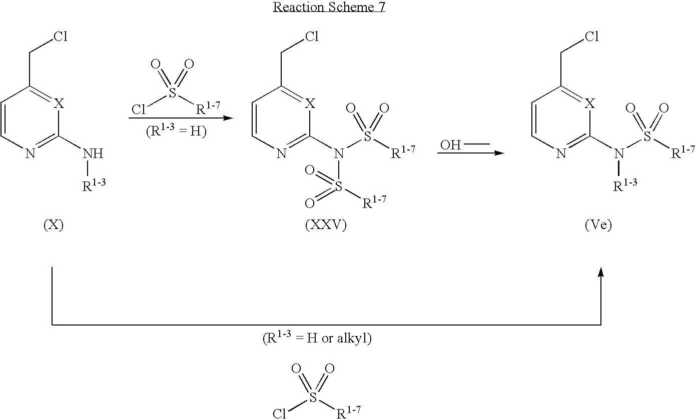

Compounds of Formula (Ve)

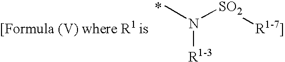

can be prepared by the route shown in Reaction Scheme 7. Starting from the intermediate of Formula (X), the sulfonamide (Ve) may be prepared in a manner analogous to that described for Formula (Va), by reaction of (X) with a sulfonyl chloride in the presence of a base. The bis-sulfonylated compound (XXV), if formed, may be converted to (Ve) if necessary, by reaction with aqueous base.

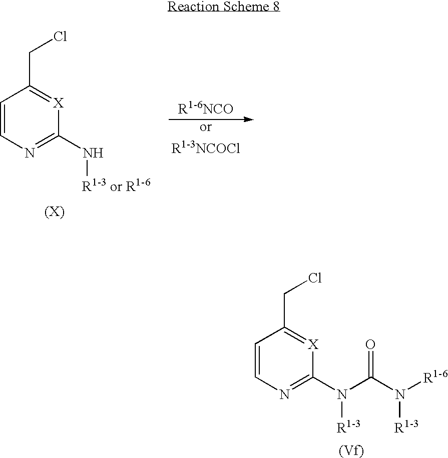

Compounds of Formula (Vf)

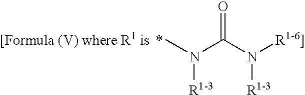

can be prepared by the route shown in Reaction Scheme 8. In the case that the R¹⁻³ on the right is H, the intermediate of Formula (X) is allowed to react with an isocyanate of Formula R¹⁻⁶NCO in an aprotic solvent such as dichloromethane. In the case that the R¹⁻³ on the right is alkyl, or that R¹⁻³ and R¹⁻⁶ are combined in a cyclic structure, the intermediate of Formula (X) is allowed to react with a carbamoyl chloride Formula R¹⁻⁶ R¹⁻³NCOCl in an aprotic solvent such as dichloromethane in the presence of a base such as triethylamine or potassium carbonate. The use of a starting material of Formula (X) in which the R¹⁻³ on the left is alkyl results in the preparation of a urea of structure Vf where R¹ is

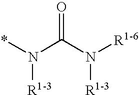

in which the R¹⁻³ group on the left is alkyl. In the case that both R¹⁻³ on the right and R¹⁻⁶ are H, benzoyl isocyanate is reacted with the intermediate of Formula (X) to give a protected urea of Formula (Vf). The benzoyl group is removed from the final molecule after combining Vf with the core molecule. In the cases that the isocyanate of Formula R¹⁻⁶NCO is not commercially available (and R¹⁻³ is H), it can conveniently be prepared by treatment of the amine of Formula R¹⁻⁶NH₂, wherein R¹⁻⁶ is aryl or heteroaryl, with phosgene, diphosgene or triphosgene in a suitable solvent such as ethyl acetate. When R¹⁻⁶ is alkyl or substituted alkyl, the preferred method is to treat the corresponding alkyl halide or dialkyl sulfate with inorganic cyanates. These methods, as well as others, are well known to those skilled in the art and examples are described in S. R. Sandler and W. Karo “Organic Functional Group Preparations,” vol 12, 2^{nd} ed., p 364-375, 1983, Academic Press and references cited therein.

In the cases that the carbamoyl chloride of Formula R¹⁻⁶ R¹⁻³NCOCl is not commercially available, it can conveniently be prepared by treatment of the amine of Formula R¹⁻⁶ R¹⁻³ NH with phosgene, diphosgene or triphosgene in a suitable solvent such as dichloromethane at 0-4° C. Optionally, the N-benzyl protected amine of Formula R¹⁻⁶ R¹⁻³NCH₂(C₅H₆) can be reacted with triphosgene as described by M. G. Banwell, et al, J. Org. Chem. 2003, 68, 613-616.

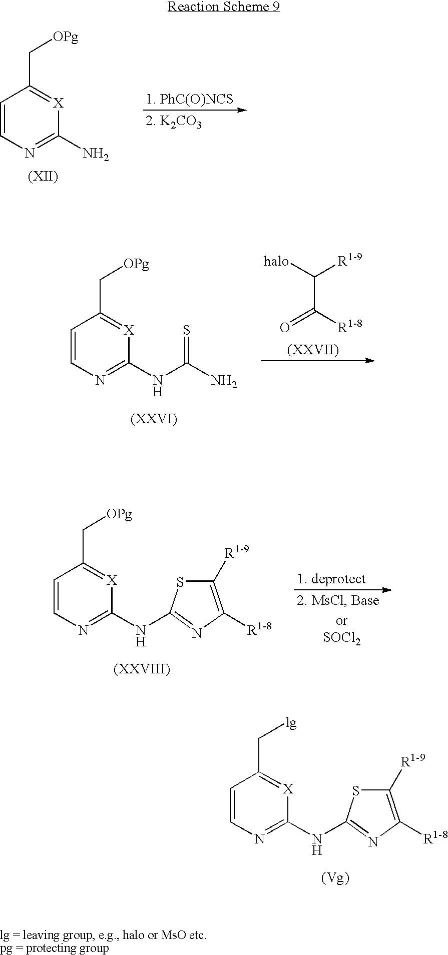

Compounds of Formula (Vg)

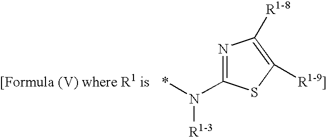

can be prepared by the route shown in Reaction Scheme 9. The intermediate of Formula (XII), prepared as in Reaction Scheme 3, is allowed to react with benzoyl isothiocyanate, followed by a base such as potassium carbonate, to form the thiourea intermediate of Formula (XXVI). This thiourea (XXVI) is then allowed to react with a 2-haloketone of Formula (XXVII) in the presence of a base, to form the thiazole intermediate of Formula (XXVIII). Deprotection and conversion of the alcohol to a leaving group, e.g. mesylate, completes the synthesis of the intermediate of Formula (Vg).

A variety of compounds of Formula (I) can be prepared by elaboration of compounds, also of Formula (I), prepared by the above schemes. These elaboration methods are illustrated below in Reaction Schemes 10-13.

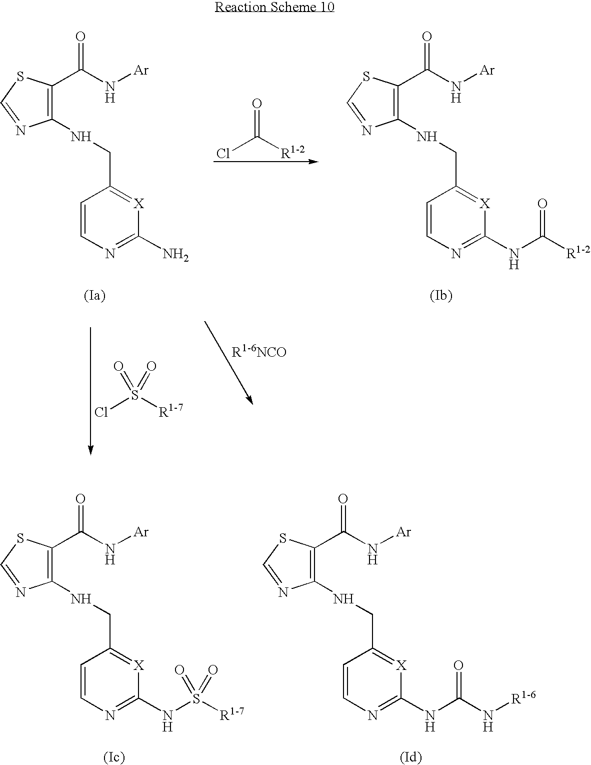

For example, the amino compound of Formula (Ia) can be converted to the amide compound of Formula (Ib), the sulfonamide of Formula (Ic) or the urea of Formula (Id) as shown in Reaction Scheme 10, by reaction with an acid chloride, sulfonyl chloride or isocyanate, respectively.

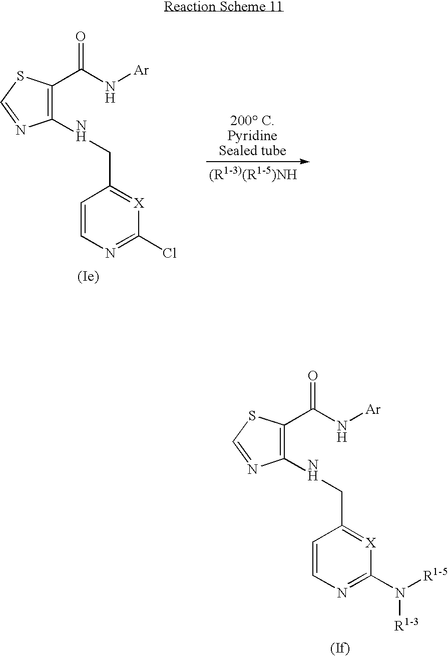

Additionally, the chloro compound of Formula (Ie) can be converted to the substituted amino compound of Formula (If) by reaction with an amine and a base such as pyridine in a sealed tube at elevated temperatures.

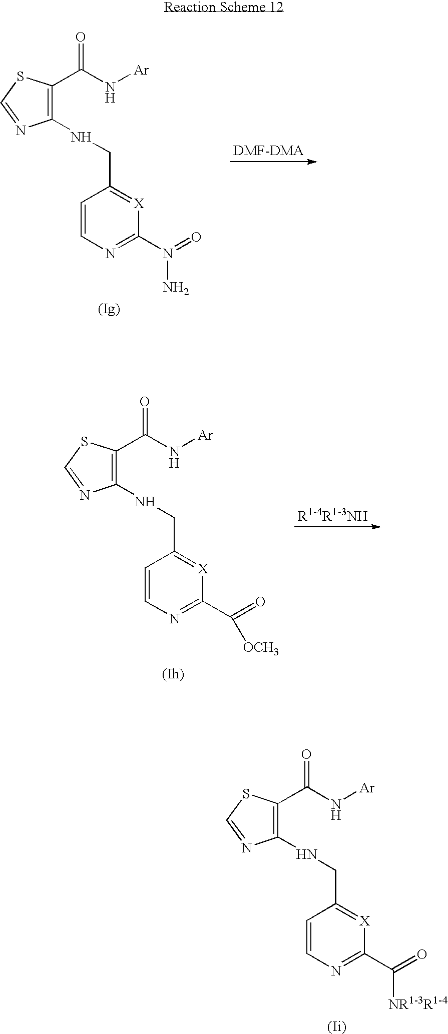

Esters of Formula (Ie) and substituted amides of Formula (II) may be prepared from the unsubstituted amide of Formula (Ig) by the sequence illustrated in Reaction Scheme 12. Reaction of the amide (Ig) with dimethylformamide-dimethylacetal in methanol provides the ester of Formula (Ih); reaction of the ester with a substituted amine gives the amide of Formula (II).

Generally, a desired salt of a compound of this invention can be prepared in situ during the final isolation and purification of a compound by means well known in the art. Or, a desired salt can be prepared by separately reacting the purified compound in its free base form with a suitable organic or inorganic acid and isolating the salt thus formed. These methods are conventional and would be readily apparent to one skilled in the art.

Additionally, sensitive or reactive groups on the compound of this invention may need to be protected and deprotected during any of the above methods. Protecting groups in general may be added and removed by conventional methods well known in the art (see, for example, T. W. Greene and P. G. M. Wuts, Protective Groups in Organic Synthesis; Wiley: New York, (1999).

By using the above illustrated general schemes and choosing the appropriate starting materials the compounds of the invention may be prepared. To further illustrate the invention, the following specific examples are provided, but are not meant to limit the scope of the invention in any way.

### A. EXAMPLES

### Abbreviations and Acronyms

When the following abbreviations are used throughout the disclosure, they have the following meaning:
bm broad multiplet
BOC t-butoxycarbonyl
bp boiling point
bs broad singlet
bt broad triplet
CD₃CN acetonitrile-d₃
CD₃OD methanol-d₄
Celite® registered trademark of Celite Corp. brand of diatomaceous earth
d doublet
DMSO-d₆ dimethylsulfoxide-d₆
DMF N,N-dimethylformamide
EtOAc ethyl acetate
h hour(s)
¹H NMR proton nuclear magnetic resonance
HPLC high performance liquid chromatography
LCMS liquid chromatography/mass spectroscopy
min minute(s)
mL milliliter(s)
Ms methanesulfonyl
m/z mass to charge ratio
PyBOP benzotriazole-1-yl-oxy-tris-pyrrolidino-phosphonium hexafluorophosphate
rt room temperature
RT retention time (HPLC or LCMS)
singlet
t triplet
TFA trifluoroacetic acid
THF tetrahydrofuran
TLC thin layer chromatography
Ts p-toluenesulfonyl

### General Analytical Procedures

The structure of representative compounds of this invention were confirmed using the following procedures.

Electron impact mass spectra (EI-MS) were obtained with a Hewlett Packard 5989A mass spectrometer equipped with a Hewlett Packard 5890 Gas Chromatograph with a J & W DB-5 column (0.25 μM coating; 30 m×0.25 mm). The ion source is maintained at 250° C. and spectra were scanned from 50-800 amu at 2 sec per scan.

High pressure liquid chromatography-electrospray mass spectra (LC-MS) were obtained using either a:

(A) Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 min at a flowrate of 1.0 mL/min is used with an initial hold of 0.5 min and a final hold at 95% B of 0.5 min. Total run time is 6.5 min.
or
(B) Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data is also acquired as an analog channel. The eluents were A: 2% acetonitrile in water with 0.02% TFA and B: 2% water in acetonitrile with 0.018% TFA. Gradient elution from 10% B to 90% over 3.5 min at a flowrate of 1.5 mL/min is used with an initial hold of 0.5 min and a final hold at 90% B of 0.5 min. Total run time is 4.8 min. An extra switching valve is used for column switching and regeneration.

Routine one-dimensional NMR spectroscopy is performed on 400 MHz Varian Mercury-plus spectrometers. The samples were dissolved in deuterated solvents obtained from Cambridge Isotope Labs, and transferred to 5 mm ID Wilmad NMR tubes. The spectra were acquired at 293 K. The chemical shifts were recorded on the ppm scale and were referenced to the appropriate solvent signals, such as 2.49 ppm for DMSO-d₆, 1.93 ppm for CD₃CN, 3.30 ppm for CD₃OD 5.32 ppm for CD₂Cl₂ and 7.26 ppm for CDCl₃ for ¹H spectra.

### Preparation of Intermediates

### Intermediate A

### Preparation of 4-amino-N-(2,2-difluoro-1,3-benzodioxol-5-yl)-1,3-thiazole-5-carboxamide

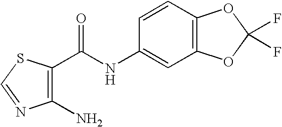

### Step 1: Preparation of methyl 4-amino-1,3-thiazole-5-carboxylate

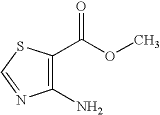

This material was prepared using the general procedure of Baldwin, John J.; Engelhardt, Edward L.; Hirschmann, Ralph; Ponticello, Gerald S.; Atkinson, Joseph G.; Wasson, Burton K.; Sweet, Charles S.; Scriabine, Alexander. Heterocyclic analogs of the antihypertensive β-adrenergic blocking agent (S)-2-[3-(tert-butylamino)-2-hydroxypropoxy]-3-cyanopyridine. Journal of Medicinal Chemistry (1980), 23(1), 65-70, which is hereby incorporated by reference, as follows: A mixture of methyl 4-amino-2-(methylthio)-1,3-thiazole-5-carboxylate (2.55 g, 12.5 mmol) in methanol (50 mL) was stirred with warming until all material was dissolved and then allowed to cool to ambient temperature before zinc dust (4.90 g, 75 mmol) was added. A 3 N hydrochloric acid solution in methanol was prepared by cautiously adding the appropriate quantity of concentrated aqueous HCl to methanol. The reaction flask with a rapidly stirred suspension of zinc and starting material in methanol was continuously flushed with nitrogen as 2.5 mL portions of the HCl solution were added at 10-minute intervals. During this addition, rapid evolution of gas ensued which was passed from the reaction flask into a bubbler of bleach to capture evolved methanethiol. Periodic HPLC analysis indicated that 1.5 h after the last of ten portions of HCl was added, most of the starting material was gone. The reaction mixture was slowly poured into a rapidly stirred suspension of Celite® in 200 mL saturated aqueous sodium carbonate. The resulting mixture was filtered and the solids were rinsed with minimal methanol. Water (100 mL) was added to the filtrate that was then extracted three times with dichloromethane. Combined extracts were washed with saturated brine, dried (Na₂SO₄) and evaporated in vacuo to yield technical grade title compound (684 mg, 34%) which appeared reasonably pure by NMR and HPLC analysis and was used directly in the next reaction.

¹H NMR (300 MHz, DMSO-d₆) δ 8.93 (s, 1H), 7.00 (bs, 2H) and 3.72 ppm (s, 3H); HPLC RT (min) 1.43.

### Step 2: Preparation of methyl 4-[bis(tert-butoxycarbonyl)amino]-1,3-thiazole-5-carboxylate

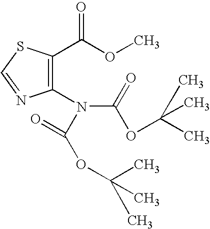

A slurry of methyl 4-amino-1,3-thiazole-5-carboxylate (2.83 g, 17.9 mmol, technical grade) in dichloromethane (65 mL) was stirred under nitrogen in a 500 mL flask as di-tert-butyl dicarbonate (8.40 g, 38.5 mmol) was added, followed by dichloromethane (5 mL), followed by N,N-dimethylpyridin-4-amine (218 mg, 1.8 mmol), at which point all materials quickly dissolved to give a yellow solution. After stirring 27.5 h the solution was loaded directly onto a 120 g silica gel column which had been equilibrated with hexane. Products were eluted with a gradient from 0-40% ethyl acetate in hexane at 70 mL/min. Fractions containing the title compound, which eluted at 22-40%, were evaporated to yield 3.48 g (54%) of pure material as a white solid and 0.53 g (8%) mixed fractions. In separate experiments when purified methyl 4-amino-1,3-thiazole-5-carboxylate was used, the yield was as high as 88%.

¹H NMR (300 MHz, DMSO-d₆) δ 9.25 (s, 1H), 3.80 (s, 3H) and 1.35 ppm (s, 18H); HPLC RT (min) 3.13.

### Step 3: Preparation of 4-[(tert-butoxycarbonyl)amino]-1,3-thiazole-5-carboxylic acid

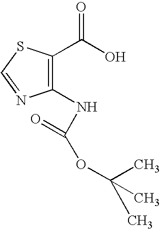

A solution of methyl 4-[bis(tert-butoxycarbonyl)amino]-1,3-thiazole-5-carboxylate (3.88 g, 10.8 mmol) in ethanol (100 mL) plus tetrahydrofuran (50 mL) was stirred under nitrogen as aqueous 1 N sodium hydroxide (100 mL) was added. The resulting mixture was stirred at 70° C. for 3 h, cooled to ambient temperature and then evaporated in vacuo. The residue was mixed with 1M aqueous monobasic potassium phosphate (400 mL) and stirred rapidly as the pH was adjusted to 5.0 by slow addition of 2 N aqueous HCl. The product was extracted with six 300 mL portions of dichloromethane then the aqueous phase was saturated by addition of solid sodium chloride and extracted three more times with ethyl acetate. The combined extracts were dried (Na₂SO₄) and evaporated in vacuo, and then toluene was added to the residue which was evaporated again to yield 2.54 g (96%) of pure dry product ready for the next step.

¹HNMR (300 MHz, DMSO-d₆) δ 9.3 (s, 1H), 9.1 (s, 1H) and 1.47 ppm (s, 9H); HPLC RT (min) 2.02.

### Step 4: Preparation of tert-butyl (5-{[(2,2-difluoro-1,3-benzodioxol-5-yl)amino]carbonyl}-1,3-thiazol-4-yl)carbamate

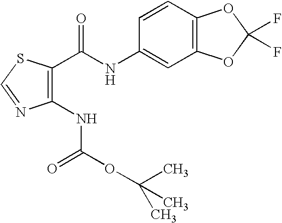

A solution of 4-[(tert-butoxycarbonyl)amino]-1,3-thiazole-5-carboxylic acid (200 mg, 0.82 mmol), 2,2-difluoro-1,3-benzodioxol-5-amine (170 mg, 0.98 mmol), triethylamine (0.34 mL, 2.46 mmol) and (1H-1,2,3-benzotriazol-1-yloxy)(tripyrrolidin-1-yl)phosphonium hexafluorophosphate (PyBOP, 511 mg, 0.98 mmol) in anhydrous dimethylformamide (2 mL) was stirred at 60° C. for 22 h under nitrogen and then cooled. The resulting solution was diluted with ethyl acetate and washed with water and then saturated brine. The organic layer was dried (Na₂SO₄) and evaporated in vacuo. The resulting residue was chromatographed on 60 mL of silica gel using dichloromethane to charge the crude product to the top of the column and a gradient from 30-100% ethyl acetate in hexane was used to elute the products. Fractions containing the title compound were combined and evaporated to yield 254 mg of product, determined to be about 95% pure by NMR and HPLC. This was used directly in the next step.

¹H NMR (300 MHz, DMSO-d₆) δ 10.28 (s, 1H), 9.87 (s, 1H), 9.08 (s, 1H), 7.73 (d, 1H, meta coupling, 7.3-7.36 (dd, 2H) and 1.38 ppm (s, 9H); HPLC RT (min) 3.51.

### Step 5: Preparation of 4-amino-N-(2,2-difluoro-1,3-benzodioxol-5-yl)-1,3-thiazole-5-carboxamide

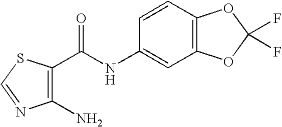

A solution of tert-butyl (5-{[(2,2-difluoro-1,3-benzodioxol-5-yl)amino]carbonyl}-1,3-thiazol-4-yl)carbamate (242 mg, 0.61 mmol) in dichloromethane (20 mL) plus trifluoroacetic acid (2 mL) was stirred under nitrogen for 1.75 h and evaporated in vacuo. The residue was dissolved in dichloromethane and washed with saturated aqueous NaHCO₃. The aqueous phase was back extracted with dichloromethane and the combined extract was dried (Na₂SO₄) and evaporated in vacuo to yield 150 mg (83%) of the title compound as an off white solid which appeared to be about 90% pure by NMR and LCMS.

¹H NMR (300 MHz, DMSO-d₆) δ 9.65 (s, 1H), 8.88 (s, 1H), 7.73 (d, 1H, meta coupling), 7.3-7.34 (dd, 2H) and 7.06 ppm (bs, 2H); ES-MS m/z 300.0 [M+H]⁺, HPLC RT (min) 2.91.

### Intermediate B

### Preparation of 4-amino-N-(2,2,3,3-tetrafluoro-2,3-dihydro-1,4-benzodioxin-6-yl)-1,3-thiazole-5-carboxamide

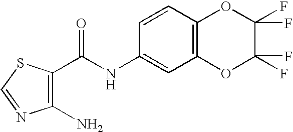

This intermediate was prepared by using the method described above for the preparation of Intermediate A but using 2,2,3,3-tetrafluoro-2,3-dihydro-1,4-benzodioxin-6-amine rather than 2,2-difluoro-1,3-benzodioxol-5-amine in Step 4. The pure product was characterized by NMR spectroscopy.

¹H NMR (300 MHz, DMSO-d₆) δ 9.76 (s, 1H), 8.94 (s, 1H), 7.85 (d, 1H, meta coupling), 7.54 (dd, 1H, meta and ortho coupling), 7.42 (d, 1H, ortho coupling) and 7.14 ppm (bs, 2H).

### Intermediate B-2

### Preparation of 4-amino-N-(2,2,4,4-tetrafluoro-4H-1,3-benzodioxin-6-yl)-1,3-thiazole-5-carboxamide

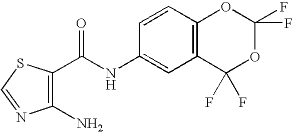

This intermediate was prepared by using the method described above for the preparation of Intermediate A but using 2,2,4,4-tetrafluoro-4H-1,3-benzodioxin-6-amine rather than 2,2-difluoro-1,3-benzodioxol-5-amine in Step 4.

ES-MS m/z 350.1 [M+H]⁺, HPLC RT (min) 3.17.

### Intermediate B-3

### Preparation of 4-amino-N-[4-(trifluoromethoxy)phenyl]-1,3-thiazole-5-carboxamide

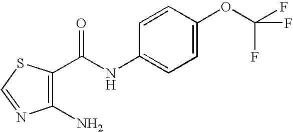

This intermediate was prepared by using the method described above for the preparation of Intermediate A but using 4-(trifluoromethoxy)aniline rather than 2,2-difluoro-1,3-benzodioxol-5-amine in Step 4.

¹H NMR (300 MHz, DMSO-d₆) δ 9.66 (s, 1H), 8.92 (s, 1H), 7.74 (d, 2H), 7.30 (d, 2H), and 7.10 ppm (bs, 2H).

### Intermediate B-4

### Preparation of 4-amino-N-[3-(trifluoromethoxy)phenyl]-1,3-thiazole-5-carboxamide

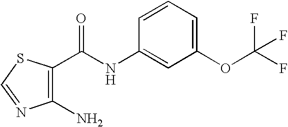

This intermediate was prepared by using the method described above for the preparation of Intermediate A but using 3-(trifluoromethoxy)aniline rather than 2,2-difluoro-1,3-benzodioxol-5-amine in Step 4.

¹H NMR (300 MHz, DMSO-d₆) δ 9.74 (s, 1H), 8.94 (s, 1H), 7.80 (s, 1H), 7.66 (d, 1H), 7.40 (t, 1H), 7.14 (bs, 2H) and 7.01 ppm (d, 1H).

Intermediate B-5

### Preparation of 4-amino-N-[4-[(trifluoromethyl)thio]phenyl]-1,3-thiazole-5-carboxamide

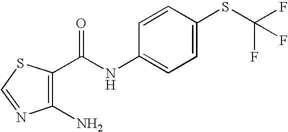

This intermediate was prepared by using the method described above for the preparation of Intermediate A but using 4-[(trifluoromethyl)thio]aniline rather than 2,2-difluoro-1,3-benzodioxol-5-amine in Step 4.

¹H NMR (300 MHz, DMSO-d₆) δ 9.78 (s, 1H), 8.94 (s, 1H), 7.82 (d, 2H), 7.65 (d, 2H), and 7.15 ppm (bs, 2H); ES-MS m/z 320.1 [M+H]⁺, HPLC RT (min) 3.19.

### Intermediate C

### Preparation of [2-(aminocarbonyl)pyridin-4-yl]methyl methanesulfonate

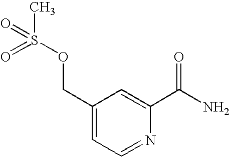

### Step 1: Preparation of ethyl 2-(aminocarbonyl)isonicotinate

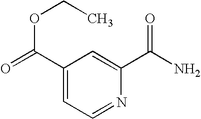

A solution of ethyl isonicotinate (25.2 mL, 165 mmol) in formamide (200 mL) was stirred with ice/methanol bath cooling as concentrated sulfuric acid (8.80 mL, 165 mmol) was added. Ferrous sulfate heptahydrate (69 g, 248 mmol) and hydrogen peroxide (25.6 mL of 30% in water) were added slowly over 25 min in alternating portions such that the temperature of the mixture was kept between 8-10.5° C. During this addition small pieces of dry ice were added to the bath to keep the reaction temperature in the desired range. After addition was complete, the ice bath was removed and the dark mixture was stirred for 2 h without cooling and then poured into a solution of trisodium citrate dihydrate (80.6 g) in water (700 mL) and then residues left in the reaction flask were washed out with a little methanol and water. The resulting mixture was rapidly stirred in a large flask as solid NaHCO₃ was added slowly, portion-wise, until the mixture was basic. Some saturated aqueous NaHCO₃ was added to make the mixture more basic and then it was vacuum filtered through Celite® and the solids were washed down with three 200 mL portions of dichloromethane. The phases of the filtrate were separated and the aqueous layer was extracted twice with dichloromethane. The combined extract was dried (Na₂SO₄) and evaporated in vacuo. The resulting solid residue was washed with ether/hexane (200 mL, 1:30) twice with warming and sonication followed by cooling and filtration to yield 13.9 g (44%) of pure title compound. The wash solutions, which contained some highly contaminated desired product, were discarded.

¹H NMR (300 MHz, DMSO-d₆) δ 8.83 (d, 1H), 8.39 (d, 1H, meta coupling), 8.24 (bs, 1H), 8.00 (d, 1H), 7.81 (bs, 1H), 4.39 (q, 2H) and 1.37 ppm (t, 3H); ES-MS m/z 195.0 [M+H]⁺, HPLC RT (min) 1.83.

### Step 2: Preparation of 4-(hydroxymethyl)pyridine-2-carboxamide

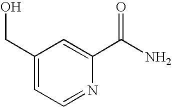

A slurry of ethyl 2-(aminocarbonyl)isonicotinate (5.00 g, 25.8 mmol) in absolute ethanol (150 mL) was stirred under nitrogen as sodium borohydride (2.92 g, 77.2 mmol) was added. After 22 h stirring at ambient temperature, the reaction was carefully quenched by addition of 17 mL of saturated aqueous ammonium chloride followed by stirring until the bubbling stopped and then evaporation in vacuo to leave a white solid residue. Saturated aqueous sodium chloride (80 mL) was added followed by five extractions with 200 mL portions of ethyl acetate. Combined extracts were dried (Na₂SO₄) and evaporated in vacuo to yield 3.85 g (98%) of pure title compound as a white solid.

¹H NMR (300 MHz, DMSO-d₆) δ 8.52 (d, 1H), 8.00 (s, 1H), 8.07 (bs, 1H), 7.46 (d, 1H), 7.60 (bs, 1H), 5.54 (t, 1H) and 4.60 ppm (d, 2H); ES-MS m/z 154.0 [M+H, weak signal]⁺, HPLC RT (min) 1.05.

### Step 3: Preparation of [2-(aminocarbonyl)pyridin-4-yl]methyl methanesulfonate

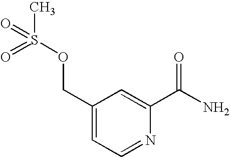

4-(hydroxymethyl)pyridine-2-carboxamide (1.00 g, 6.57 mmol) was dissolved in ethyl acetate (80 mL) and then cooled to 0° C. with stirring under nitrogen in an ice bath before triethylamine (1.37 mL, 9.86 mmol) was added, followed by methanesulfonyl chloride (0.66 mL, 8.54 mmol, added dropwise over 7 min). The ice bath was removed and the resulting suspension was stirred 2 h, and then the reaction mixture was poured into 60 mL water and stirred rapidly for 10 min. The phases were separated and the aqueous was extracted twice more with ethyl acetate. Each extract was washed with brine and the combined extracts were dried (Na₂SO₄) and evaporated in vacuo to yield 1.50 g (99%) of pure product as a fine white solid which turned pink on storage. Re-assay by NMR after such color change did not show significant decomposition.

¹H NMR (300 MHz, DMSO-d₆) δ 8.64 (d, 1H), 8.06 (s, 1H), 8.14 (bs, 1H), 7.6 (d, 1H), 7.70 (bs, 1H), 5.41 (s, 2H) and 3.33 ppm (s, overlaps with water in solvent).

### Intermediate D

### Preparation of {2-[(methylamino)carbonyl]pyridin-4-yl}methyl methanesulfonate

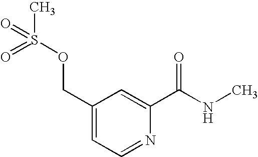

This compound is made in the same manor as Intermediate C but starting with methyl formamide rather than formamide in step 1 and methanesulfonic anhydride rather than by methanesulfonyl chloride in step 3.

¹H NMR (300 MHz, DMSO-d₆) δ 8.80 (bs, 1H), 8.64 (d, 1H), 8.03 (s, 1H), 7.58 (d, 1H), 5.41 (s, 2H), 3.29 (s, 3H) and 2.80 ppm (d, 3H); ES-MS m/z 145.1 [M+H]⁺, HPLC RT (min) 1.43.

### Intermediate E

### Preparation of 2-{[4-(chloromethyl)pyridin-2-yl]amino}-2-oxoethyl acetate

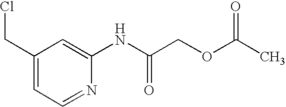

### Step 1: Preparation of 4-(chloromethyl)pyridin-2-amine

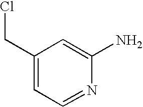

(2-Aminopyridin-4-yl)methanol (11.2 g, 90 mmol) was stirred in a flask with ice bath cooling as thionyl chloride (65.8 mL, 902 mmol) was slowly added. After about 10 mL was added, the temperature increased suddenly to about 50° C. and addition was halted as the mixture was broken up so that stirring could continue as the rest of the thionyl chloride was added. The cooling bath was then removed and the reaction was stirred for 2 h at ambient temperature before it was evaporated in vacuo and then toluene was added twice and evaporated each time in vacuo to yield the hydrochloride salt of the title compound. A suspension of this material in dichloromethane (150 mL) was stirred with saturated aqueous sodium bicarbonate (150 mL) for 1.5 h. The phases were separated and the organic extract was washed twice with water, once with brine and then dried (Na₂SO₄) and evaporated in vacuo to yield 10.71 g (83%) of pure title compound.

¹H NMR (300 MHz, DMSO-d₆) δ 7.87 (d, 1H), 6.48 (d, 1H), 6.45 (s, 1H), 6.04 (s, 2H) and 4.60 ppm (s, 2H); ES-MS m/z 143.2 [M+H]⁺, HPLC RT (min) 1.34.

### Step 2: Preparation of 2-{[4-(chloromethyl)pyridin-2-yl]amino}-2-oxoethyl acetate

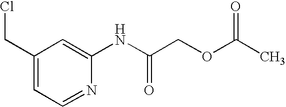

A suspension of 4-(chloromethyl)pyridin-2-amine (2.50 g, 10 mmol) and triethylamine (11.7 mL) in dichloroethane (10 mL) was stirred under nitrogen with ice bath cooling as acetoxyacetyl chloride (1.86 mL, 17 mmol) was added slowly over 10 min. After 2 h stirring with cooling, TLC showed no starting material but three major product spots. The mixture was diluted with dichloromethane and washed with water and then brine. It was dried (Na₂SO₄) and evaporated in vacuo. The residue was purified by chromatography on silica gel using a gradient from 0-3% methanol in dichloromethane to yield 0.62 g (18%) of the correct and pure title compound.

¹H NMR (300 MHz, DMSO-d₆) δ 10.75 (s, 1H), 8.30 (d, 1H), 8.10 (bs, 1H), 7.17 (d, 1H), 4.79 (s, 2H), 4.71 (s, 2H) and 2.13 ppm (s, 3H); ES-MS m/z 243.1 [M+H]⁺, HPLC RT (min) 1.87.

### Intermediate F

### Preparation of N-[4-(chloromethyl)pyridin-2-yl]acetamide

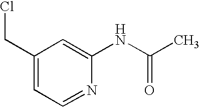

By using the methods described for preparation of Intermediate E and by substituting acetyl chloride instead of acetoxyacetyl chloride in step 2, Intermediate F was prepared from 2.30 g of 4-(chloromethyl)pyridin-2-amine and proportional amounts of other reagents. The yield of title compound was 2.0 g (67%) after silica gel chromatography. Even though examination of this material by NMR spectroscopy indicated that it was a mixture of the desired compound and the diacylated product N-acetyl-N-[4-(chloromethyl)pyridin-2-yl]acetamide (about 45:55), it was used as is in the next reaction and side products were separated by chromatography after the subsequent reaction.

¹H NMR (300 MHz, CD₂Cl₂) δ 8.33 (bs, 1H), 7.41 (d, 1H), 7.30 (s, 1H), 7.10 (d, 1H), 4.65 (s, 2H) and 2.20 ppm (s, 3H); ES-MS m/z 185.0 [M+H]⁺, HPLC RT (min) 1.16. Signals for the contaminating diacyl compound show at ¹H NMR (300 MHz, CD₂Cl₂) δ 8.56 (d, 1H), 8.18 (s, 1H), 78.24 (d, 1H), 4.75 (s, 2H) and 2.25 ppm (s, 6H); ES-MS m/z no significant M+H⁺ ion, HPLC RT (min) 0.97. Because of the closeness of the % content of the two compounds, it is possible that some of the NMR peak assignments have been switched between the desired material and the contaminant.

### Intermediate G

### Preparation of N-[4-(chloromethyl)pyridin-2-yl]-2-methoxyacetamide

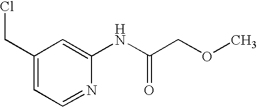

By using the methods described for preparation of Intermediate E and by substituting 2-methoxyacetyl chloride instead of acetoxyacetyl chloride in step 2, Intermediate G was prepared from 731 mg of 4-(chloromethyl)pyridin-2-amine and proportional amounts of other reagents. The yield of pure title compound was 397 mg (45%) after silica gel chromatography using a gradient from 0-40% ethyl acetate in hexane.

¹H NMR (300 MHz, CDCl₃) δ 9.00 (bs, 1H), 8.31 (d, 1H), 8.30 (s, 1H), 7.13 (d, 1H), 4.55 (s, 2H), 4.06 (s, 2H) and 3.51 ppm (s, 3H); ES-MS m/z 215.0 [M+H]⁺, HPLC RT (min) 0.71.

### Intermediate H

### Preparation of N-[4-(chloromethyl)pyridin-2-yl]-2-(2-methoxyethoxy)acetamide

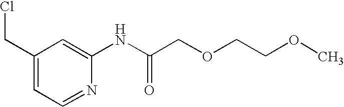

By using the methods described for preparation of Intermediate E and by substituting 2-(2-methoxyethoxy)acetyl chloride instead of acetoxyacetyl chloride in step 2, Intermediate H was prepared from 599 mg of 4-(chloromethyl)pyridin-2-amine and proportional amounts of other reagents. The yield of pure title compound was 314 mg (29%) after silica gel chromatography twice, first using a gradient from 2-3% methanol in dichloromethane, and then a second chromatography of the best fractions using a gradient from 0-40% ethyl acetate in hexane.

¹H NMR (300 MHz, CD₂Cl₂) δ 9.39 (bs, 1H), 8.30 (d, 1H), 8.29 (s, 1H), 7.13 (d, 1H), 4.59 (s, 2H), 4.14 (s, 2H), 3.76 (t, 2H), 3.60 (t, 2H) and 3.44 ppm (s, 3H); ES-MS m/z 259.1 [M+H]⁺, HPLC RT (min) 1.46.

### Intermediate I

### Preparation of N-[4-(chloromethyl)pyridin-2-yl]-2-methoxypropanamide

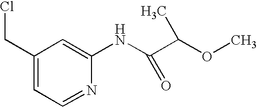

### Step 1: Preparation of 2-methoxypropanoic acid

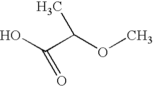

Sodium methoxide in methanol (25%, 16 mL) was added to a stirred solution of 2-bromopropionic acid (19.6 mmol) in methanol (5 mL) under nitrogen. The reaction was heated at 50° C. under nitrogen overnight. The reaction was then concentrated under vacuum. The residue was brought to pH 1 by the addition of 1 N aqueous HCl and this solution was then extracted with ethyl acetate three times (70 mL, 25 mL, 10 mL). The combined organic layer was dried (Na₂SO₄) and then concentrated under vacuum to yield the title compound as a colorless oil 2.04 g (99%) which was of sufficient purity to be used without purification. ¹H NMR (CD₃OD) δ 3.67 (q, 1H), 3.33 (s, 3H), and 1.33 ppm (d, 3H).

### Step 2: Preparation of 2-methoxypropanoyl chloride

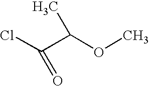

2-Methoxypropanoic acid (2.04 g, 19.2 mmol) was dissolved in dichloromethane (3 mL) which was stirred under nitrogen as a drop of dimethylformamide was added. Thionyl chloride was added dropwise into the reaction over 3 min and then the reaction was stirred at room temperature overnight. The reaction solution was concentrated in vacuo and the resulting pale yellow oil was placed under high vacuum to remove last traces of thionyl chloride. The yield of pure title compound was 303 mg (13%). ¹HNMR (CDCl₃) δ 4.10 (q, 1H), 3.48 (s, 3H), and 1.56 ppm (d, 3H).

### Step 3: Preparation of N-[4-(chloromethyl)pyridin-2-yl]-2-methoxypropanamide

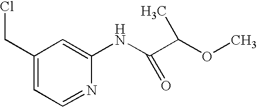

By using the methods described for preparation of Intermediate E (Step 2) and by substituting 2-methoxypropanoyl chloride instead of acetoxyacetyl chloride, Intermediate I was prepared from 352 mg of 4-(chloromethyl)pyridin-2-amine and proportional amounts of other reagents. The yield of pure title compound was 341 mg (60%) after silica gel chromatography using a gradient from 0-30% ethyl acetate in hexane.

¹H NMR (300 MHz, DMSO-d₆) δ 10.2 (bs, 1H), 8.30 (d, 1H), 8.17 (s, 1H), 7.16 (d, 1H), 4.77 (s, 2H), 4.00 (q, 1H), 3.26 (s, 3H), and 1.27 ppm (d, 3H).

### Intermediate J

### Preparation of N-[4-(chloromethyl)pyridin-2-yl]-2-methoxy-2-methylpropanamide

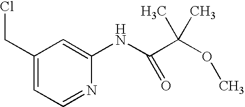

### Step 1: Preparation of 2-methoxy-2-methylpropanoic acid

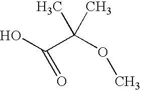

The procedure of Weizmann, Sulzbacher, and Bergmann as written in JACS 70, 1153 (1948), which is hereby incorporated by reference, was used as follows: A solution of potassium hydroxide (8.96 g, 159.7 mmol) in 5 mL of water and 20 mL of methanol was stirred with ice bath cooling under nitrogen as 1,1,1-trichloro-2-methylpropan-2-ol (7.10 g, 40.0 mmol) was carefully added dropwise over ten min. Vigorous bubbling was observed as a white precipitate formed. The ice bath was removed after 15 min. The reaction was stirred at room temperature for 2 h then refluxed for 3 h. The reaction was cooled to room temperature and the solids were then removed by filtration and rinsed with methanol (350 mL). The filtrate was concentrated under vacuum to remove methanol and the remaining aqueous layer was brought to pH 0 by the addition of aqueous HCl then extracted with ethyl acetate (300 mL). The extract was dried (Na₂SO₄) and concentrated in vacuo to yield 4.11 g of crude product, which was purified by vacuum distillation to yield 2.28 g (48%) of the pure title compound as a colorless oil which was distilled at 105° C. (28 mm Hg). ¹HNMR (CDCl₃) δ 9.65 (s, 1H), 3.20 (s, 3H) and 1.32 ppm (s, 3H).

### Step 2: Preparation of 2-methoxy-2-methylpropanoyl chloride

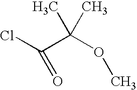

By following the procedure of Intermediate I (Step 2) but using 2-methoxy-2-methylpropanoic acid (6.99 g, 59.2 mmol) rather than 2-methoxypropanoic acid and proportional amounts of other reagents the title compound was synthesized. The crude product was distilled in vacuo to yield 2.671 g (33%) of pure compound, bp 44-48° C. (38 mm Hg).

¹HNMR (CDCl₃) δ 3.33 (s, 3H) and 1.51 ppm (s, 6H).

### Step 3: Preparation of N-[4-(chloromethyl)pyridin-2-yl]-2-methoxy-2-methylpropanamide

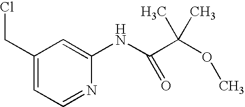

By using the methods described for preparation of Intermediate E (Step 2) and by substituting 2-methoxy-2-methylpropanoyl chloride instead of acetoxyacetyl chloride, Intermediate J was prepared from 1.04 g of 4-(chloromethyl)pyridin-2-amine and proportional amounts of other reagents. The yield of title compound was 1.23 g (69%) after silica gel chromatography using 30% ethyl acetate in hexane.

¹H NMR (300 MHz, DMSO-d₆) δ 9.41 (bs, 1H), 8.32 (d, 1H), 8.16 (s, 1H), 7.19 (d, 1H), 4.78 (s, 2H), 3.28 (s, 3H) and 1.36 ppm (s, 6H); ES-MS m/z 243.1 [M+H]⁺, HPLC RT (min) 2.12.

### Intermediate K

### Preparation of N-[4-(chloromethyl)pyridin-2-yl]methanesulfonamide

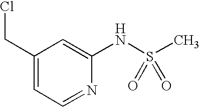

### Step 1: Preparation of N-[4-(chloromethyl)pyridin-2-yl]-N-(methylsulfonyl)methanesulfonamide

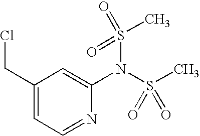

A solution of 4-(chloromethyl)pyridin-2-amine (500 mg, 3.51 mmol) and triethylamine (1.47 mL, 10.5 mmol) in ethyl acetate (4 mL) was stirred under nitrogen in a flask with ice bath cooling as methanesulfonyl chloride (0.81 mL, 10.5 mmol) was added dropwise. The reaction was then allowed to stir without cooling for 1 h before it was diluted with additional ethyl acetate, washed with water, dried (Na₂SO₄) and evaporated in vacuo. The resulting residue was purified by chromatography on silica gel using an ethyl acetate/hexane gradient to yield 860 mg (82%) of pure title compound.

¹H NMR (300 Hz, CD₂Cl₂) δ 8.56 (d, 1H), 7.50 (d, 1H), 7.41 (s, 1H), 4.66 (s, 2H), and 3.55 ppm (s, 6H); ES-MS m/z 299.0 [M+H]⁺, HPLC RT (min) 2.08.

### Step 2: Preparation of N-[4-(chloromethyl)pyridin-2-yl]methanesulfonamide

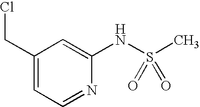

A suspension of N-[4-(chloromethyl)pyridin-2-yl]-N-(methylsulfonyl)-methanesulfonamide (700 mg, 2.34 mmol) in methanol (10 mL) and aqueous sodium hydroxide (1 N, 11.7 mL, 11.7 mmol) was stirred at ambient temperature as the starting material dissolved over 10 min. After another 10 min the reaction was adjusted to a pH between 3 and 6 by addition of aqueous hydrochloric acid (2 N) to precipitate the desired product as a white solid that was collected by filtration, washed with methanol and dried in vacuo. The yield of title compound was 250 mg (48%).

¹H NMR (300 MHz, DMSO-d₆) δ 10.93 (bs, 1H), 8.21 (d, 1H), 7.02 (m, 2H), 4.73 (s, 2H), and 3.23 ppm (s, 3H); ES-MS m/z 221.1 [M+H]⁺, HPLC RT (min) 1.45.

### Intermediate L

### Preparation of N-[4-(chloromethyl)pyridin-2-yl]-NA-ethylurea

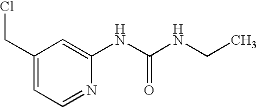

To 4-(chloromethyl)pyridin-2-amine (100 mg, 0.70 mmol) in 3 mL DMF was added ethyl isocyanate (59 mg, 0.84 mmol) and the resulting mixture was stirred under nitrogen for 16 h. The reaction was diluted with EtOAc (15 mL) and washed with H₂O three times, dried (Na₂SO₄) and evaporated in vacuo. The crude residue was purified by column chromatography on silica gel using 25% EtOAc in hexane to give 110 mg of N-[4-(chloromethyl)pyridin-2-yl]-N′-ethylurea (73%).

¹H NMR (DMSO-d₆) δ 9.22 (s, 1H), 8.14-8.16 (m, 1H), 7.91-7.94 (m, 1H), 7.45 (d, J=0.8 Hz, 1H), 6.93-6.95 (m, 1H), 4.70 (s, 2H), 3.12-3.14 (m, 2H), 1.01-1.09(m, 3H) ppm;

LCMS: 214.1 [M+H]⁺, RT 0.47 min.

### Intermediate M

### Preparation of N-[4-(chloromethyl)pyridin-2-yl]-NA-phenylurea

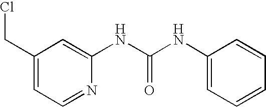

By using the methods described for preparation of Intermediate L and by substituting phenyl isocyanate instead of ethyl isocyanate, Intermediate M was prepared. From 250 mg of 4-(chloromethyl)pyridin-2-amine and proportional amounts of other reagents the yield of title compound was 218 mg (47%) after silica gel chromatography using a gradient from 0-40% ethyl acetate in hexane. Though there was evidence of contamination with the starting material 4-(chloromethyl)pyridin-2-amine in the NMR spectrum, this material was used without further purification and side products were separated by chromatography after the next step.

¹H NMR (300 MHz, DMSO-d₆) δ 10.25 (bs, 1H), 9.50 (bs, 1H), 8.29 (d, 1H), 7.95 (s, 1H), 7.52 (d, 1H), 7.27-7.36 (m, 2H), 7.0-7.1 (m, 2H), and 4.79 ppm (s, 2H); LCMS: 262.2 [M+H]⁺, RT 2.65 min.

### Intermediate N

### Preparation of N-[4-(chloromethyl)pyridin-2-yl]-N′-methylurea

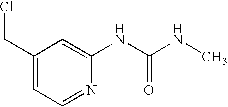

By using the methods described for preparation of Intermediate L and by substituting methyl isocyanate instead of ethyl isocyanate, Intermediate N was prepared. From 180 mg of 4-(chloromethyl)pyridin-2-amine and proportional amounts of other reagents the yield of pure title compound was 42 mg (17%) after silica gel chromatography using a gradient from 0-50% ethyl acetate in hexane followed by trituration of the residue with ether to remove a contaminant.

¹H NMR (DMSO-d₆) δ 9.31 (s, 1H), 8.16 (d, 1H), 7.92 (bm, 1H), 7.40 (s, 1H), 6.93 (d, 1H), 4.69 (s, 2H) and 2.70 ppm (d, 3H); LCMS: 200.1 [M+H]⁺, RT 1.17 min.

### Intermediate O

### Preparation of 2,4-dichloro-6-(chloromethyl)pyrimidine

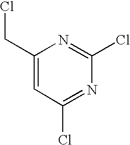

This product was prepared similarly to the 5-methyl substituted analog described in Biorg. Med. Chem. 2002, 10, 525, which is hereby incorporated by reference. A stirred suspension of 6-(chloromethyl)pyrimidine-2,4(1H,3H)-dione (5.2 g, 32.6 mmol) in POCl₃ (9.1 mL, 97.9 mmol) was refluxed for 16 h under nitrogen. The mixture was cooled and evaporated to leave a dark colored oil. Ice water was slowly added and the product was extracted into dichloromethane. The organic layer was washed with brine, dried over MgSO₄, and concentrated under reduced pressure to give 2,4-dichloro-6-(chloromethyl)pyrimidine (5 g) as a yellow oil. Though this product could be used in the next step with out purification, another batch prepared in the same way was further purified by chromatography to show the following NMR.

¹H NMR (DMSO-d₆) δ 7.90 (s, 1H) and 4.78 ppm (s, 2H).

### Intermediate P

### Preparation of 2-chloro-4-(chloromethyl)pyridine

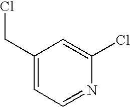

### Step 1: Preparation of (2-chloropyridin-4-yl)methanol

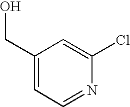

A sample of methyl 2-chloroisonicotinate (5.00 g, 29.14 mmol) was dissolved in 10 mL THF, treated with 10 drops of methanol, and cooled to 0° C. The solution was treated with lithium borohydride solution (21.86 mL of 1 M in THF, 43.71 mmol) and then allowed to warm to room temp. After 4 h the solution was cooled to 0° C. and quenched with 1 N HCl solution. The pH was adjusted to pH 10 with 1 N NaOH solution, and the reaction mixture was extracted with EtOAc. The organic extracts were washed with brine and concentrated in vacuo yielding 2.96 g (70.8%) of product.

¹H NMR (300 MHz, CD₃CN) δ 8.32 (d, 1H), 7.39 (s, 1H), 7.29 (d, 1H), 4.62 (s, 2H) and 3.53 ppm (bs, 1H).

### Step 2: Preparation of 2-chloro-4-(chloromethyl)pyridine

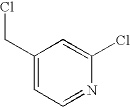

A sample of (2-chloropyridin-4-yl)methanol (110.0 mg, 0.77 mmol) was dissolved in anhydrous THF (1.5 mL), treated with N,N-diisopropylethylamine (0.29 mL, 1.69 mmol) and cooled to −78° C. Methanesulfonyl chloride was added (0.07 mL, 0.84 mmol), and the reaction mixture was allowed to slowly warm to room temperature overnight. The reaction mixture was then diluted with dichloromethane and washed with water. The organic layer was dried over Na₂SO₄ and concentrated in vacuo yielding the title compound (110.0 mg, 88.6%).

¹H NMR (300 MHz, CD₃CN) δ 8.40 (d, 1H), 7.49 (s, 1H), 7.39 (d, 1H) and 4.63 ppm (s, 2H); ES-MS m/z 183.2 [M+Na]⁺, HPLC RT (min) 2.30.

### Intermediate O

### Preparation of 4-(chloromethyl)-N-(4-methyl-1,3-thiazol-2-yl)pyridin-2-amine

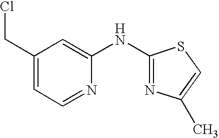

### Step 1: Preparation of 4-({[tert-butyl(dimethyl)silyl]oxy}methyl)pyridin-2-amine

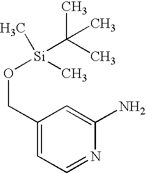

A solution of (2-aminopyridin-4-yl)methanol (5.0 g, 40 mmol), tert-butyldimethylsilyl chloride (6.07 g, 40 mmol), N-ethyl-N-isopropylpropan-2-amine (7.0 mL, 40 mmol) and N,N-dimethylpyridin-4-amine (0.49 g, 4 mmol) in dichloromethane (50 mL) was stirred 2 days at ambient temperature under nitrogen. The resulting reaction mixture was washed in sequence with aqueous sodium hydroxide (1 N), water and brine. It was then dried (Na₂SO₄) and concentrated in vacuo. The residue was chromatographed on silica gel using 50% ethyl acetate in hexane to yield pure title compound (5.47 g).

¹H NMR (300 MHz, CD₃CN) δ 7.75 (m, 1H), 6.39-6.48 (m, 2H), 4.70 (bs, 1H), 4. 50 (s, 2H), 0.83 (s, 9H) and 0.03 ppm (s, 6H); ES-MS m/z 239.3 [M+H]⁺, HPLC RT (min) 2.35.

### Step 2: Preparation of N-({[4-({[tert-butyl(dimethyl)silyl]oxy}methyl)pyridin-2-yl]amino}carbonothioyl)benzamide

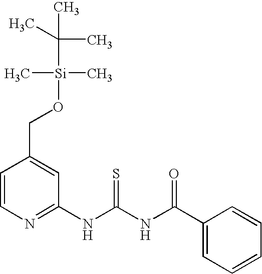

A solution of 4-({[tert-butyl(dimethyl)silyl]oxy}methyl)pyridin-2-amine (2.00 g, 8.39 mmol) and benzoyl isothiocyanate (1.51 g, 9.23 mmol) in toluene (20 mL) was heated to 85° C. for 12 h. The solvent was removed by evaporation in vacuo and the residue was purified by chromatography on silica gel using a gradient from 0-10% ethyl acetate in hexane to yield pure title compound as a yellow oil which solidified on standing (2.68 g, 79%).

¹H NMR (300 MHz, CD₃OD) δ 8.79 (bs, 1H), 8.18 (d, 1H), 7.83 (m, 2H), 7. 50 (m, 1H), 7.40 (m, 2H), 7.04 (m, 1H), 4.68 (s, 2H), 0.82 (s, 9H), and 0.03 ppm (s, 6H); ES-MS m/z 402.0 [M+H]⁺, HPLC RT (min) 4.24.

### Step 3: Preparation of N-[4-({[tert-butyl(dimethyl)silyl]oxy}methyl)pyridin-2-yl]thiourea

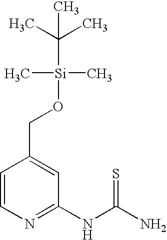

A solution of N-({[4-({[tert-butyl(dimethyl)silyl]oxy}methyl)pyridin-2-yl]amino}carbonothioyl)benzamide (1.00 g, 2.49 mmol) in absolute ethanol (15 mL) was stirred with potassium carbonate (0.344 g, 2.49 mmol) and heated to reflux under nitrogen for 16 h, after which the reaction mixture was filtered and the filtrate was evaporated under vacuum to give crude title compound (670 mg, >100%) as a white solid which was carried on to the next step without purification.

¹H NMR (300 MHz, DMSO-d₆) δ 10.55 (bs, 2H), 8.75 (bs, 1H), 8.05 (d, 1H), 7.10 (s, 1H), 6.83 (d, 1H), 4.60 (s, 2H), 0.83 (s, 9H) and 0.03 ppm (s, 6H); ES-MS m/z 298.2 [M+H]⁺, HPLC RT (min) 3.25.

### Step 4: Preparation of {2-[(4-methyl-1,3-thiazol-2-yl)amino]pyridin-4-yl}methanol

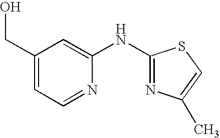

A solution of N-[4-({[tert-butyl(dimethyl)silyl]oxy}methyl)pyridin-2-yl]thiourea (crude material, 650 mg) and 1-chloroacetone (0.18 mL, 2.18 mmol) in ethanol (10 mL) was refluxed under nitrogen for 16 h and cooled. A white/pink solid was collected by filtration and washed with ethanol. The filtrate was evaporated in vacuo to yield a second white/pink solid. Comparison of the NMR of the two solids indicated that they were both the title compound and were pure enough (about 90%) to carry on to the next step without further purification (combined residue yield 516 mg, >100%).

¹H NMR (300 MHz, DMSO-d₆) δ 8.13 (d, 1H), 7.05 (s, 1H), 6.83 (d, 1H), 6.58 (s, 1H), 4.42 (s, 2H) and 2.18 ppm (s, 3H); ES-MS m/z 222.2 [M+H]⁺, HPLC RT (min) 1.41.

### Step 5: Preparation of 4-(chloromethyl)-N-(4-methyl-1,3-thiazol-2-yl)pyridin-2-amine

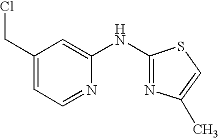

A mixture of {2-[(4-methyl-1,3-thiazol-2-yl)amino]pyridin-4-yl}methanol (200 mg, 0.9 mmol) and thionyl chloride (0.66 mL, 9.04 mmol) was stirred for 3 h and then evaporated in vacuo. The residue was dissolved in ethyl acetate and washed with saturated sodium bicarbonate. The aqueous layer was back extracted twice with ethyl acetate and then twice with a mixture of isopropanol, ethyl acetate and dichloromethane (1:8:1). The combined extracts were dried (Na₂SO₄) and concentrated in vacuo and the resulting residue was mixed with methanol, evaporated and then mixed with ethyl acetate and then evaporated again to yield the title compound as a light pink solid (200 mg, 92%) which was taken on to the next step as a crude solid.

¹H NMR (300 MHz, CD₂Cl₂) δ 8.30 (m, 1H), 6.98 (s, 1H), 6.90 (m, 1H), 6.50 (s, 1H), 4.55 (s, 2H) and 2.33 ppm (s, 3H); ES-MS m/z 240.2 [M+H]⁺, HPLC RT (min) 1.14.

### Intermediate R

### Preparation of N-({[4-(chloromethyl)pyridin-2-yl]amino}carbonyl)benzamide

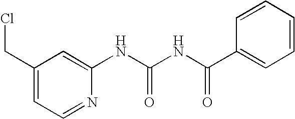

By using the methods described for preparation of Intermediate L and by substituting benzoyl isocyanate instead of ethyl isocyanate and using dichloromethane rather than DMF as solvent, Intermediate R was prepared. The product, which separated from the reaction mixture as a solid, was collected by filtration and washed with dichloromethane.

¹H NMR (DMSO-d₆) δ 11.01 (s, 1H), 10.98 (bs, 1H), 8.06 (d, 1H), 7.82 (s, 1H), 7.73 (d, 2H), 7.37 (t, 1H), 7.25 (t, 2H), 6.90 (d, 1H), and 4.52 (s, 2H).

### Preparation of Compounds of the Invention

### Example 1

### Preparation of 4-{[(5-{[(2,2-difluoro-1,3-benzodioxol-5-yl)amino]carbonyl}-1,3-thiazol-4-yl)amino]methyl}-N-methylpyridine-2-carboxamide

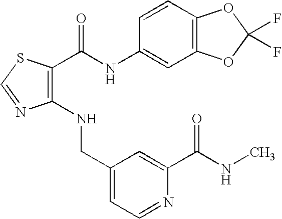

A solution of 4-amino-N-(2,2-difluoro-1,3-benzodioxol-5-yl)-1,3-thiazole-5-carboxamide (Intermediate A, 81.8 mg, 0.27 mmol), {2-[(methylamino)carbonyl]pyridin-4-yl}methyl methanesulfonate (Intermediate D, 66.8 mg, 0.27 mmol) and 2,6-di(tert)butyl-4-methylphenol (6 mg) in dry dimethylformamide (1.0 mL) was stirred under nitrogen as sodium iodide (41 mg, 0.27 mmol) was added. The resulting solution was stirred at 60° C. in a foil wrapped flask for 6 h and then left to stand overnight without heating. The resulting solution was diluted with 1 mL methanol and injected in two portions on a 150/20 mm C18 HPLC column using a gradient from 10-50% acetonitrile in water (plus 0.05% trifluoroacetic acid). The best fractions containing the desired material, as identified by LCMS, were combined, mixed with saturated NaHCO₃, and extracted three times with dichloromethane. The combined extracts were dried (Na₂SO₄) and evaporated in vacuo to yield 13.8 mg (11%) of pure title compound.

¹H NMR (300 MHz, CD₂Cl₂) δ 8.43 (s, 1H), 8.35 (bs, 1H), 8.00 (s, 1H), 7.93 (bs, 1H), 7.85 (t, 1H), 7.54 (s, 1H), 7.30 (bs, 1H), 7.08 (s, 1H), 6.93 (s, 2H), 4.75 (d, 2H) and 2.90 ppm (s, 3H); ES-MS m/z 448.3 [M+H]⁺, HPLC RT (min) 3.18.

### Example 2

### Preparation of N-methyl-4-{[(5-{[(2,2,3,3-tetrafluoro-2,3-dihydro-1,4-benzodioxin-6-yl)amino]carbonyl}-1,3-thiazol-4-yl)amino]methyl}pyridine-2-carboxamide

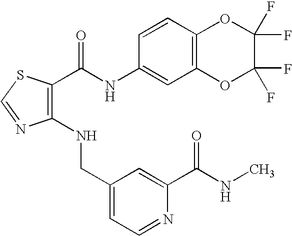

### Step 1: Preparation of methyl 4-[({2-[(methylamino)carbonyl]pyridin-4-yl}methyl]amino]-1,3-thiazole-5-carboxylate

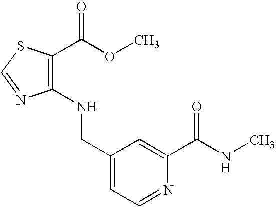

A solution of methyl 4-amino-1,3-thiazole-5-carboxylate (500 mg, 3.16 mmol) and {2-[(methylamino)carbonyl]pyridin-4-yl}methyl methanesulfonate (Intermediate D, 1.00 g, 4.11 mmol) in dry DMF (10 mL) was degassed by bubbling with nitrogen gas and then 2,6-di-tert-butyl-4-methylphenol (70 mg, 0.32 mmol) and sodium iodide (616 mg, 4.11 mmol) were added. The resulting mixture was heated with stirring under nitrogen in a foil wrapped flask at 70° C. for 5 h and then cooled, diluted with dichloromethane and washed with water. The organic was dried (Na₂SO₄) and evaporated in vacuo to give an oily residue which was purified by chromatography on silica gel using 50% ethyl acetate in hexane followed by 5% methanol in dichloromethane to give pure title compound (145 mg, 15%).

¹H NMR (300 MHz, DMSO-d₆) δ 8.94 (s, 1H), 8.71 (bq, 1H), 8.49 (d, 1H), 7.91 (s, 1H), 7.74 (t, 1H), 7.44 (d, 1H) 4.74 (d, 2H), 3.74 (s, 3H) and 2.78 ppm (d, 3H); ES-MS m/z 307.1 [M+H]⁺, HPLC RT (min) 2.30.

### Step 2: Preparation of 4-[({2-[(methylamino)carbonyl]pyridin-4-yl}methyl)amino]-1,3-thiazole-5-carboxylic acid

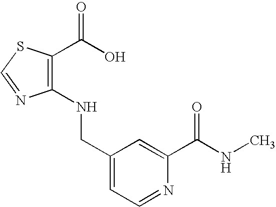

A solution of methyl 4-[({2-[(methylamino)carbonyl]pyridin-4-yl}methyl)amino]-1,3-thiazole-5-carboxylate (140 mg, 0.46 mmol) and aqueous sodium hydroxide (1 N, 4.57 mL) in ethanol (3 mL) and THF (3 mL) was stirred under nitrogen with heating at 70° C. for 1 h and then cooled. The resulting solution was condensed to a low volume by evaporation in vacuo and then diluted with aqueous monobasic potassium phosphate (1 N), adjusted to pH 4 by addition of aqueous hydrochloric acid (1 N) and extracted 5 times with ethyl acetate. Combined extracts were dried (Na₂SO₄) and evaporated in vacuo to yield the title compound (90 mg, 67%).

¹H NMR (300 MHz, CD₂Cl₂) δ 9.4 (broad, 1H), 8.52 (s, 1H), 8.33 (d, 1H), 8.1 (m, 2H), 8.02 (s, 1H), 7.29 (d, 1H), 4.75 (s, 2H) and 2.89 (d, 3H); ES-MS m/z 293.1 [M+H]⁺, HPLC RT (min) 1.90.

### Step 3: Preparation of N-methyl-4-{[(5-{[(2,2,3,3-tetrafluoro-2,3-dihydro-1,4-benzodioxin-6-yl)amino]carbonyl}-1,3-thiazol-4-yl)amino]methyl}pyridine-2-carboxamide

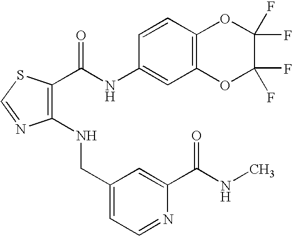

A mixture of 4-[({2-[(methylamino)carbonyl]pyridin-4-yl}methyl)amino]-1,3-thiazole-5-carboxylic acid (86 mg, 0.25 mmol), 2,2,3,3-tetrafluoro-2,3-dihydro-1,4-benzodioxin-6-amine (67 mg, 0.30 mmol), (benzotriazol-1-yloxy)tripyrrolidinophosphonium hexafluorophosphate (PyBOP, 156 mg, 0.30 mmol) and triethylamine (0.10 mL, 0.75 mmol) in DMF (1 mL) plus dichloromethane (3 mL) was heated with stirring under nitrogen at 60° C. for 24 h and cooled. HPLC analysis still showed considerable starting material remained, therefor additional 2,2,3,3-tetrafluoro-2,3-dihydro-1,4-benzodioxin-6-amine (56 mg, 0.25 mmol) was added and heating was continued another 4 h before the reaction mixture was injected directly on a preparative C18 HPLC system. Product containing fractions were evaporated in vacuo to remove some acetonitrile and then poured into saturated aqueous sodium bicarbonate and extracted with ethyl acetate. Combined extracts were dried (Na₂SO₄) and evaporated in vacuo to yield pure title compound (16 mg, 11%).

¹H NMR (300 MHz, CD₂Cl₂) δ 8.49 (s, 1H), 8.47 (s, 1H), 8.13 (s, 1H), 8.06 (m, 1H), 7.98 (t, 1H), 7.70 (d, 1H), 7.67 (s, 1H), 7.42 (dd, 1H), 7.28 (dd, 1H), 7.11 (d, 1H), 4.87 (d, 2H) and 2.98 (d, 3H); ES-MS m/z 498.1 [M+H]⁺, HPLC RT (min) 2.69.

### Example 3

### Preparation of 4-{[(5-{[(2,2-difluoro-1,3-benzodioxol-5-yl)amino]carbonyl}-1,3-thiazol-4-yl)amino]methyl}pyridine-2-carboxamide

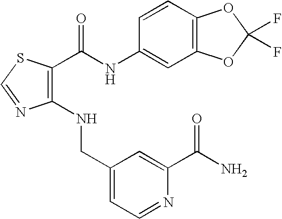

This material was prepared using the same method described for Example 1 but starting with 500 mg of 4-amino-N-(2,2-difluoro-1,3-benzodioxol-5-yl)-1,3-thiazole-5-carboxamide and proportionate amounts of Intermediate C rather than Intermediate D and also proportional amounts of the other reaction components. After heating at 60° C. in a foil wrapped flask for 3 h, an additional 115 mg of Intermediate D was added and stirring was continued at 60° C. for another 16 h before crude product was isolated as above. This material was purified by chromatography on silica gel using a gradient from 0-3% methanol in dichloromethane and then best fractions were re-purified on a second column using a gradient from 0-1% methanol. The yield of pure material was 264 mg (36%).

¹H NMR (300 MHz, CD₂Cl₂) δ 8.57 (s, 1H), 8.51 (d, 1H), 8.13 (s, 1H), 7.95 (t, 1H), 7.82 (bs, 1H), 7.75 (s, 1H), 7.46 (d, 1H), 7.20 (bs, 1H), 7.07 (s, 2H), 5.56 (bs, 1H) and 4.90 ppm (d, 2H); ES-MS m/z 434.1 [M+H]⁺, 456.1 [M+Na]⁺, HPLC RT (min) 3.32.

### Example 4

### Preparation of methyl 4-{[(5-{[(2,2-difluoro-1,3-benzodioxol-5-yl)amino]carbonyl}-1,3-thiazol-4-yl)amino]methyl}pyridine-2-carboxylate

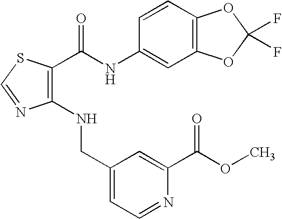

A suspension of 4-{[(5-{[(2,2-difluoro-1,3-benzodioxol-5-yl)amino]carbonyl}-1,3-thiazol-4-yl)amino]methyl}pyridine-2-carboxamide (35 mg, 0.06 mmol) in 1.2 mL methanol and N,N′-dimethylformamide dimethylacetal (23 mg, 0.19 mmol) was heated with stirring in a sealed vial at 50° C. The solid starting material dissolved in about 30 min to give a clear solution. After 2 h heating the solution was evaporated in vacuo and the residue was chromatographed on silica gel with a gradient from 0-1% methanol in dichloromethane to yield 10 mg (30%) of pure title compound.

¹H NMR (300 MHz, CD₂Cl₂) δ 8.65 (d, 1H), 8.55 (s, 1H), 8.07 (s, 1H), 7.94 (bt, 1H), 7.66 (s, 1H), 7.47 (d, 1H), 7.21 (bs, 1H), 7.06 (s, 2H) 4.87 (d, 2H) and 3.95 ppm (s, 3H); ES-MS m/z 449.2 [M+H]⁺, HPLC RT (min) 3.39.

### Example 5

### Preparation of 4-{[(5-{[(2,2-difluoro-1,3-benzodioxol-5-yl)amino]carbonyl}-1,3-thiazol-4-yl)amino]methyl}-N-(4-pyrrolidin-1-ylbutyl)pyridine-2-carboxamide

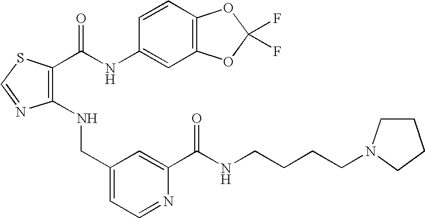

A slurry of 4-{[(5-{[(2,2-difluoro-1,3-benzodioxol-5-yl)amino]carbonyl}-1,3-thiazol-4-yl)amino]methyl}pyridine-2-carboxamide (95 mg, 0.21 mmol) in methanol (0.60 mL) was treated as in Example 4 to prepare a solution of methyl 4-{[(5-{[(2,2-difluoro-1,3-benzodioxol-5-yl)amino]carbonyl}-1,3-thiazol-4-yl)amino]methyl}pyridine-2-carboxylate, which was used directly by addition of 4-pyrrolidin-1-ylbutan-1-amine (266 mg, 1.87 mmol) and stirring 16 h. The reaction solution was purified by HPLC using direct injection, in three portions, on a YMC-Pack Pro C18 column (150×20 mm) and eluted at 20 mL/min with a gradient from 10-50% acetonitrile in water plus 0.05% TFA. Pure fractions from each injection were combined, made basic by addition of sodium bicarbonate and extracted with ethyl acetate. Combined extracts were dried (Na₂SO₄) and evaporated in vacuo to yield pure title compound (19 mg).

¹H NMR (300 MHz, CD₃OD) δ 8.48 (d, 1H), 8.07 (s, 1H), 8.05 (s, 1H), 7.50 (d, 1H), 7.2-7.27 (m, 2H), 7.08 (d, 1H), 4.81 (s, 2H), 3.39 (bt, 2H), 2.4-2.55 (m, 6H), 1.7-1.8 (m, 4H) and 1.5-1.65 ppm (m, 4H); ES-MS m/z 558.3 [M+H]⁺, and 580.3 [M+Na]⁺, HPLC RT (min) 2.90.

### Example 6

### Preparation of 4-{[(5-{[(2,2-difluoro-1,3-benzodioxol-5-yl)amino]carbonyl}-1,3-thiazol-4-yl)amino]methyl}-N-[4-(dimethylamino)butyl]pyridine-2-carboxamide

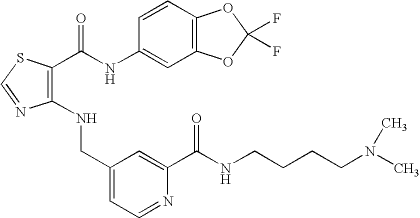

A solution of methyl 4-{[(5-{[(2,2-difluoro-1,3-benzodioxol-5-yl)amino]carbonyl}-1,3-thiazol-4-yl)amino]methyl}pyridine-2-carboxylate (50 mg, 0.11 mmol) in methanol was prepared as in Example 4 and this solution was used directly by addition of N,N-dimethylbutane-1,4-diamine (117 mg, 1.0 mmol), followed by stirring for 3 h at 50° C. in a sealed tube. HPLC (gradient 10-50% acetonitrile in water plus 0.05% TFA) of an aliquot showed residual starting ester so additional N,N-dimethylbutane-1,4-diamine (37 mg, 0.3 mmol) was added and heating was continued for another 1 h. The crude product solution was purified as in Example 5 to give 7 mg of title compound.

¹H NMR (300 MHz, CD₃OD) δ 8.48 (d, 1H), 8.10 (s, 1H), 8.06 (s, 1H), 7.49 (d, 1H), 7.33 (d, meta coupling, 1H), 7.28 (d, ortho coupling, 1H), 7.08 (dd, ortho and meta coupling, 1H), 4.80 (s, 2H), 4.10 (bt, 1H), 3.41 (bt, 2H), 2.3-2.4 (m, 2H), 2.23 (s, 6H) and 1.5-1.7 ppm (m, 4H); ES-MS m/z 533.2 [M+H]⁺, and 580.3 [M+Na]⁺, HPLC RT (min) 2.53.

### Example 7

### Preparation of N-(2,2-difluoro-1,3-benzodioxol-5-yl)-4-[([2-[(methoxyacetyl)amino]pyridin-4-yl]methyl)amino]-1,3-thiazole-5-carboxamide

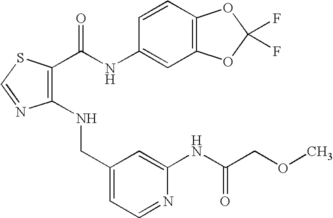

This material was prepared using the same method described for Example 1 but starting with 116 mg of 4-amino-N-(2,2-difluoro-1,3-benzodioxol-5-yl)-1,3-thiazole-5-carboxamide (Intermediate A) and proportionate amounts of Intermediate G rather than Intermediate D and also proportional amounts of the other reaction components. The reaction mixture was heated at 60° C. in a foil wrapped flask overnight before crude product was isolated as above. This material was purified by chromatography on silica gel using a gradient from 0-60% ethyl acetate in hexane. The yield of pure material was 28 mg (15%).

¹H NMR (300 MHz, acetonitrile-d₃) δ 8.72 (bs, 1H), 8.70 (s, 1H), 8.21 (d, 1H), 8.14 (s, 1H), 8.08 (bs, 1H), 7.96 (bt, 1H), 7.67 (d, 1H, meta coupling), 7.28 (dd, 1H, meta and ortho coupling), 7.16 (d, 1H), 7.08 (d, 1H), 4.80 (d, 2H), 3.99 (s, 2H) and 3.46 ppm (s, 3H); ES-MS m/z 478.1 [M+H]⁺, 500.1 [M+Na]⁺, HPLC RT (min) 3.40.

### Example 8

### Preparation of 4-[({2-[(methoxyacetyl)amino]pyridin-4-yl}methyl)amino]-N-(2,2,3,3-tetrafluoro-2,3-dihydro-1,4-benzodioxin-6-yl)-1,3-thiazole-5-carboxamide

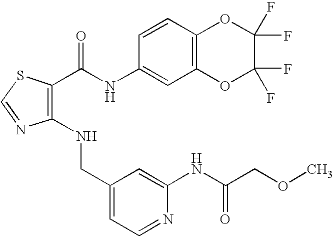

The title compound was prepared using the same method described for Example 1 but starting with 118 mg of 4-amino-N-(2,2,3,3-tetrafluoro-2,3-dihydro-1,4-benzodioxin-6-yl)-1,3-thiazole-5-carboxamide (Intermediate B) and proportionate amounts of Intermediate G rather than Intermediate D and also proportional amounts of the other reaction components. The reaction mixture was heated at 60° C. in a foil wrapped flask for 19 h before crude product was isolated by evaporation of solvents in vacuo. This residue was purified by preparative C18 HPLC as a solution in methanol and a gradient from 10-60% acetonitrile in water plus 0.05% TFA. Product containing fractions were diluted with saturated aqueous NaHCO₃ and extracted with dichloromethane. The extracts were dried (Na₂SO₄) and evaporated in vacuo to yield 35 mg (20%) of the pure title compound as a solid.

¹H NMR (300 MHz, acetonitrile-d₃) δ 8.73 (bs, 1H), 8.70 (s, 1H), 8.21 (d, 1H), 8.14 (s, 1H), 8.12 (bs, 1H), 7.99 (bt, 1H), 7.74 (d, 1H, meta coupling), 7.47 (dd, 1H, meta and ortho coupling), 7.26 (d, 1H), 7.08 (d, 1), 4.81 (d, 2H), 4.00 (s, 2H) and 3.47 ppm (s, 3H); ES-MS m/z 528.3 [M+H]⁺, 550.1 [M+Na]⁺, HPLC RT (min) 3.40.

### Example 9

### Preparation of 4-{[(2-{[(2-methoxyethoxy)acetyl]amino}pyridin-4-yl)methyl]amino}-N-(2,2,3,3-tetrafluoro-2,3-dihydro-1,4-benzodioxin-6-yl)-1,3-thiazole-5-carboxamide

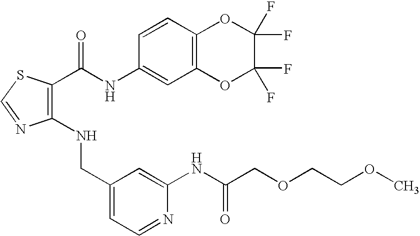

The title compound was prepared using the same method described for Example 1 but starting with 117 mg of 4-amino-N-(2,2,3,3-tetrafluoro-2,3-dihydro-1,4-benzodioxin-6-yl)-1,3-thiazole-5-carboxamide (Intermediate B) and proportionate amounts of Intermediate H rather than Intermediate D and also proportional amounts of the other reaction components. The reaction mixture was heated at 60° C. in a foil wrapped flask 19 h before the crude product was isolated by evaporation of solvents in vacuo. This residue was purified by preparative C18 HPLC as a solution in methanol and a gradient from 10-60% acetonitrile in water plus 0.05% TFA. Product containing fractions were diluted with saturated aqueous NaHCO₃ and extracted with dichloromethane. The extracts were dried (Na₂SO₄) and evaporated in vacuo to yield 35 mg (18%) of the pure title compound as a solid.

¹H NMR (300 MHz, DMSO-d₆) δ 9.86 (s, 1H), 9.81 (s, 1H), 8.94 (s, 1H), 8.20 (d, 1H), 8.10 (t, 1H), 8.05 (s, 1H), 7.87 (s, 1H), 7.54 (d, 1H), 7.41 (d, 1H), 7.02 (d, 1H), 4.71 (d, 2H), 4.10 (s, 2H), 3.65 (t, 2H), 3.48 (t, 2H) and 3.27 ppm (s, 3H); ES-MS m/z 572.1 [M+H]⁺, 594.1 [M+Na]⁺, HPLC RT (min) 3.01.

### Example 10

### Preparation of 4-([{2-(acetylamino)pyridin-4-yl]methyl}amino)-N-(2,2-difluoro-1,3-benzodioxol-5-yl)-1,3-thiazole-5-carboxamide

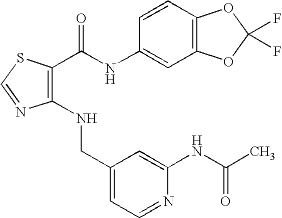

The title compound was prepared using the same method described for Example 1 but starting with 50 mg of 4-amino-N-(2,2-difluoro-1,3-benzodioxol-5-yl)-1,3-thiazole-5-carboxamide (Intermediate A) and proportionate amounts of Intermediate F rather than Intermediate D and also proportional amounts of the other reaction components. The reaction mixture was heated at 60° C. in a foil wrapped flask 5 h and then left to stand without heating overnight before the crude product was isolated as above. This residue was purified by preparative HPLC as in example 4 to yield 10 mg of pure title compound.

¹H NMR (300 MHz, CD₂Cl₂) δ 8.58 (s, 1H), 8.17 (d, 1H), 8.15 (s, 1H), 8.09 (bs, 1H), 7.89 (bt, 1H), 7.67 (d, 1H, meta coupling), 7.14 (s, 1H), 7.00-7.07 (m, 3H), 4.80 (d, 2H) and 2.13 ppm (s, 3H); ES-MS m/z 448.2 [M+H]⁺, 470.2[M+Na]⁺, HPLC RT (min) 2.93.

### Example 11

### Preparation of 4-({[2-(acetylamino)pyridin-4-yl]methyl}amino)-N-(2,2,3,3-tetrafluoro-2,3-dihydro-1,4-benzodioxin-6-yl)-1,3-thiazole-5-carboxamide

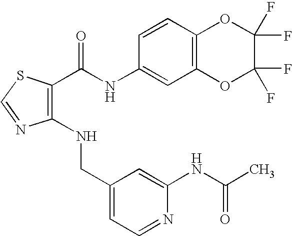

The title compound was prepared using the same method described for Example 1 but starting with 50 mg of 4-amino-N-(2,2,3,3-tetrafluoro-2,3-dihydro-1,4-benzodioxin-6-yl)-1,3-thiazole-5-carboxamide (Intermediate B) rather than 4-amino-N-(2,2-difluoro-1,3-benzodioxol-5-yl)-1,3-thiazole-5-carboxamide and proportionate amounts of Intermediate F rather than Intermediate D and also proportional amounts of the other reaction components. The reaction mixture was heated at 60° C. in a foil wrapped flask for 16 h and cooled. The reaction mixture was diluted with ethyl acetate, washed with water, dried (Na₂SO₄) and evaporated in vacuo. The crude product was purified by preparative HPLC to then yield pure title compound (5 mg).

¹H NMR (300 MHz, CD₂Cl₂) δ 8.59 (s, 1H), 8.17 (m, 2H), 8.06 (bs, 1H), 7.89 (bt, 1H), 7.68 (d, 1H, meta coupling), 7.17-7.27 (m, 2H), 7.12 (d, 1H), 7.00 (d, 1H), 4.80 (d, 2H) and 2.13 ppm (s, 3H); ES-MS m/z 498.2 [M+H]⁺, 520.1 [M+Na]⁺, HPLC RT (min) 2.79.

### Example 12

### Preparation of 4-[({2-[(methylsulfonyl)amino]pyridin-4-yl}methyl)amino]-N-(2,2,3,3-tetrafluoro-2,3-dihydro-1,4-benzodioxin-6-yl)-1,3-thiazole-5-carboxamide

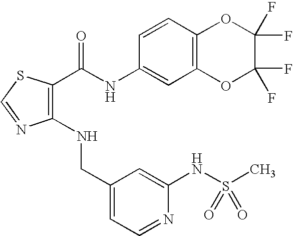

The title compound was prepared using the same method described for Example 1 but starting with 118 mg of 4-amino-N-(2,2,3,3-tetrafluoro-2,3-dihydro-1,4-benzodioxin-6-yl)-1,3-thiazole-5-carboxamide (Intermediate B) rather than 4-amino-N-(2,2-difluoro-1,3-benzodioxol-5-yl)-1,3-thiazole-5-carboxamide and proportionate amounts of N-[4-(chloromethyl)pyridin-2-yl]methanesulfonamide (Intermediate K) rather than Intermediate D and also proportional amounts of the other reaction components. The reaction mixture was heated at 60° C. in a foil wrapped flask 16 h and cooled. The reaction mixture was diluted with ethyl acetate, washed with water, dried (Na₂SO₄) and evaporated in vacuo. The crude product was purified by preparative HPLC to yield pure title compound (8.3 mg).

¹H NMR (300 MHz, CD₃OD) δ 8.78 (s, 1H), 7.95 (d, 1H), 7.79 (m, 1H), 7.74 (s, 1H), 7.47 (m, 1H), 7.23 (dd, 1H), 7.08 (s, 1H), 6.92 (d, 1H), 4.78 (s, 2H) and 3.11 ppm (s, 3H); ES-MS m/z 534.1 [M+H]⁺, HPLC RT (min) 3.07.

### Example 13

### Preparation of 2-[(4-{[(5-{[(2,2-difluoro-1,3-benzodioxol-5-yl)amino]carbonyl}-1,3-thiazol-4-yl)amino]methyl}pyridin-2-yl)amino]-2-oxoethyl acetate

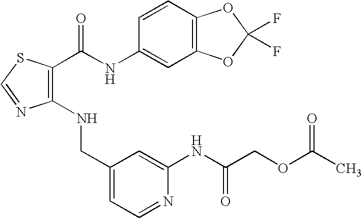

The title compound was prepared using the same method described for Example 1 but starting with 110 mg of 4-amino-N-(2,2-difluoro-1,3-benzodioxol-5-yl)-1,3-thiazole-5-carboxamide (Intermediate A) and proportionate amounts of 2-{[4-(chloromethyl)pyridin-2-yl]amino}-2-oxoethyl acetate (Intermediate E) rather than Intermediate D and also proportional amounts of the other reaction components. The reaction mixture was heated at 60° C. in a foil wrapped flask overnight before crude product was isolated as above in Example 12. This residue was purified by chromatography on silica gel using a gradient from 10-60% ethyl acetate in hexane. The yield of pure title compound was 38 mg (20%).

¹H NMR (300 MHz, CD₂Cl₂) δ 8.64 (bs, 1H), 8.57 (s, 1H), 8.20 (d, 1H), 8.15 (s, 1H), 7.91 (bt, 1H), 7.63 (d, 1H, meta coupling), 7.45 (s, 1H), 7.00-7.12 (m, 3H), 4.83 (d, 2H), 4.67 (s, 2H) and 2.20 ppm (s, 3H); ES-MS m/z 506.1 [M+H]⁺, 528.1 [M+Na]⁺, HPLC RT (min) 2.71.

### Example 14

### Preparation of 2-oxo-2-[(4-{[(5-{[(2,2,3,3-tetrafluoro-2,3-dihydro-1,4-benzodioxin-6-yl)amino]carbonyl}-1,3-thiazol-4-yl)amino]methyl}pyridin-2-yl)amino]ethyl acetate

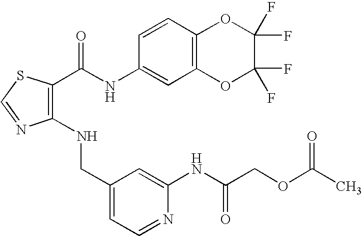

The title compound was prepared using the same method described for Example 1 but starting with 118 mg of 4-amino-N-(2,2,3,3-tetrafluoro-2,3-dihydro-1,4-benzodioxin-6-yl)-1,3-thiazole-5-carboxamide (Intermediate B) rather than 4-amino-N-(2,2-difluoro-1,3-benzodioxol-5-yl)-1,3-thiazole-5-carboxamide and proportionate amounts of 2-{[4-(chloromethyl)pyridin-2-yl]amino}-2-oxoethyl acetate (Intermediate E) rather than Intermediate D and also proportional amounts of the other reaction components. The reaction mixture was heated at 60° C. in a foil wrapped flask for 16 h and cooled. The reaction mixture was diluted with ethyl acetate, washed with water, dried (Na₂SO₄) and evaporated in vacuo. The crude residue was purified by chromatography on silica gel using a gradient from 10-60% ethyl acetate in hexane. The yield of title compound was 35 mg (19%).

¹H NMR (300 MHz, CD₂Cl₂) δ 8.93 (bs, 1H), 8.60 (s, 1H), 8.26 (bs, 1H), 8.22 (d, 1H), 7.93 (bt, 1H), 7.70 (d, 1H), 7.10-7.28 (m, 4H), 4.86 (m, 2H), 4.68 (s, 2H) and 2.21 ppm (s, 3H); ES-MS m/z 556.0 [M+H]⁺, 578.0 [M+Na]⁺, HPLC RT (min) 3.08.

### Example 15

### Preparation of 4-{[(2-aminopyridin-4-yl)methyl]amino}-N-(2,2-difluoro-1,3-benzodioxol-5-yl)-1,3-thiazole-5-carboxamide

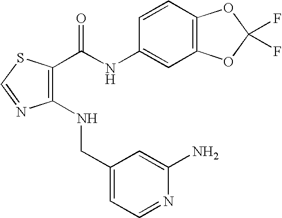

A solution of 2-[(4-{[(5-{[(2,2-difluoro-1,3-benzodioxol-5-yl)amino]carbonyl}-1,3-thiazol-4-yl)amino]methyl}pyridin-2-yl)amino]-2-oxoethyl acetate (Example 13, 17 mg) in methanol (1 mL) plus dichloromethane (0.2 mL) was stirred with 43 mg of potassium carbonate overnight to yield a mixture of the title compound and N-(2,2-difluoro-1,3-benzodioxol-5-yl)-4-({[2-(glycoloylamino)pyridin-4-yl]methyl}amino)-1,3-thiazole-5-carboxamide. This mixture was filtered and evaporated in vacuo and the residue was mixed with aqueous sodium hydroxide (0.3 mL, 1 N), THF (3 mL) and methanol (0.3 mL) and stirred 3 days. Solvent was removed by evaporation in vacuo and the residue was extracted with ethyl acetate. The extract was dried (Na₂SO₄) and evaporated in vacuo to yield pure title compound (10 mg).

¹H NMR (300 MHz, CD₂Cl₂) δ 8.50 (s, 1H), 7.86 (d, 1H), 7.75 (bt, 1H), 7.57 (d, 1H, meta coupling), 7.05 (s, 1H), 6.96 (s, 2H), 6.50 (d, 1H), 6.36 (s, 1H), 4.60 (d, 2H) and 4.38 (bs, 2H); ES-MS m/z 406.4 [M+H]⁺, HPLC RT (min) 2.67.

### Example 16

### Preparation of 4-{[(2-aminopyridin-4-yl)methyl]amino}-N-(2,2,3,3-tetrafluoro-2,3-dihydro-1,4-benzodioxin-6-yl)-1,3-thiazole-5-carboxamide

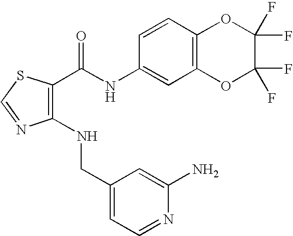

A solution of 2-oxo-2-[(4-{[(5-{[(2,2,3,3-tetrafluoro-2,3-dihydro-1,4-benzodioxin-6-yl)amino]carbonyl}-1,3-thiazol-4-yl)amino]methyl}pyridin-2-yl)amino]ethyl acetate (Example 14, 14 mg) in methanol (1 mL) plus dichloromethane (0.2 mL) was stirred with 31 mg of potassium carbonate 2 h to yield a mixture of products in which the title compound was the major component. This mixture was diluted with dichloromethane and filtered. After this solution stood overnight, evaluation by HPLC indicated that the only significant product was the title compound in a reasonably pure form (>90%). The extract was dried (Na₂SO₄) and evaporated in vacuo to yield pure title compound (7 mg).

¹H NMR (300 MHz, CD₃OD) δ 8.79 (s, 1H), 7.78 (m, 2H), 7.47 (dd, 1H), 7.23 (d, 1H), 7.55 (m, 2H) and 4.68 (s, 2H); ES-MS m/z 456.0 [M+H]⁺, HPLC RT (min) 2.78.

### Example 17

### Preparation of N-(2,2-difluoro-1,3-benzodioxol-5-yl)-4-{[(2-{[(ethylamino)carbonyl]amino}pyridin-4-yl)methyl]amino}-1,3-thiazole-5-carboxamide

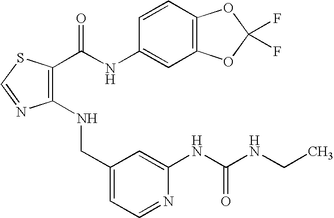

The title compound was prepared using the same method described for Example 1 but starting with 90 mg of 4-amino-N-(2,2-difluoro-1,3-benzodioxol-5-yl)-1,3-thiazole-5-carboxamide (Intermediate A) and proportionate amounts of Intermediate L rather than Intermediate D and also proportional amounts of the other reaction components. The reaction mixture was heated at 60° C. in a foil wrapped flask for 40 h. The resulting final crude mixture was diluted with saturated aqueous sodium bicarbonate and extracted 3 times with ethyl acetate. The combined extracts were dried (Na₂SO₄) and evaporated in vacuo to yield a residue that was purified by preparative C18 HPLC by injecting a methanol solution and eluting with a gradient from 10-60% acetonitrile in water plus 0.05% TFA. The free base was prepared from the TFA salt by addition of saturated aqueous NaHCO₃ to the fractions containing the product and extracting with dichloromethane followed by drying (Na₂SO₄) concentration of the extract in vacuo to yield pure title compound. The yield of title compound was 18.6 mg.

¹H NMR (300 MHz, DMSO-d₆) δ 9.80 (s, 1H), 9.15 (bs, 1H), 8.98 (s, 1H), 8.26 (bs, 1H), 8.05 (m, 2H), 7.83 (s, 1H), 7.34-7.44 (m, 2H), 7.26 (s, 1H), 6.85 (d, 1H), 4.66 (d, 2H), 3.16 (q, 2H), and 1.08 ppm (t, 3H); ES-MS m/z 477.45 [M+H]⁺, HPLC RT (min) 3.03.

### Example 18

### Preparation of N-(2,2-difluoro-1,3-benzodioxol-5-yl)-4-{[(2-{[(methylamino)carbonyl]amino}pyridin-4-yl)methyl]amino}-1,3-thiazole-5-carboxamide

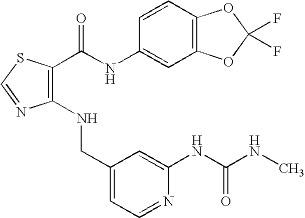

The title compound was prepared using the same method described for Example 1 but starting with 95 mg of 4-amino-N-(2,2-difluoro-1,3-benzodioxol-5-yl)-1,3-thiazole-5-carboxamide (Intermediate A) and proportionate amounts of Intermediate N rather than Intermediate D and also proportional amounts of the other reaction components. The reaction mixture was heated at 60° C. in a foil wrapped flask for 19 h until an LCMS analysis of the reaction mixture showed substantial conversion to products. The resulting final crude mixture was evaporated at reduced pressure with warming to remove the DMF. The residue was diluted with 2 mL of methanol and purified by preparative C18 HPLC using water to acetonitrile gradient (10-60% plus 0.05% TFA). The fractions containing the product were converted to free base by addition of saturated aqueous NaHCO₃ and extraction with dichloromethane four times. The combined extracts were washed with saturated brine, dried (Na₂SO₄) and evaporated in vacuo to yield pure title compound (36 mg, 25% theory).

¹H NMR (300 MHz, DMSO-d₆) δ 9.77 (s, 1H), 9.18 (s, 1H), 8.93 (s, 1H), 8.21 (bs, 1H), 8.02 (m, 2H), 7.79 (s, 1H), 7.32-7.42 (m, 2H), 7.16 (s, 1H), 6.81 (d, 1H), 4.62 (d, 2H) and 2.67 ppm (d, 3H); ES-MS m/z 463.1 [M+H]⁺, HPLC RT (min) 2.48.

### Example 19

### Preparation of 4-[({2-[(anilinocarbonyl)amino]pyridin-4-yl}methyl)amino]-N-(2,2-difluoro-1,3-benzodioxol-5-yl)-1,3-thiazole-5-carboxamide

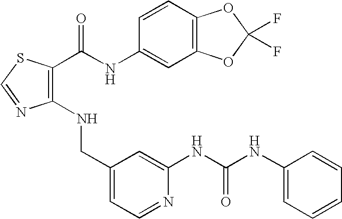

The title compound is prepared using the same method described for Example 1 but starting with 4-amino-N-(2,2-difluoro-1,3-benzodioxol-5-yl)-1,3-thiazole-5-carboxamide (Intermediate A) and proportionate amounts of Intermediate M rather than Intermediate D and also proportional amounts of the other reaction components. The reaction mixture is heated at 60° C. in a foil wrapped flask for between 2 and 24 h until an LCMS analysis of the reaction mixture shows substantial conversion to products. The resulting final crude mixture is diluted with saturated aqueous sodium bicarbonate and extracted 3 times with ethyl acetate. The combined extracts are dried (Na₂SO₄) and evaporated in vacuo to yield a residue that is purified by preparative C18 HPLC using water to acetonitrile gradient (usually 10-50%) with added 0.05-0.1% TFA. The free base is prepared from the TFA salt by addition of saturated aqueous NaHCO₃ to the fractions containing the product and extraction with dichloromethane followed by drying of the extract (Na₂SO₄) and concentration in vacuo to yield pure title compound.

### Example 20

### Preparation of 4-{[(2-{[(methylamino)carbonyl]amino}pyridin-4-yl)methyl]amino}-N-(2,2,3,3-tetrafluoro-2,3-dihydro-1,4-benzodioxin-6-yl)-1,3-thiazole-5-carboxamide

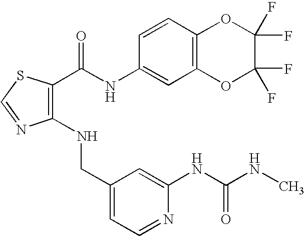

The title compound was prepared using the same method described for Example 1 but starting with 88 mg of 4-amino-N-(2,2,3,3-tetrafluoro-2,3-dihydro-1,4-benzodioxin-6-yl)-1,3-thiazole-5-carboxamide (Intermediate B) and proportionate amounts of Intermediate N rather than Intermediate D and also proportional amounts of the other reaction components. The reaction mixture was heated at 60° C. in a foil wrapped flask for 17 h until an LCMS analysis of the reaction mixture showed substantial conversion to products. The resultant final crude mixture was evaporated at reduced pressure with warming to remove the DMF. The residue was diluted with 2 mL of methanol and purified by preparative C18 HPLC using water to acetonitrile gradient (10-60% plus 0.05% TFA). The fractions containing the product were converted to free base by addition of saturated aqueous NaHCO₃ and extraction with dichloromethane three times followed by drying (Na₂SO₄) and concentration in vacuo to yield pure title compound (32 mg, 25% theory).

¹H NMR (300 MHz, DMSO-d₆) δ 9.85 (s, 1H), 9.17 (s, 1H), 8.93 (s, 1H), 8.21 (bs, 1H), 8.05 (m, 2H), 7.86 (s, 1H), 7.53 (dd, 1H), 7.40 (d, 1H), 7.16 (s, 1H), 6.80 (d, 1H), 4.64 (d, 2H) and 2.68 ppm (d, 3H); ES-MS m/z 513.2 [M+H]⁺, HPLC RT (min) 2.80.

### Example 21

### Preparation of 4-{[(2-{[(ethylamino)carbonyl]amino}pyridin-4-yl)methyl]amino}-N-(2,2,3,3-tetrafluoro-2,3-dihydro-1,4-benzodioxin-6-yl)-1,3-thiazole-5-carboxamide

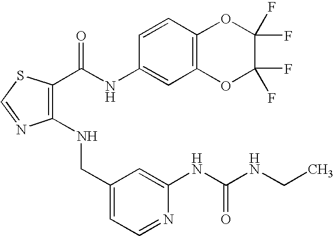

The title compound was prepared using the same method described for Example I but starting with 93 mg of 4-amino-N-(2,2,3,3-tetrafluoro-2,3-dihydro-1,4-benzodioxin-6-yl)-1,3-thiazole-5-carboxamide (Intermediate B) and proportionate amounts of Intermediate L rather than Intermediate D and also proportional amounts of the other reaction components. The reaction mixture was heated at 60° C. in a foil wrapped flask for 17 h until an LCMS analysis of the reaction mixture showed substantial conversion to products. The resultant final crude mixture was evaporated at reduced pressure with warming to remove the DMF. The residue was diluted with 2 mL of methanol and purified by preparative C18 HPLC using water to acetonitrile gradient (10-60% plus 0.05% TFA). The fractions containing the product were converted to free base by addition of saturated aqueous NaHCO₃ and extraction with dichloromethane three times followed by drying (Na₂SO₄) and concentration in vacuo to yield pure title compound (44 mg, 31% theory).

¹H NMR (300 MHz, DMSO-d₆) δ 9.87 (s, 1H), 9.12 (s, 1H), 8.95 (s, 1H), 8.22 (bs, 1H), 8.07 (m, 2H), 7.86 (s, 1H), 7.54 (dd, 1H), 7.42 (d, 1H), 7.21 (s, 1H), 6.81 (d, 1H), 4.64 (d, 2H) 3.17 (q, 2H) and 1.07 ppm (t, 3H); ES-MS m/z 527.2 [M+H]⁺,HPLC RT (min) 2.90.

### Example 21-A

### Preparation of N-(2,2-difluoro-1,3-benzodioxol-5-yl)-4-{[(2-{methyl](methyl amino)carbonyl]amino}pyridin-4-yl)methyl]amino}-1,3-thiazole-5-carboxamide

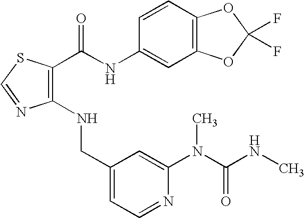

### Step 1: Preparation of [2-(methylamino)pyridin-4-yl]methanol

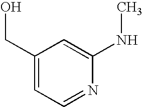

A solution of (2-chloropyridine-4-yl)methanol (from the preparation of Intermediate P, Step 1) and methylamine hydrochloride in pyridine is heated at 200° C. in a sealed tube for about 16 h. The solvent is removed by evaporation in vacuo and the crude product residue is purified by chromatography on silica gel using a gradient from dichloromethane to about 10% methanol in dichloromethane.

### Step 2 Preparation of 4-(chloromethyl)-N-methylpyridin-2-amine

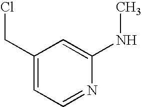

By using the general method of preparation of Intermediate E, Step 1 but substituting [2-(methylamino)pyridin-4-yl]methanol for (2-aminopyridin-4-yl)methanol, the Title compound is prepared.

### Step 3. Preparation of N-[4-(chloromethyl)pyridin-2-yl]-N,N′-dimethylurea

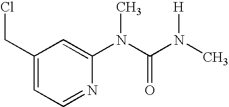

The step is carried out using the method described for the preparation of Intermediate L but using the product of Step 2 above rather than 4-(chloromethyl)pyridin-2-amine and methyl isocyanate rather than ethyl isocyanate.

### Step 4: Preparation of the Title Compound

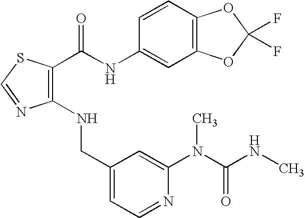

The title compound can be prepared using the same method described for Example 1 but starting with 4-amino-N-(2,2-difluoro-1,3-benzodioxol-5-yl)-1,3-thiazole-5-carboxamide (Intermediate A) and a proportionate amount of N-[4-(chloromethyl)pyridin-2-yl]-N,N′-dimethylurea rather than Intermediate D. The reaction mixture is heated at 60° C. in a foil wrapped flask under nitrogen for between 2 and 24 h until an LCMS analysis of the reaction mixture shows substantial conversion to products. The resulting final crude mixture is diluted with saturated aqueous sodium bicarbonate and extracted 3 times with ethyl acetate. The combined extracts are dried (Na₂SO₄) and evaporated in vacuo to yield a residue that is purified by preparative C18 HPLC using water to acetonitrile gradient (usually 10-50%) with added 0.05-0.1% TFA. The free base is prepared from the TFA salt by addition of saturated aqueous NaHCO₃ to the fractions containing the product and extraction with dichloromethane followed by drying of the extract (Na₂SO₄) and concentration in vacuo to yield pure title compound.

### Example 21-o

### Preparation of 4-{[(2-{[(methylamino)carbonyl]amino}pyridin-4-yl)methyl]amino}-N-(2,2,4,4-tetrafluoro-4H-1,3-benzodioxin-6-yl)-1,3-thiazole-5-carboxamide

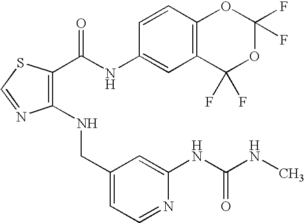

The title compound was prepared using the same method described for Example 1 but starting with 4-amino-N-(2,2,4,4-tetrafluoro-4H-1,3-benzodioxin-6-yl)-1,3-thiazole-5-carboxamide (Intermediate B-2) rather than Intermediate A and proportionate amounts of Intermediate N rather than Intermediate D and also proportional amounts of the other reaction components. The reaction mixture was heated at 60° C. in a foil wrapped flask for 24 h. The resulting final crude mixture was diluted with saturated aqueous sodium bicarbonate and extracted with ethyl acetate. The extract was washed 3 times with water and then with saturated brine. The extract was dried (Na₂SO₄) and evaporated in vacuo to yield a residue that was purified by chromatography on silica gel using a gradient from 0-100% ethyl acetate in hexane.

¹H NMR (300 MHz, DMSO-d₆) δ 9.96 (s, 1H), 9.20 (s, 1H), 8.95 (s, 1H), 8.33 (s, 1H), 8.22 (m, 1H), 8.11 (t, 1H), 8.05 (d, 1H), 7.96 (d, 1H), 7.45 (d, 1H), 7.17 (s, 1H), 6.81 (d, 1H), 4.62 (d, 2H) and 2.69 ppm (d, 3H); ES-MS m/z 513.3 [M+H]⁺, HPLC RT (min) 2.78.

### Example 21-p

### Preparation of 4-{[(2-{[(methylamino)carbonyl]amino}pyridin-4-yl)methyl]amino}-N-[4-(trifluoromethoxy)phenyl]-1,3-thiazole-5-carboxamide

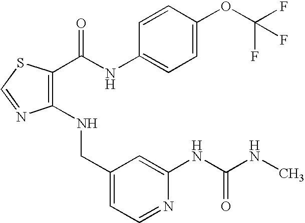

The title compound was prepared using the same method described for Example 21-o but starting with 4-amino-N-[4-(trifluoromethoxy)phenyl]-1,3-thiazole-5-carboxamide (Intermediate B-3) rather than Intermediate B-2 and proportional amounts of the other reaction components. The reaction mixture was heated at 60° C. in a foil wrapped flask for 24 h. The resulting final crude mixture was diluted with saturated aqueous sodium bicarbonate and extracted with ethyl acetate. The extract was washed 3 times with water and then with saturated brine. The extract was dried (Na₂SO₄) and evaporated in vacuo to yield a residue that was purified by chromatography on silica gel using a gradient from 0-100% ethyl acetate in hexane.

¹H NMR (300 MHz, DMSO-d₆) δ 9.75 (s, 1H), 9.23 (bs, 1H), 8.93 (s, 1H), 8.20 (bs, 1H), 8.05 (m, 2H), 7.78 (d, 2H), 7.30 (d, 2H), 7.15 (s, 1H), 6.83 (d, 1H), 4.62 (d, 2H) and 2.69 ppm (d, 3H); ES-MS m/z 467.4 [M+H]⁺, HPLC RT (min) 2.59.

### Example 21-q

### Preparation of 4-{[(2-{[(methylamino)carbonyl]amino}pyridin-4-yl)methyl]amino}-N-[3-(trifluoromethoxy)phenyl]-1,3-thiazole-5-carboxamide

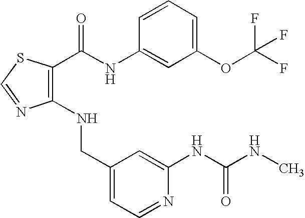

The title compound was prepared using the same method described for Example 21-o but starting with 4-amino-N-[3-(trifluoromethoxy)phenyl]-1,3-thiazole-5-carboxamide (Intermediate B-4) rather than Intermediate B-2 and proportional amounts of the other reaction components. The reaction mixture was heated at 60° C. in a foil wrapped flask for 24 h. The resulting final crude mixture was diluted with saturated aqueous sodium bicarbonate and extracted with ethyl acetate. The extract was washed 3 times with water and then with saturated brine. The extract was dried (Na₂SO₄) and evaporated in vacuo to yield a residue that was purified by chromatography on silica gel using a gradient from 0-100% ethyl acetate in hexane.

¹H NMR (300 MHz, DMSO-d₆) δ 9.82 (s, 1H), 9.19 (s, 1H), 8.93 (s, 1H), 8.22 (bs, 1H), 8.05 (m, 2H), 7.85 (s, 1H), 7.67 (d, 1H), 7.42 (t, 1H), 7.15 (s, 1H), 7.01 (d, 1H), 6.81 (d, 1H), 4.62 (d, 2H) and 2.69 ppm (d, 3H); ES-MS m/z 467.3 [M+H]⁺, HPLC RT (min) 2.61.

### Example 21-x

### Preparation of 4-{[(2-{[(methylamino)carbonyl]amino}pyridin-4-yl)methyl]amino}-N-{4-[(trifluoromethyl)thio]phenyl}-1,3-thiazole-5-carboxamide

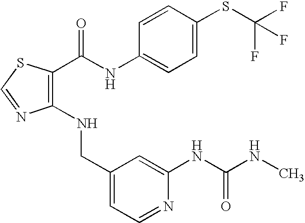

The title compound was prepared using the same method described for Example 21-o but starting with 4-amino-N-{4-[(trifluoromethyl)thio]phenyl}-1,3-thiazole-5-carboxamide (Intermediate B-5) rather than Intermediate B-2 and proportional amounts of the other reaction components. The reaction mixture was heated at 60° C. in a foil wrapped flask for 24 h. The resulting final crude mixture was diluted with saturated aqueous sodium bicarbonate and extracted with ethyl acetate. The extract was washed 3 times with water and then with saturated brine. The extract was dried (Na₂SO₄) and evaporated in vacuo to yield a residue that was purified by chromatography on silica gel using a gradient from 0-100% ethyl acetate in hexane.

¹H NMR (300 MHz, DMSO-d₆) δ 9.84 (s, 1H), 9.20 (s, 1H), 8.94 (s, 1H), 8.22 (bs, 1H), 8.07 (m, 2H), 7.86 (d, 2H), 7.63 (d, 2H), 7.15 (s, 1H), 6.81 (d, 1H), 4.62 (d, 2H) and 2.70 ppm (d, 3H); ES-MS m/z 483.4 [M+H]⁺, HPLC RT (min) 2.75.

### Example 21-y

### Preparation of 4-{[(2-{[(ethylamino)carbonyl]amino}pyridin-4-yl)methyl]amino}-N-[4-(trifluoromethoxy)phenyl]-1,3-thiazole-5-carboxamide

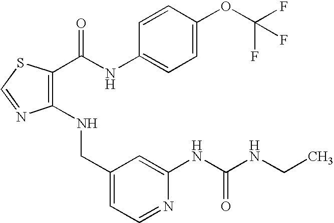

The title compound was prepared using the same method described for Example 21-o but starting with 4-amino-N-[4-(trifluoromethoxy)phenyl]-1,3-thiazole-5-carboxamide (Intermediate B-3) rather than Intermediate B-2 and proportionate amounts of Intermediate L rather than Intermediate N and also proportional amounts of the other reaction components. The reaction mixture was heated at 60° C. in a foil wrapped flask for 24 h. The resulting final crude mixture was diluted with saturated aqueous sodium bicarbonate and extracted with ethyl acetate. The extract was washed 3 times with water and then with saturated brine. The extract was dried (Na₂SO₄) and evaporated in vacuo to yield a residue that was purified by chromatography on silica gel using a gradient from 0-100% ethyl acetate in hexane.

¹H NMR (300 MHz, DMSO-d₆) δ 9.75 (s, 1H), 9.10 (s, 1H), 8.94 (s, 1H), 8.22 (bt, 1H), 8.05 (m, 2H), 7.79 (d, 2H), 7.31 (d, 2H), 7.21 (s, 1H), 6.81 (d, 1H), 4.62 (d, 2H), 3.15 (m, 2H) and 1.06 ppm (t, 3H); ES-MS m/z 481.1 [M+H]⁺, HPLC RT (min) 2.67.

### Example 21-z

### Preparation of 4-[({2-[(aminocarbonyl)amino]pyridin-4-yl}methyl)amino]-N-(2,2-difluoro-1,3-benzodioxol-5-yl)-1,3-thiazole-5-carboxamide

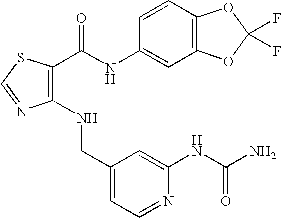

### Step 1: Preparation of 4-{[(2-{[(benzoylamino)carbonyl]amino}pyridin-4-yl)methyl]amino}-N-(2,2-difluoro-1,3-benzodioxol-5-yl)-1,3-thiazole-5-carboxamide

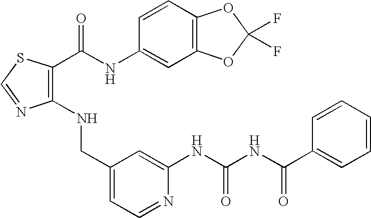

The title compound was prepared using the same method described for Example 1 but starting with proportionate amounts of N-({[4-(chloromethyl)pyridin-2-yl]amino}carbonyl)benzamide (Intermediate R) rather than Intermediate D and also proportional amounts of the other reaction components. The reaction mixture was heated at 60° C. in a foil wrapped flask for 24 h. The resulting final crude mixture was diluted with saturated aqueous sodium bicarbonate and extracted with ethyl acetate. The extract was washed 3 times with water and then with saturated brine. The extract was dried (Na₂SO₄) and evaporated in vacuo to yield a residue that was purified by chromatography on silica gel using a gradient from 0-100% ethyl acetate in hexane.

ES-MS m/z 553.1 [M+H]⁺ and 575.0 [M+Na]⁺, HPLC RT (min) 3.44.

### Step 2: Preparation of 4-[(2-[(aminocarbonyl)amino]pyridin-4-yl I meth I)amino]-N-(2,2-difluoro-1,3-benzodioxol-5-yl)-1,3-thiazole-5-carboxamide

A suspension of 4-{[(2-{[(benzoylamino)carbonyl]amino}pyridin-4-yl)methyl]amino}-N-(2,2-difluoro-1,3-benzodioxol-5-yl)-1,3-thiazole-5-carboxamide (36 mg, 0.07 mmol) and potassium carbonate (9.0 mg, 0.07 mmol) in absolute ethanol (0.95 ml) was stirred at 85° C. in a sealed tube for 1.5 hr. The resultant mixture was evaporated and the residue was chromatographed on silica gel using a gradient from 1-10% methanol in dichloromethane to yield pure title compound.

¹H NMR (300 MHz, DMSO-d₆) δ 9.77 (s, 1H), 9.07 (s, 1H), 8.94 (s, 1H), 8.05 (m, 2H), 7.80 (s, 1H), 7.37 (m, 2H), 7.27 (s, 1H), 7.05 (very broad s, 2H), 6.80 (d, 1H), and 4.62 ppm (d, 2H); ES-MS m/z 449.3 [M+H]⁺, HPLC RT (min) 2.47.

### Examples 21-b TO 21-x

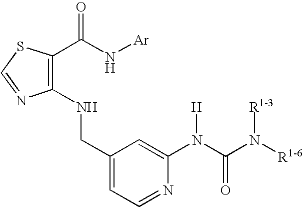

### Step 1: Preparation of various N-[4-(chloromethyl)pyridin-2-yl]ureas with other N′-substituents

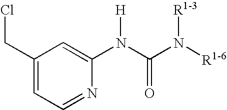

By using the methods described for preparation of Intermediate L and by substituting the appropriate alkyl or aryl isocyanate rather than ethylisocyanate in either DMF or dichloromethane as solvent, the intermediates with the above structure in which the R¹⁻³ and R¹⁻⁶ groups are as found in examples 21-b to 21-g, 21-i, and 21-o to 21-w of Table A can be prepared. By using the methods described for the preparation of Intermediate E, step 2 but using the appropriate carbamoyl chloride rather than acetoxyacetyl chloride, the intermediates which lead to Examples 21-k to 21-n of Table A can be prepared. In all cases the appropriate isocyanate or carbamoyl chloride is either commercially available or the synthesis is straightforward to one skilled in the art and is reported in the general literature.

### Step 2: Preparation of the Title Compounds

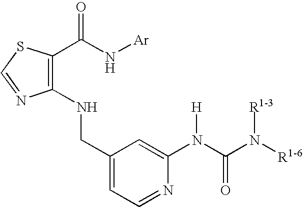

The title compounds can be prepared using the same method described for Example 1 but starting with either 4-amino-N-(2,2-difluoro-1,3-benzodioxol-5-yl)-1,3-thiazole-5-carboxamide (Intermediate A) or another material from the list of Intermediates B, B-2, B-3, B-4 or B-5 instead of Intermediate A and proportionate amounts of the appropriate Intermediate from Step 1 above rather than Intermediate D and also proportional amounts of the other reaction components. The reaction mixture is heated at 60° C. in a foil wrapped flask for between 2 and 24 h until an LCMS analysis of the reaction mixture shows substantial conversion to products. The resulting final crude mixture is diluted with saturated aqueous sodium bicarbonate and extracted 3 times with ethyl acetate. The combined extracts are dried (Na₂SO₄) and evaporated in vacuo to yield a residue that is purified by preparative C18 HPLC using water to acetonitrile gradient (usually 10-50%) with added 0.05-0.1% TFA. The free base is prepared from the TFA salt by addition of saturated aqueous NaHCO₃ to the fractions containing the product and extraction with dichloromethane followed by drying of the extract (Na₂SO₄) and concentration in vacuo to yield pure title compound. In the case of Title compounds 21-h and 21-j, the initial blocked products 21-g and 21-i respectively are converted to the final Title compounds by treatment with potassium carbonate in methanol or ethanol. Structures and names of the Title Compounds 21-a to 21-x are shown in Table A.

TABLE A

| Example | | |
|---|---|---|
| Number | Structure | Name |
| 21-a | 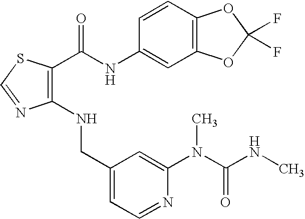 | N-(2,2-difluoro-1,3-benzodioxol-5-yl)-4-{[(2-{methyl[(methylamino)carbonyl]amino}pyridin-4-yl)methyl]amino}-1,3-thiazole-5-carboxamide |
| 21-b | 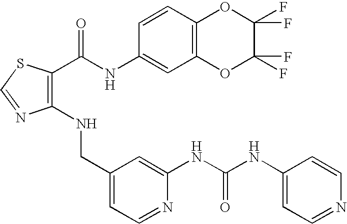 | 4-{[(2-{[(pyridin-4-ylamino)carbonyl]amino}pyridin-4-yl)methyl]amino}-N-(2,2,3,3-tetrafluoro-2,3-dihydro-1,4-benzodioxin-6-yl)-1,3-thiazole-5-carboxamide |
| 21-c | 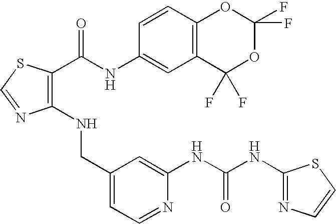 | N-(2,2,4,4-tetrafluoro-4H-1,3-benzodioxin-6-yl)-4-{[(2-{[(1,3-thiazol-2-ylamino)carbonyl]amino}pyridin-4-yl)methyl]amino}-1,3-thiazole-5-carboxamide |
| 21-d | 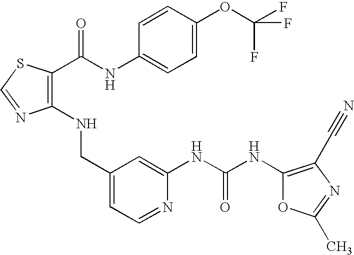 | 4-({[2-({[(4-cyano-2-methyl-1,3-oxazol-5-yl)amino]carbonyl}amino)pyridin-4-yl]methyl}amino)-N-[4-(trifluoromethoxy)phenyl]-1,3-thiazole-5-carboxamide |
| 21-e | 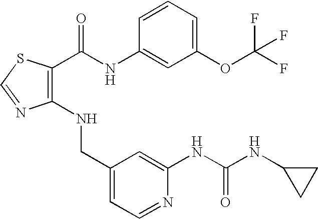 | 4-{[(2-{[(cyclopropylamino)carbonyl]amino}pyridin-4-yl)methyl]amino}-N-[3-(trifluoromethoxy)phenyl]-1,3-thiazole-5-carboxamide |
| 21-f | 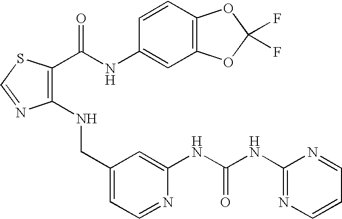 | N-(2,2-difluoro-1,3-benzodioxol-5-yl)-4-{[(2-{[(pyrimidin-2-ylamino)carbonyl]amino}pyridin-4-yl)methyl]amino}-1,3-thiazole-5-carboxamide |
| 21-g | 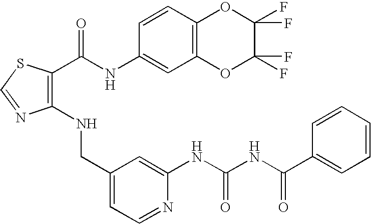 | 4-{[(2-{[(benzoylamino)carbonyl]amino}pyridin-4-yl)methyl]amino}-N-(2,2,3,3-tetrafluoro-2,3-dihydro-1,4-benzodioxin-6-yl)-1,3-thiazole-5-carboxamide |
| 21-h | 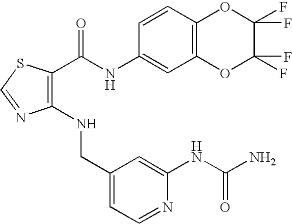 | 4-[({2-[(aminocarbonyl)amino]pyridin-4-yl}methyl)amino]-N-(2,2,3,3-tetrafluoro-2,3-dihydro-1,4-benzodioxin-6-yl)-1,3-thiazole-5-carboxamide |
| 21-i | 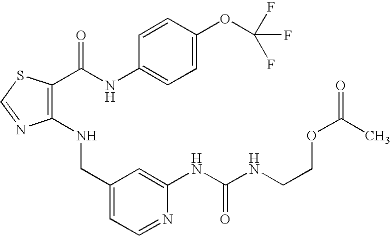 | 2-[({[4-({[5-({[4-(trifluoromethoxy)phenyl]amino}carbonyl)-1,3-thiazol-4-yl]amino}methyl)pyridin-2-yl]amino}carbonyl)amino]ethyl acetate |
| 21-j | 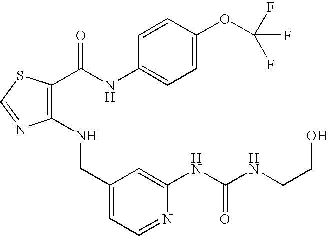 | 4-({[2-({[(2-hydroxyethyl)amino]carbonyl}amino)pyridin-4-yl]methyl}amino)-N-[4-(trifluoromethoxy)phenyl]-1,3-thiazole-5-carboxamide |
| 21-k | 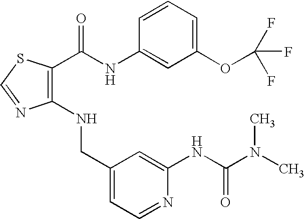 | 4-{[(2-{[(dimethylamino)carbonyl]amino}pyridin-4-yl)methyl]amino}-N-[3-(trifluoromethoxy)phenyl]-1,3-thiazole-5-carboxamide |
| 21-l | 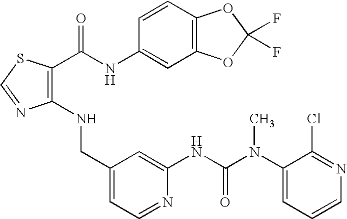 | 4-({[2-({[(2-chloropyridin-3-yl)(methyl)amino]carbonyl}amino)pyridin-4-yl]methyl}amino)-N-(2,2-difluoro-1,3-benzodioxol-5-yl)-1,3-thiazole-5-carboxamide |
| 21-m | 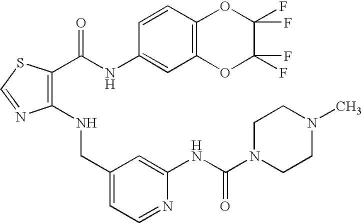 | 4-methyl-N-(4-{[(5-{[(2,2,3,3-tetrafluoro-2,3-dihydro-1,4-benzodioxin-6-yl)amino]carbonyl}-1,3-thiazol-4-yl)amino]methyl}pyridin-2-yl)piperazine-1-carboxamide |
| 21-n | 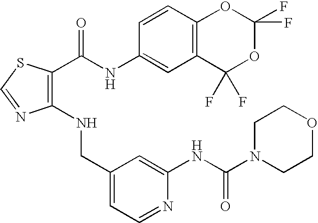 | N-(4-{[(5-{[(2,2,4,4-tetrafluoro-4H-1,3-benzodioxin-6-yl)amino]carbonyl}-1,3-thiazol-4-yl)amino]methyl}pyridin-2-yl)morpholine-4-carboxamide |
| 21-o | 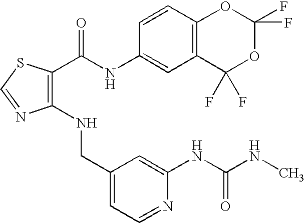 | 4-{[(2-{[(methylamino)carbonyl]amino}pyridin-4-yl)methyl]amino}-N-(2,2,4,4-tetrafluoro-4H-1,3-benzodioxin-6-yl)-1,3-thiazole-5-carboxamide |
| 21-p | 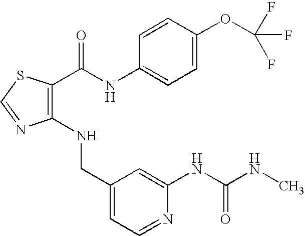 | 4-{[(2-{[(methylamino)carbonyl]amino}pyridin-4-yl)methyl]amino}-N-[4-(trifluoromethoxy)phenyl]-1,3-thiazole-5-carboxamide |
| 21-q | 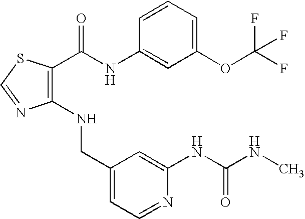 | 4-{[(2-{[(methylamino)carbonyl]amino}pyridin-4-yl)methyl]amino}-N-[3-(trifluoromethoxy)phenyl]-1,3-thiazole-5-carboxamide |
| 21-r | 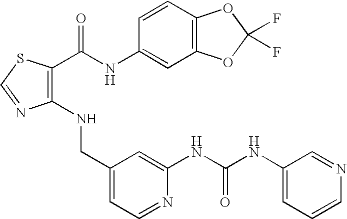 | N-(2,2-difluoro-1,3-benzodioxol-5-yl)-4-{[(2-{[(pyridin-3-ylamino)carbonyl]amino}pyridin-4-yl)methyl]amino}-1,3-thiazole-5-carboxamide |
| 21-s | 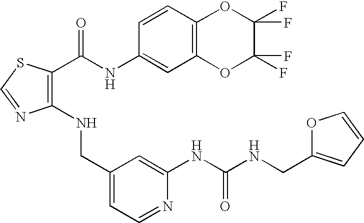 | 4-({[2-({[(2-furylmethyl)amino]carbonyl}amino)pyridin-4-yl]methyl}amino)-N-(2,2,3,3-tetrafluoro-2,3-dihydro-1,4-benzodioxin-6-yl)-1,3-thiazole-5-carboxamide |
| 21-t | 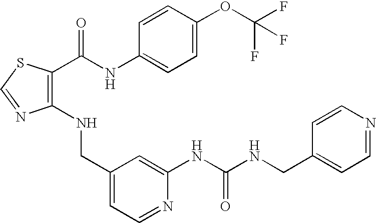 | 4-({[2-({[(pyridin-4-ylmethyl)amino]carbonyl}amino)pyridin-4-yl]methyl}amino)-N-[4-(trifluoromethoxy)phenyl]-1,3-thiazole-5-carboxamide |
| 21-u | 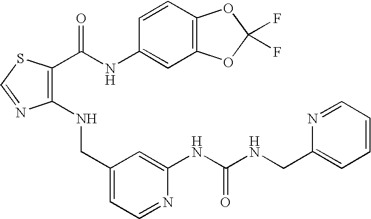 | N-(2,2-difluoro-1,3-benzodioxol-5-yl)-4-({[2-({[(pyridin-2-ylmethyl)amino]carbonyl}amino)pyridin-4-yl]methyl}amino)-1,3-thiazole-5-carboxamide |
| 21-v | 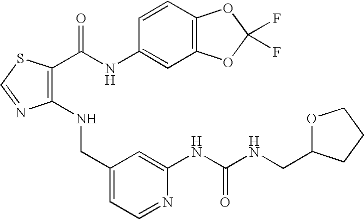 | N-(2,2-difluoro-1,3-benzodioxol-5-yl)-4-({[2-({[(tetrahydrofuran-2-ylmethyl)amino]carbonyl}amino)pyridin-4-yl]methyl}amino)-1,3-thiazole-5-carboxamide |
| 21-w | 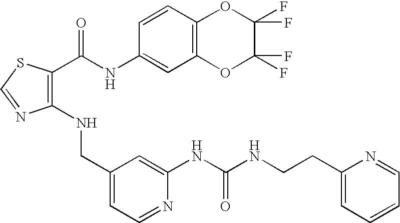 | 4-({[2-({[(2-pyridin-2-ylethyl)amino[carbonyl}amino)pyridin-4-yl]methyl}amino)-N-(2,2,3,3-tetrafluoro-2,3-dihydro-1,4-benzodioxin-6-yl)-1,3-thiazole-5-carboxamide |
| 21-x | 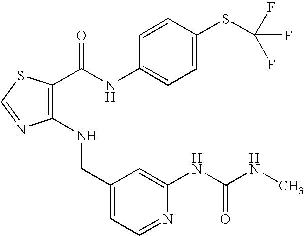 | 4-{[(2-{[(methylamino)carbonyl]amino}pyridin-4-yl)methyl]amino}-N-{4-[(trifluoromethyl)thio]phenyl}-1,3-thiazole-5-carboxamide |

### Example 22

### Preparation of 4-{([2-[(anilinocarbonyl)amino]pyridin-4-yl}methyl)amino]-N-(2,2,3,3-tetrafluoro-2,3-dihydro-1,4-benzodioxin-6-yl)-1,3-thiazole-5-carboxamide

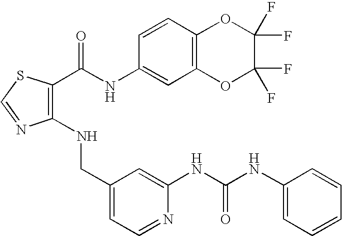

The title compound is prepared using the same method described for Example 1 but starting with 4-amino-N-(2,2,3,3-tetrafluoro-2,3-dihydro-1,4-benzodioxin-6-yl)-1,3-thiazole-5-carboxamide rather than 4-amino-N-(2,2-difluoro-1,3-benzodioxol-5-yl)-1,3-thiazole-5-carboxamide (Intermediate B) and proportionate amounts of Intermediate M rather than Intermediate D and also proportional amounts of the other reaction components. The reaction mixture is heated at 60° C. in a foil wrapped flask for between 2 and 24 h until an LCMS analysis of the reaction mixture shows substantial conversion to products. The resulting final crude mixture is diluted with saturated aqueous sodium bicarbonate and extracted 3 times with ethyl acetate. The combined extracts are dried (Na₂SO₄) and evaporated in vacuo to yield a residue that is purified by preparative C18 HPLC using water to acetonitrile gradient (usually 10-50%) with added 0.05-0.1% TFA. The free base is prepared from the TFA salt by addition of saturated aqueous NaHCO₃ to the fractions containing the product and extraction with dichloromethane followed by drying of the extract (Na₂SO₄) and concentration in vacuo to yield pure title compound.

### Example 23

### Preparation of N-(2,2-difluoro-1,3-benzodioxol-5-yl)-4-({[2-({[(3-methoxyphenyl)amino]carbonyl}amino)pyridin-4-yl]methyl}amino)-1,3-thiazole-5-carboxamide

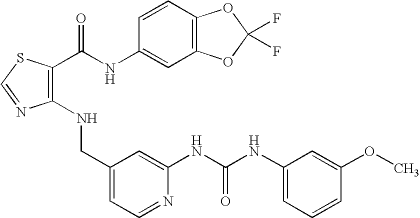

A mixture of 4-{[(2-aminopyridin-4-yl)methyl]amino}-N-(2,2-difluoro-1,3-benzodioxol-5-yl)-1,3-thiazole-5-carboxamide (Example 15, 0.18 mmol), 1-isocyanato-3-methoxybenzene (0.18 mmol) and N-ethyl-N-isopropylpropan-2-amine (2 drops) in dichloromethane (1.5 mL) is stirred for 16 h under nitrogen. The product may crystallize out of solution, in which case it is collected by filtration, washed with dichloromethane, methanol and diethyl ether and dried in vacuo to yield pure title compound.

As a general procedure for the reaction of 4-{[(2-aminopyridin-4-yl)methyl]amino}-N-(2,2-difluoro-1,3-benzodioxol-5-yl)-1,3-thiazole-5-carboxamide (Example 15) with other isocyanates, if the product does not crystallize as a pure material, it is evaporated and either purified by chromatography on silica gel using a gradient from 0 to 100% ethyl acetate in hexane or by preparative C18 HPLC using a water to acetonitrile gradient (usually 10-50%) with added 0.05-0.1% TFA. Free base is prepared from the TFA salt by addition of saturated aqueous NaHCO₃ to the fractions containing the product and extraction with dichloromethane followed by drying of the extract (Na₂SO₄) and evaporated in vacuo to yield pure title compound.

### Examples 24

### Preparation of N-(2,2-difluoro-1,3-benzodioxol-5-yl)-4-({[2-(methylamino)pyrimidin-4-yl]methyl}amino)-1,3-thiazole-5-carboxamide

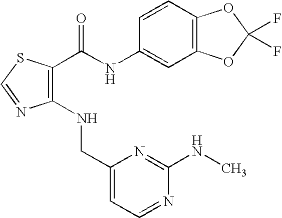

### Step 1. Preparation of 4-{[(2,6-dichloropyrimidin-4-yl)methyl]amino}-N-(2,2-difluoro-1,3-benzodioxol-5-yl)-1,3-thiazole-5-carboxamide

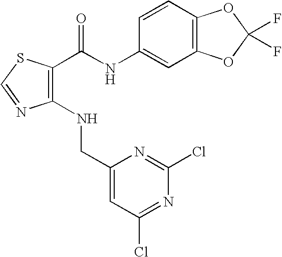

A mixture of 2,4-dichloro-6-(chloromethyl)pyrimidine (Intermediate 0, 99 mg, 0.5 mmol) and sodium iodide (75 mg, 0.5 mmol) in anhydrous DMF (0.5 mL) was stirred under nitrogen until a solution was formed and then 4-amino-N-(2,2-difluoro-1,3-benzodioxol-5-yl)-1,3-thiazole-5-carboxamide (Intermediate A, 100 mg, 0.33 mmol) was added. The resulting mixture was heated and stirred under nitrogen at 40° C. for 4 h and then at 59° C. overnight. After TLC analysis had shown some remaining thiazole starting material, additional 2,4-dichloro-6-(chloromethyl)pyrimidine (30 mg) was added and the reaction mixture again was heated at 59° C. for another 2 h. The resulting final crude mixture was diluted with saturated aqueous sodium bicarbonate and extracted 3 times with ethyl acetate. The combined extracts were dried (Na₂SO₄) and evaporated in vacuo to yield a residue that was purified by chromatography on silica gel using a gradient from 10-40% ethyl acetate in hexane. The yield of pure material was 15 mg (10%).

¹H NMR (300 MHz, CD₂Cl₂) δ 8.60 (s, 1H), 7.97 (bt, 1H), 7.67 (d, 1H, meta coupling), 7.35 (s, 1H), 7.18 (bs, 1H), 7.07 (s, 2H) and 4.84 ppm (d, 2H); ES-MS m/z 459.9 [M+H]⁺, HPLC RT (min) 3.78.

### Step 2. Preparation of 4-({[6-chloro-2-(methylamino)pyrimidin-4-yl]methyl}amino)-N-(2,2-difluoro-1,3-benzodioxol-5-yl)-1,3-thiazole-5-carboxamide and 4-({[2-chloro-6-(methylamino)pyrimidin-4-yl]methyl}amino)-N-(2,2-difluoro-1,3-benzodioxol-5-yl)-1,3-thiazole-5-carboxamide

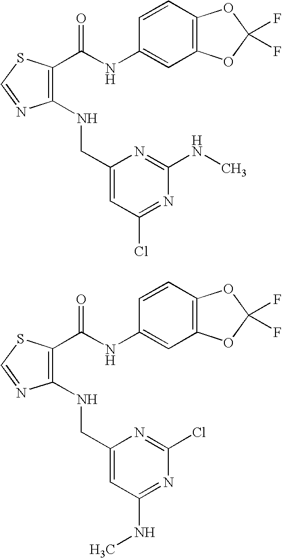

A solution of 4-{[(2,6-dichloropyrimidin-4-yl)methyl]amino}-N-(2,2-difluoro-1,3-benzodioxol-5-yl)-1,3-thiazole-5-carboxamide (the product of step 1, 0.2 mmol) and methylamine (0.4 mmol) in methanol (1.2 mL) is stirred in a sealed tube until HPLC indicates a substantial conversion to products. The products are purified by direct injection on a preparative HPLC to yield purified isomers wherein the 2-(methylamino)pyrimidin-4-yl isomer is expected to be the major product.

### Step 3. Preparation of the Title Compound

A mixture of 4-({[6-chloro-2-(methylamino)pyrimidin-4-yl]methyl}amino)-N-(2,2-difluoro-1,3-benzodioxol-5-yl)-1,3-thiazole-5-carboxamide (the minor product of step 2, 0.18 mmol) plus palladium (II) hydroxide (0.36 mmol) and ammonium formate (1.76 mmol) in ethyl acetate (15 mL) and methanol (15 mL) is stirred with heating at reflux for 16 h. The product solution is filtered using Celite® filter aid and evaporated in vacuo and the residue is purified by preparative C18 HPLC using a gradient from 5 to 45% acetonitrile in water plus 0.1% TFA. Evaporation of product containing fractions yields pure compounds as TFA salts. Alternatively, the fractions containing the product are mixed with saturated aqueous NaHCO₃ and extracted with dichloromethane. The extracts are dried (Na₂SO₄) and evaporated in vacuo to yield pure free base title compound.

### Examples 25

### Preparation of N-(2,2-difluoro-1,3-benzodioxol-5-yl)-4-{[(2-{[3-(4-methylpiperazin-1-yl)propyl]amino}pyrimidin-4-yl)methyl]amino}-1,3-thiazole-5-carboxamide

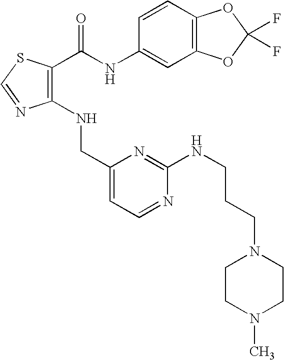

### Step 1. Preparation of 4-{[(6-chloro-2-{[3-(4-methylpiperazin-1-yl)propyl]amino}pyrimidin-4-yl)methyl]amino}-N-(2,2-difluoro-1,3-benzodioxol-5-yl)-1,3-thiazole-5-carboxamide and 4-{[(2-chloro-6-{[3-(4-methylpiperazin-1-yl)propyl]amino}pyrimidin-4-yl)methyl]amino}-N-(2,2-difluoro-1,3-benzodioxol-5-yl)-1,3-thiazole-5-carboxamide

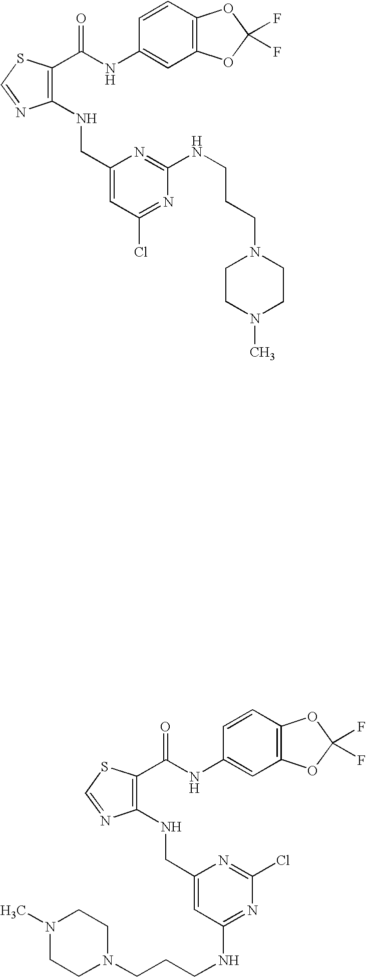

A solution of 4-{[(2,6-dichloropyrimidin-4-yl)methyl]amino}-N-(2,2-difluoro-1,3-benzodioxol-5-yl)-1,3-thiazole-5-carboxamide (the product of step 1, Example 24, 0.09 mmol), 3-(4-methylpiperazin-1-yl)propan-1-amine (0.26 mmol) and 2 drops aqueous HCl (1 N) in isopropanol (3 mL) is stirred in a sealed tube for 16 h or until LCMS indicates substantial conversion to products. The product mixture is filtered, evaporated and purified by preparative C18 HPLC to yield pure separated isomers in which the first compound above is expected to be the minor product.

### Step 2. Preparation of the Title Compound

A mixture of 4-{[(6-chloro-2-{[3-(4-methylpiperazin-1-yl)propyl]amino}pyrimidin-4-yl)methyl]amino}-N-(2,2-difluoro-1,3-benzodioxol-5-yl)-1,3-thiazole-5-carboxamide (0.1 mmol) plus palladium (II) hydroxide (0.01 mmol) and ammonium formate (0.71 mmol) in ethanol (2.5 mL) is reacted in a sealed microwave reactor tube at 150° C. for 0.5 h. The product solution is filtered using Celite® filter aid and evaporated in vacuo and the residue is purified by preparative C18 HPLC using a gradient from 10 to 50% acetonitrile in water plus 0.1% TFA. Evaporation of product containing fractions yields pure compounds as TFA salts. Alternatively, the fractions containing the product are mixed with saturated aqueous NaHCO₄ and extracted with dichloromethane. The extracts are dried (Na₂SO₄) and evaporated in vacuo to yield pure free base title compound.

### Example 26

### Preparation of 4-{[(2-chloropyridin-4-yl)methyl]amino}-N-(2,2-difluoro-1,3-benzodioxol-5-yl)-1,3-thiazole-5-carboxamide

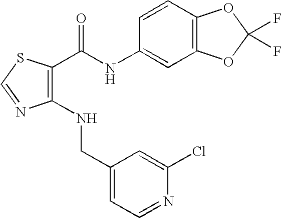

The title compound is prepared using the same method described for Example 1 but starting with 4-amino-N-(2,2-difluoro-1,3-benzodioxol-5-yl)-1,3-thiazole-5-carboxamide and proportionate amounts of Intermediate P rather than Intermediate D.

### Example 27

### Preparation of N-(2,2-difluoro-1,3-benzodioxol-5-yl)-4-({[2-(methylamino)pyridin-4-yl]methyl}amino)-1,3-thiazole-5-carboxamide

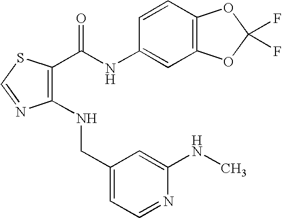

A solution of 4-{[(2-chloropyridin-4-yl)methyl]amino}-N-(2,2-difluoro-1,3-benzodioxol-5-yl)-1,3-thiazole-5-carboxamide (Example 26, 0.48 mmol) and methylamine hydrochloride (4.79 mmol) in pyridine (3 mL) is heated at 200° C. in a sealed tube for 16 h. The resulting solution is diluted with water and extracted with ethyl acetate. The combined extracts are washed with saturated NaHCO₃ and then brine. The extracts are dried (Na₂SO₄) and evaporated in vacuo to yield a residue that is purified by preparative C18 HPLC using water to acetonitrile gradient plus 0.1% TFA to yield pure title compound after conversion to free base as described in the preparation of Example 25.

### Example 28

### Preparation of N-(2,2-difluoro-1,3-benzodioxol-5-yl)-4-[{(2-[(2-hydroxyethyl)amino]pyridin-4-yl]methyl)amino}-1,3-thiazole-5-carboxamide

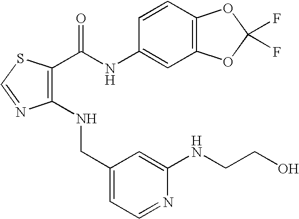

A solution of 4-{[(2-chloropyridin-4-yl)methyl]amino}-N-(2,2-difluoro-1,3-benzodioxol-5-yl)-1,3-thiazole-5-carboxamide (Example 26, 0.48 mmol) and 2-aminoethanol (4.79 mmol) in pyridine (3 mL) is heated at 200° C. in a sealed tube for 16 h. The resulting solution is diluted with water and extracted with ethyl acetate. The combined extracts are washed with saturated NaHCO₃ and then brine. The extracts are dried (Na₂SO₄) and evaporated in vacuo to yield a residue that is purified by preparative C18 HPLC using water to acetonitrile gradient plus 0.1% TFA to yield pure title compound after conversion to free base as described in the preparation of Example 25.

### Example 29

### Preparation of N-(2,2-difluoro-1,3-benzodioxol-5-yl)-4-[{(2-{[2-(2-hydroxyethoxy)ethyl]amino}pyridyl-4-yl)methyl]amino}-1,3-thiazole-5-carboxamide

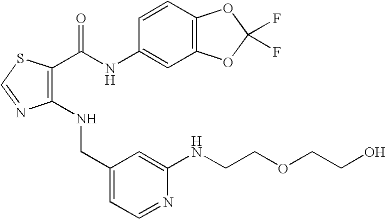

A solution of 4-{[(2-chloropyridin-4-yl)methyl]amino}-N-(2,2-difluoro-1,3-benzodioxol-5-yl)-1,3-thiazole-5-carboxamide (Example 26, 0.48 mmol) and 2-(2-aminoethoxy)ethanol (4.79 mmol) in pyridine (3 mL) is heated at 200° C. in a sealed tube for 16 h. The resulting solution is diluted with water and extracted with ethyl acetate. The combined extracts are washed with saturated NaHCO₃ and then brine. The extracts are dried (Na₂SO₄) and evaporated in vacuo to yield a residue that is purified by preparative C18 HPLC using water to acetonitrile gradient plus 0.1% TFA to yield pure title compound after conversion to free base as described in the preparation of Example 25.

### Example 30

### Preparation of N-(2,2-difluoro-1,3-benzodioxol-5-yl)-4-[({2-[(4-methyl-1,3-thiazol-2-yl)amino]pyridin-4-yl]methyl)amino}-1,3-thiazole-5-carboxamide

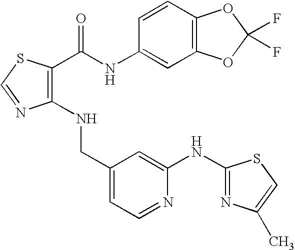

The title compound is prepared using the same method described for Example 1 but starting with 4-amino-N-(2,2-difluoro-1,3-benzodioxol-5-yl)-1,3-thiazole-5-carboxamide (Intermediate A) and proportionate amounts of 4-(chloromethyl)-N-(4-methyl-1,3-thiazol-2-yl)pyridin-2-amine (Intermediate Q) rather than Intermediate D.

### B. EVALUATION OF PHYSIOLOGICAL ACTIVITY

The utility of the compounds of the present invention can be illustrated, for example, by their activity in the P-AKT/PKB Cytoblot Assay described below. The involvement of the P-AKT/PKB[PI3K/AKt] pathway as a target for cancer chemotherapy has been recognized in the art. For example, see F. Chang et al, Involvement of PI3K/Akt pathway in cell cycle progression, apoptosis, and neoplastic transformation: a target for cancer chemotherapy, Leukemia, 2003, 17: p. 590-603; K. A. West et al, Activation of the PI3K/Akt pathway and chemotherapeutic resistance, Drug Resistance Updates, 2002, 5: p. 234-248; and P. Sen et al, Involvement of the Akt/PKB signaling pathway with disease processes, Molecular and Cellular Biochemistry, 2003, 253: p. 241-246.

P-AKT/PKB Cytoblot Assay Protocol with H209 Cells

H209 small cell lung carcinoma cells in log phase were plated at 50,000 cells/well in 96-well poly-lysine coated, clear bottom/black-sided plates (Becton-Dickinson, USA Cat # 354640) in 100 Ill RPMI medium containing 0.1% (w/v) BSA, and incubated overnight at 37° C. in 5% CO₂ incubator. The following day, compounds (10 mM stock solutions in DMSO) were added to the plates to generate final concentrations of 0.0, 0.01, 0.03, 0.1, 0.3, 1.0, 3.0 and 10 μM for IC₅₀ determinations and incubated for 1 hour at 37° C. Cells were then left untreated or stimulated with Stem Cell Factor (SCF: Biosource Cat # PHC2116) at a final concentration of 25 ng/mL for 5 minutes at 37° C. in 5% CO₂ incubator. The media was then removed using a vacuum manifold and the cells were washed once with Tris Buffered Saline (TBS). Cells were then fixed by adding 200 μl of cold 3.7% (v/v) formaldehyde in TBS to each well for 15 minutes at 4° C. After removal of the formaldehyde, the cells were treated with the addition of 50 μl of methanol (at −20° C.) to each well for 5 minutes. After removal of the methanol, 200 μl of 1% (w/v) BSA in TBS was added to each well to block non-specific antibody binding sites and the plate was incubated at room temperature for 30 minutes.

After removal of the blocking buffer, 50 μl of p-(S473) AKT rabbit polyclonal antibody (Cell Signaling, USA Cat # 9277S) was added at a dilution of 1:250 in 0.1% (w/v) BSA in TBS, and the plate was incubated at room temperature for 1 hour. Plates were then washed 3 times with cold TBS containing 0.05% (v/v) Tween 20 (TBS-T) and 100 μl of Horseradish peroxidase (HRP)-conjugated goat-anti-rabbit antibody (Amersham, USA Cat # NA934V) at a dilution of 1:250 in TBS-T was added and the plate was incubated at room temperature for 1 h. After washing with ice-cold TBS-T four times, 100 μl of Enhanced Chemiluminescence (ECL) reagent (Amersham, USA Cat# RPN2209) was added to each well and mixed on a mini-orbital shaker for 1 min. The plate was then read on a Perkin Elmer Victor 5 Multilabel Counter (#1420-0421).

Compounds of examples 1, 2, 3, 6, 7, 8, 9, 10, 11, 12, 13, 14, 15, 16, 17, 18, 20 and 21 were tested in the above P-AKT/PKB Cytoblot assay, with the result that these examples exhibited IC₅₀ values of less than 500 nM. In one embodiment, the present invention relates to a compound which exhibits an IC₅₀ value of less than 500 nM in this assay.
The utility of the compounds of the present invention can also be illustrated, for example, by their activity in the phosph-ERK Assay described below.
Growth-factor induction of the RAS/MEK/ERK signaling pathway leads to the induction of phosphorylation of a number of proteins including phospho-ERK (See C. J. Marshall, MAP kinase kinase kinase, MAP kinase kinase and MAP kinase, Current Opinions in Genetic Development, 1994, 4: p. 82-89). The importance of this pathway in cancer biology has been recognized in the art. Activation of the RAS signaling pathway is an important mechanism by which cancer develops (R. Herrera, et al, Unraveling the complexities of the Raf/MAP kinase pathway for pharmacological intervention, Trends Mol. Med., 2002, 8: p. S27-31). Mutational activation of RAS or downstream effectors as well as growth factor induction of this pathway leads to increased tumor cell proliferation and survival (A. A. Adjei, Blocking oncogenic RAS signaling for cancer therapy, J. Natl. Cancer Inst., 2001, 93(14): p. 1062-1074; J Schlessinger, Cell signaling by receptor tyrosine kinases, Cell, 2000, 103: p. 211-225).
Phospho-ERK Cytoblot Assay Protocol with MDA-MB 231 Cells
MDA-MB-231 cells in log phase were plated at 25,000 cells/well in 96-well opaque plates (Falcon, USA Cat # 353296) in 100 μL RPMI medium containing 10% (w/v) FBS, and incubated overnight at 37° C. in 5% CO₂ incubator. The following day, the growth medium was removed from the plate by aspiration and replaced with RPMI medium containing 0.1% BSA and example compounds diluted to generate final concentrations of 0.0, 0.001, 0.003, 0.01, 0.03, 0.1, 0.3, 1 and 3 μM. Cells were incubated with compound for 1 hour at 37° C. in a 5% CO₂ incubator. The media was then removed from the plate by aspiration and the cells were washed once with 180 μL/well cold Tris Buffered Saline (TBS). After removal of the wash buffer, the cells were fixed by adding 180 μL of cold 3.7% (v/v) formaldehyde in TBS to each well for 1 hour at 4° C. After removal of the formaldehyde, the cells were treated with the addition of 60 μL of −20° C. methanol to each well for 5 minutes at 4° C. The methanol was removed and the cells were washed with 180 μL/well of 5% (w/v) BSA in TBS. To block non-specific antibody binding sites, each well was treated with 180 μL/well 5% BSA (w/v) in TBS for thirty minutes at room temperature. After removal of the blocking buffer, 50 μL of an anti-phospho-p44/42 MAP kinase (Thr202/Tyr204) rabbit polyclonal antibody (Cell Signaling, USA Cat # 9101) was added to each well at a dilution of 1:1000 in 5% (w/v) BSA in TBS, and the plate was incubated at 4° C. overnight. Plates were then washed three times with 300 μL/well TBS at room temperature. The plates were then incubated with 50 μL of Horseradish peroxidase (HRP)-conjugated goat-anti-rabbit antibody (Amersham, USA Cat. # NA934V) at a dilution of 1:1000 in 5% BSA-TBS at room temperature for 1 hr. After washing the plate three times with 300 μL/well TBS, 60 μL of Enhanced Chemiluminescence (ECL) reagent (Amersham, USA Cat# RPN2209) was added to each well and incubated at room temperature for five minutes. The plate was then read on a Perkin Elmer Victor 5 Multilabel Counter (#1420-0421).
The compounds of examples 1, 2, 7, 8, 9, 10, 11, 13, 14, 17, 18, 20 and 21 were tested and showed an IC₅₀ value of less than 3 μM in this assay. In one embodiment, the present invention relates to a compound which exhibits an IC₅₀ value of less than 3 μM.
The utility of the compounds of the present invention can also be illustrated, for example, by their activity in the flk-1 (murine VEGFR2) Assay described below.
The VEGF-VEGFR2 signaling pathway has been extensively characterized as an important regulator of angiogenesis and tumor angiogeneisis (See G. Yancopoulos et al, Vascular-specific growth factors and blood vessel formation, Nature, 2000, 407: p. 242-248; D. Shweiki et al, Induction of vascular endothelial growth factor expression by hypoxia and by glucose deficiency in multi cell spheroids: Implications for tumor angiogenesis, Proc. Natl. Acad. Sci, 1995, 92: p. 768-772). Inhibition of tumor cell growth by blocking this pathway has been well documented in the art. Administration of soluble VEGFR2 receptors inhibits the growth of a wide variety of tumors (See C. Bruns et al, Vascular endothelial growth factor is an in vivo survival factor for tumor endothelium in a murine model of colorectal liver metastases, Cancer, 2000, 89: p. 495-499; B. Millauer et al, Glioblastoma growth inhibited in vivo by a dominant-negative FLK-1 mutant, Nature, 1994, 367: p. 576-579). Neutralizing antibodies to VEGF or VEGFR2 and VEGF antisense suppress tumor growth in vivo (See K. Kim et al, Inhibition of vascular endothelial growth factor-induced angiogenesis suppresses tumor growth in vivo, Nature, 1993, 362: p. 841-844; M. Prewett et al, Antivascular endothelial growth factor receptor (fetal liver kinase 1) monoclonal antibody inhibits tumor angiogenesis and growth of several mouse and human tumors, Cancer Research, 1999, 59: p. 5209-5218; M. Saleh et al, Inhibition of growth of C6 glioma cells in vivo by expression of antisense vascular endothelial growth factor sequence, Cancer Research, 1996, 56: p. 393-401).
Flk-1 (murine VEGFR-2) Biochemical Assay
This assay was performed in 96-well opaque plates (Costar, USA Cat #3915) in the TR-FRET format. Reaction conditions were as follows: 10 μM ATP, 25 nM poly (Glu,Tyr)-biotin (CIS BIO International, USA Cat#61GT0BLD), 2 nM Eu-labelled phospho-Tyr Ab (Perkin Elmer, USA Cat#AD0067), 10 nM Strepavidin-APC (Perkin Elmer, USA Cat#CR130-100), 7 nM Flk-1 (kinase domain), 1% DMSO, 50 mM HEPES pH 7.5, 10 mM MgCl₂, 0.1 mM EDTA, 0.015% BRIJ, 0.1 mg/mL BSA, 0.1% mercapto-ethanol. Prior to the addition of enzyme, compounds were added to final concentrations ranging from 10 μM to 4.56 nM in 1% DMSO. The reaction was initiated upon addition of enzyme. Final reaction volume in each well was 100 μL. Time-resolved fluorescence was read after excitation at 340 nM. Emission readings were taken at both 665 and 615 nM on a Perkin Elmer Victor V Multilabel counter at 1.5-2.0 hrs after reaction initiation. Signal was calculated as follows: Emission 665 nm/Emission 615 nM×10000 for each well.
The compounds of examples 1, 2, 3, 5, 6, 7, 8, 9, 10, 11, 12, 13, 14, 16, 17, 18, 20, 21 were tested and showed an IC₅₀ value of less than 500 nM in this assay. In one embodiment, the present invention relates to a compound which exhibits an IC₅₀ value of less than 500 nM.

### Method of Treating

Another embodiment of the present invention thus relates to a method of using the compounds described above, including salts thereof and corresponding compositions thereof, as cancer chemotherapeutic agents. This method comprises administering to a patient an amount of a compound of this invention, or a pharmaceutically acceptable salt thereof, which is effective to treat the patient's cancer. A patient, for the purpose of this invention, is a mammal, including a human, in need of treatment for a particular cancer. Cancers include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The compounds of this invention can be administered as the sole pharmaceutical agent or in combination with one or more other pharmaceutical agents where the combination causes no unacceptable adverse effects. For example, the compounds of this invention can be combined with known anti-hyper-proliferative, chemotherapeutic, or other indication agents, and the like, as well as with admixtures and combinations thereof.

Optional anti-hyper-proliferative agents which can be added to the composition include but are not limited to compounds listed on the cancer chemotherapy drug regimens in the 11^{th} Edition of the Merck Index, (1996), such as cisplatin.

Other anti-hyper-proliferative agents suitable for use with this invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996) such as idarubicin.

### C. OPERATIVE EXAMPLES RELATING TO PHARMACEUTICAL COMPOSITIONS

The active compound can act systemically, locally or both. For this purpose it can be administered in a suitable manner, such as for example by oral, parenteral, pulmonary, nasal, sublingual, lingual, buccal, rectal, dermal, transdermal, conjunctival or aural administration or in the form of an implant or stent. The active compound can be administered in forms suitable for these modes of administration.

Suitable forms of oral administration are those according to the prior art which function by releasing the active compound rapidly or in a modified or controlled manner and which contain the active compound in a crystalline, amorphous, or dissolved form, for example tablets (which can be uncoated or coated, for example with enteric coatings or coatings which dissolve after a delay in time or insoluble coatings which control the release of the active compound), tablets or films (wafers), which disintegrate rapidly in the oral cavity, films/lyophilisates, capsules (e.g. hard or soft gelatin capsules), dragées, pellets, powders, emulsions, suspensions and solutions. An overview of application forms is given in Remington's Pharmaceutical Sciences, 18^{th} ed. 1990, Mack Publishing Group, Enolo.

Parenteral administration can be carried out by avoiding an absorption step (e.g. by intravenous, intraarterial, intracardial, intraspinal or intralumbar administration) or by including absorption (e.g. by intramuscular, subcutaneous, intracutaneous or intraperitoneal administration). Suitable parenteral administration forms are for example injection and infusion formulations in the form of solutions, suspensions, emulsions, lyophilisates and sterile powders. Such parenteral pharmaceutical compositions are described in Part 8, Chapter 84 of Remington's Pharmaceutical Sciences, 18^{th} ed. 1990, Mack Publishing Group, Enolo.

Suitable forms of administration for the other modes of administration are for example inhalation devices (such as for example powder inhalers, nebulizers), nasal drops, solutions and sprays; tablets or films/wafers for lingual, sublingual or buccal administration or capsules, suppositories, ear and eye preparations, vaginal capsules, aqueous suspensions (lotions or shaking mixtures), lipophilic suspensions, ointments, creams, transdermal therapeutic systems, milky lotions, pastes, foams, dusting powders, implants or stents.

The active compounds can be converted into the abovementioned forms of administration in a manner known to the skilled man and in accordance with the prior art using inert, non-toxic, pharmaceutically suitable auxiliaries. The latter include for example excipients (e.g. microcrystalline cellulose, lactose, mannitol, etc.), solvents (e.g. liquid polyethylene glycols), emulsifiers and dispersants or wetting agents (e.g. sodium dodecyl sulfate, polyoxysorbitan oleate etc.), binders (e.g. polyvinyl pyrrolidone), synthetic and/or natural polymers (e.g. albumin), stabilizers (e.g. antioxidants, such as, for example, ascorbic acid), dyes (e.g. inorganic pigments such as iron oxides) or taste- and/or odour-corrective agents.

The total amount of the active ingredient to be administered will generally range from about 0.01 mg/kg to about 200 mg/kg, and preferably from about 0.1 mg/kg to about 20 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily oral dosage regimen will preferably be from 0.01 to 200 mg/kg of total body weight.

It may however be necessary to deviate from the abovementioned quantities, depending on the body weight, mode of administration, the individual patient response to the active compound, the type of preparation and the time or interval of administration.

If used as active compounds, the compounds according to the invention are preferably isolated in more or less pure form, that is more or less free from residues from the synthetic procedure. The degree of purity can be determined by methods known to the chemist or pharmacist (see Remington's Pharmaceutical Sciences, 18^{th} ed. 1990, Mack Publishing Group, Enolo). Preferably the compounds are greater than 99% pure (w/w), while purities of greater than 95%, 90% or 85% can be employed if necessary.

The compounds according to the invention can be converted into pharmaceutical preparations as follows:

### Tablet:

### Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.
Tablet weight 212 mg, diameter 8 mm, curvature radius 12 mm.

### Preparation:

The mixture of active component, lactose and starch is granulated with a 5% solution (m/m) of the PVP in water. After drying, the granules are mixed with magnesium stearate for 5 min. This mixture is moulded using a customary tablet press (tablet format, see above). The moulding force applied is typically 15 kN.

### Orally Administrable Suspension:

### Composition:

1000 mg of the compound of Example 1, 1000 mg of ethanol (96%), 400 mg of Rhodigel (xanthan gum from FMC, Pennsylvania, USA) and 99 g of water.
A single dose of 100 mg of the compound according to the invention is provided by 10 ml of oral suspension.

### Preparation:

The Rhodigel is suspended in ethanol and the active component is added to the suspension. The water is added with stirring. Stirring is continued for about 6 h until the swelling of the Rhodigel is complete.

It is believed that one skilled in the art, using the preceding information, can utilize the present invention to its fullest extent. It should be apparent to one of ordinary skill in the art that changes and modifications can be made to this invention without departing from the spirit or scope of the invention as it is set forth herein. Other embodiments of the invention will be apparent to the skilled in the art from a consideration of this specification or practice of the invention disclosed herein. It is intended that the specification and examples be considered as exemplary only, with the true scope and spirit of the invention being indicated by the following claims.

We claim:

1. A compound of formula (I) 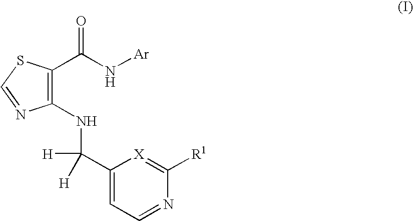
wherein
Ar is selected from the group consisting of 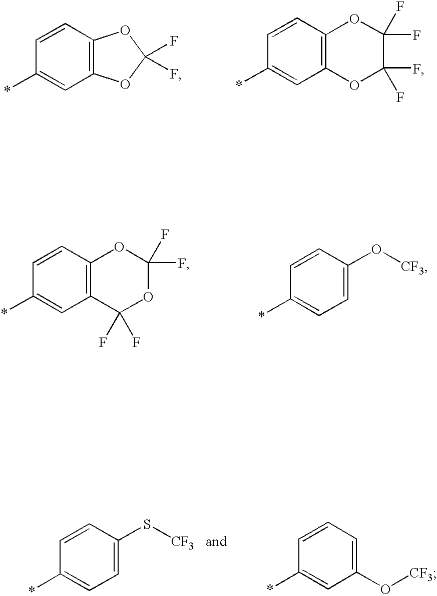
X is CH or N;
R¹ is selected from the group consisting of
H,
halogen, 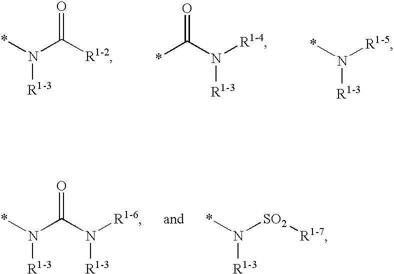
wherein
R¹⁻² is selected from the group consisting of
H,
(C₁-C₄)alkyl,
wherein said (C₁-C₄)alkyl can be substituted with 0, 1, or 2 groups independently selected from hydroxy,
(C₁-C₄)alkylamino,
(C₁-C₄)acyloxy,
(C₁-C₄)alkoxy, and
(C₂-C₄)alkoxy substituted with 0, 1 or 2 (C₁-C₄)alkoxy groups,
5- or 6-membered heteroaryl,
and
phenyl substituted with 0, 1, or 2 groups independently selected from the group consisting of (C₁-C₄)alkyl, halo, nitro, (C₁-C₄)alkoxy and cyano,
and
wherein said (C₁-C₄)alkyl is independently optionally substituted with F up to the perfluoro level;
R¹⁻³ is H or (C₁-C₄)alkyl;
R¹⁻⁴, R¹⁻⁵ and R¹⁻⁶ are selected from the group consisting of
H,
indan-5-yl,
phenyl substituted with 0, 1, or 2 groups independently selected from the group consisting of (C₁-C₄)alkyl, halo, nitro, (C₁-C₄)alkoxy and cyano,
5- or 6-membered heteroaryl substituted with 0, 1 or 2 groups selected from the group consisting of
cyano,
halo,
nitro,
(C₁-C₄)alkyl,
wherein said (C₁-C₄)alkyl is optionally substituted with 0, 1, or 2 groups selected
from
(C₁-C₄)alkylamino,
(C₁-C₄)acyloxy,
(C₁-C₄)alkoxy,
and
(C₂-C₄)alkoxy substituted with up to 0, 1 or 2 (C₁-C₄)alkoxy groups,
(C₃-C₆)cycloalkyl substituted with 0, 1 or 2 groups selected from (C₁-C₄)alkyl, (C₁-C₄)alkoxy, cyano, and halo,
and
(C₁-C₆)alkyl,
wherein said (C₁-C₆)alkyl is independently substituted with 0 or 1 group selected from the group consisting of
NH₂,
(C₁-C₄)alkoxy,
(C₂-C₄)alkoxy independently substituted with 0, 1, 2 or 3 (C₁-C₄)alkoxy and OH groups,
and
independently optionally substituted with fluorine up to the perfluoro level,
carboxyl,
(C₁-C₄)alkoxycarbonyl
(C₁-C₄)alkylamino,
aminocarbonyl,
(C₁-C₄)alkylsulfonyl,
phenyl substituted with 0, 1, or 2 groups independently selected from the group consisting of (C₁-C₄)alkyl, halo, nitro, (C₁-C₄)alkoxy and cyano, 5- or 6-membered heteroaryl independently substituted with 0, 1, 2 or 3 groups selected from the group consisting of (C₁-C₄)alkyl, (C₁-C₄)alkoxy,
cyano, halo, and nitro
and
heterocyclyl independently substituted with 0, 1, 2 or 3 groups selected from the group consisting of (C₁-C₄)alkyl, (C₁-C₄)alkoxy, cyano, and halo,
and
wherein said (C₁-C₆)alkyl is independently substituted with 0, 1 or 2 OH or halo groups,
and
wherein said (C₁-C₆)alkyl is independently optionally substituted with F up to the perfluoro level;
and
R¹⁻³ and R¹⁻⁴, R¹⁻³ and R¹⁻⁵, and R¹⁻³ and R¹⁻⁶, when attached to the same nitrogen atom, may form, together with the N atom to which they are attached, a 5- or 6-membered saturated heterocyclic ring selected from pyrrolidinyl, morpholinyl, thiomorpholinyl and piperizinyl optionally substituted on N with (C₁-C₄)alkyl,
R¹⁻⁷ is independently selected from the group consisting of (C₁-C₄)alkyl,
wherein said (C₁-C₄)alkyl is substituted with 0, 1 or 2 groups selected from the group consisting of
(C₁-C₄)alkylamino,
(C₁-C₄)acyloxy,
(C₁-C₄)alkoxy,
and
(C₂-C₄)alkoxy substituted with 0, 1 or 2
(C₁-C₄)alkoxy groups;
or a pharmaceutically acceptable salt thereof.

2. The compound of claim 1, wherein
Ar is selected from the group consisting of 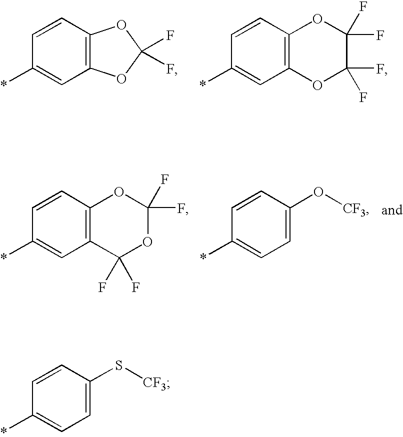
X is CH;
R¹ is selected from the group consisting of 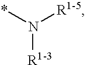
and 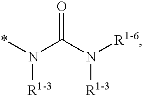
wherein
R¹⁻³ is H or (C₁-C₄)alkyl,
R¹⁻⁵ and R¹⁻⁶ are selected from the group consisting of
H,
indan-5-yl,
phenyl substituted with 0, 1, or 2 groups independently selected from the group consisting of (C₁-C₄)alkyl, halo, nitro, (C₁-C₄)alkoxy and cyano,
5- or 6-membered heteroaryl substituted with 0, 1 or 2 groups selected from the group consisting of
cyano,
halo,
nitro,
(C₁-C₄)alkyl,
wherein said (C₁-C₄)alkyl is optionally substituted with 0, 1, or 2 groups selected from
(C₁-C₄)alkylamino,
(C₁-C₄)acyloxy,
(C₁-C₄)alkoxy,
and
(C₂-C₄)alkoxy substituted with up to 0, 1 or 2 (C₁-C₄)alkoxy groups;
(C₃-C₆)cycloalkyl substituted with 0, 1 or 2 groups selected from the group consisting of (C₁-C₄)alkyl, (C₁-C₄)alkoxy, cyano, and halo;
and
(C₁-C₆)alkyl,
wherein said (C₁-C₆)alkyl is independently substituted with 0 or 1 group selected from the group consisting of
NH₂,
(C₁-C₄)alkoxy,
(C₂-C₄)alkoxy independently substituted with 0, 1, 2 or 3 (C₁-C₄)alkoxy and OH groups,
and
independently optionally substituted with fluorine up to the perfluoro level,
carboxyl,
(C₁-C₄)alkoxycarbonyl
(C₁-C₄)alkylamino,
aminocarbonyl,
(C₁-C₄)alkylsulfonyl,
phenyl substituted with 0, 1, or 2 groups independently selected from the group consisting of (C₁-C₄)alkyl, halo, nitro, (C₁-C₄)alkoxy and cyano,
5- or 6-membered heteroaryl independently substituted with 0, 1, 2 or 3 groups selected from the group consisting of (C₁-C₄)alkyl, (C₁-C₄)alkoxy, cyano, halo, and nitro
and
heterocyclyl is independently substituted with 0, 1, 2 or 3 groups selected from the group consisting of (C₁-C₄)alkyl, (C₁-C₄)alkoxy, cyano, and halo,
and
wherein said (C₁-C₆)alkyl is independently substituted with 0, 1 or 2 OH or halo groups,
and
wherein said (C₁-C₆)alkyl is independently optionally substituted with F up to the perfluoro level;
and
R¹⁻³ and R¹⁻⁵, and R¹⁻³ and R¹⁻⁶, when attached to the same nitrogen atom, may form, together with the N atom to which they are attached, a 5- or 6-membered saturated heterocyclic ring selected from pyrrolidinyl, morpholinyl, thiomorpholinyl and piperizinyl optionally substituted on N with (C₁-C₄)alkyl;
or a pharmaceutically acceptable salt thereof.

3. The compound of claim 1, wherein
Ar is 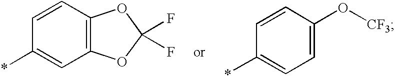
X is CH;
R¹ is selected from 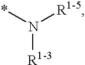
and 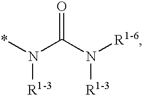
wherein
R¹⁻³ is H,
R¹⁻⁵ is (C₁-C₆)alkyl,
wherein said (C₁-C₆)alkyl is independently substituted with 0 or 1 group selected from
(C₁-C₄)alkoxy,
(C₂-C₄)alkoxy independently substituted with 0, 1, or 2 (C₁-C₄)alkoxy and OH groups,
and
independently optionally substituted with fluorine up to the perfluoro level,
and
wherein said (C₁-C₆)alkyl is independently substituted with 0, 1 or 2 OH or halo groups,
and
wherein said (C₁-C₆)alkyl is independently optionally substituted with F up to the perfluoro level;
R¹⁻⁶ is selected from the group
H,
and
(C₁-C₆)alkyl,
wherein said (C₁-C₆)alkyl is independently substituted with 0 or 1 group selected from
(C₁-C₄)alkoxy,
(C₂-C₄)alkoxy independently substituted with 0, 1, 2 or 3 (C₁-C₄)alkoxy and OH groups,
and
independently optionally substituted with fluorine up to the perfluoro level,
and
wherein said (C₁-C₆)alkyl is independently substituted with 0, 1 or 2 OH or halo groups,
and
wherein said (C₁-C₆)alkyl is independently optionally substituted with F up to the perfluoro level;
or a pharmaceutically acceptable salt thereof.

4. A compound of claim 1 for the treatment or prevention of disorders.

5. A pharmaceutical composition comprising the compound of claim 1.

6. The pharmaceutical composition of claim 5, additionally comprising at least one pharmaceutically acceptable carrier or excipient.

7. A pharmaceutical composition of claim 5 for the treatment or prevention of cancer.

8. A process for preparing the pharmaceutical composition of claim 6, comprising combining at least one compound according to claim 1 with at least one pharmaceutically acceptable carrier or excipient and bringing the resulting combination into a form suitable for said pharmaceutical composition.

9. A use of a compound of claim 1 for manufacturing a pharmaceutical composition for the treatment or prevention of a disease.

10. The use of claim 9, wherein the disease is cancer.

11. A method of treating a disease or condition in a mammal, comprising administering to a mammal in need thereof an effective amount of a compound of claim 1.

12. The method of claim 11, wherein the disease or condition is cancer.
